(19) United States
(12) Patent Application Publication
(10) Pub. No.: US 2026/0217779 A1
(43) Pub. Date: Jul. 30, 2026

(54) CXCR3 LIGAND

(71) Applicant: CHUGAI SEIYAKU KABUSHIKI KAISHA, Tokyo (JP)

(72) Inventors: Yohei YAMAMOTO, Shizuoka (JP); Mika SAKURAI, Shizuoka (JP); Kenta HARAYA, Shizuoka (JP)

(73) Assignee: CHUGAI SEIYAKU KABUSHIKI KAISHA, Tokyo (JP)

(21) Appl. No.: 19/641,701

(22) Filed: Apr. 8, 2026

Related U.S. Application Data
(62) Division of application No. 17/299,629, filed on Dec. 4, 2019, now Pat. No. 12,624,077.

(30) Foreign Application Priority Data
Dec. 4, 2018 (JP) 2018-227353

(51) Int. Cl. C07K 14/52; A61K 38/00; C07K 1/113

(52) U.S. Cl. CPC C07K 14/521; C07K 1/113; A61K 38/00; C07K 2319/30

(57) ABSTRACT

The present disclosure relates to CXCR3 ligands having resistance to DPPIV and having CXCR3-expressing cell migration-inducing activity. The present disclosure specifically relates to N-terminal amino acid modifications and N-terminal amino acid sequences important for resistance to DPPIV and CXCR3-expressing cell migration-inducing activity.

### CROSS-REFERENCE TO RELATED APPLICATIONS

This application is a divisional of U.S. patent application Ser. No. 17/299,629, filed Jun. 3, 2021, which is a U.S. National Phase of PCT Application No. PCT/JP2019/047382, filed Dec. 4, 2019, which claims the benefit of Japanese Patent Application No. 2018-227353, filed Dec. 4, 2018, each of which is incorporated herein by reference in its entirety.

### REFERENCE TO SEQUENCE LISTING SUBMITTED ELECTRONICALLY

The content of the electronically submitted sequence listing (Name: 6663_0400 Sequence_Listing.xml; Size: 504,299 bytes; and Date of Creation: Apr. 6, 2026) filed with the application is incorporated herein by reference in its entirety.

### TECHNICAL FIELD

The present disclosure relates to CXCR3 ligands, methods of producing CXCR3 ligands, use of CXCR3 ligands, and methods of conferring resistance to DPPIV on CXCR3 ligands.

### BACKGROUND ART

Chemokine receptor CXCR3 (also called G Protein-coupled Receptor 9 (GPR9) and CD183) belongs to the CXC chemokine receptor family and is a G protein-coupled receptor that binds to chemokines CXCL9, CXCL10, and CXCL11. CXCR3 is expressed primarily in activated T-helper type 1 (Th1) lymphocytes and cytotoxic T cells, but is also present in natural killer cells, macrophages, dendritic cells, and B lymphocyte subsets. The chemokines CXCL9, CXCL10, and CXCL11 are three naturally-occurring CXCR3 ligands. The interaction of CXCR3 and its ligands is involved in guiding receptor-bearing cells to specific parts of the body, especially sites of inflammation, immune impairment, and immune dysfunction.

CXCL10 (C-X-C motif chemokine 10) is also called “IP10 (interferon gamma-induced protein 10)” or “small inducible cytokine B10” and is a chemokine belonging to the CXC subfamily.

C-X-C motif chemokine 10 (CXCL10) is known to promote T cell migration activity via CXCR3 (NPL 1). Furthermore, it has been reported that direct administration of CXCL10 into a mouse tumor increases T cells in the tumor (NPL 2).

On the other hand, it is known that the two amino acids from N-terminal of CXCL10 are cleaved by Dipeptidyl Peptidase-4 (DPPIV, DPP4) (NPL 1). Cleaved CXCL10 is present even in human blood and is also reported to be present in human tumors, indicating that CXCL10 is cleaved by DPPIV in human blood and tumors (NPLs 3 and 4). Furthermore, since it has been reported that the migration-inducing activity of DPPIV-cleaved CXCL10 is significantly reduced, it is thought that the cleavage could be one of the CXCL10-inactivating mechanisms (NPL 1). The three-dimensional structure of CXCL10 has been elucidated (NPLs 5 and 6), and a predicted structure of a model of a complex with CXCR3 has also been reported (NPL 7). In this complex model, the N-terminus of CXCL10 is arranged in such a way that it penetrates into the inside of CXCR3, and this interaction is thought to be important for CXCR3 activation.

C-X-C motif chemokine 11 (C-X-C motif chemokine ligand 11, CXCL11) is a C-X-C chemokine which is also called I-TAC (Interferon-inducible T-cell alpha chemoattractant) or IP-9 (Interferon-gamma-inducible protein 9), and naturally-occurring CXCL11 is said to bind to CXCR3 more strongly than naturally-occurring CXCL10 and naturally-occurring CXCL9 (NPLs 8 and 9). It is known that the N-terminal sequence of naturally-occurring human CXCL11 is also cleaved by DPPIV (NPL 1).

C-X-C motif chemokine 9 (C-X-C motif chemokine ligand 9, CXCL9) is a C-X-C chemokine which is also called Monokine induced by gamma-interferon (MIG), and the N-terminal sequence of naturally-occurring human CXCL9 is also known to be cleaved by DPPIV (NPL 1).

### CITATION LIST

### Non-Patent Literature

[NPL 1] Proost P, Blood. 2001 Dec. 15; 98 (13): 3554-61.
[NPL 2] Wang P, Cancer Immunol Immunother. 2010 November; 59 (11): 1715-26.
[NPL 3] Decalf J, EMBO Mol Med. 2016 Jun. 1; 8 (6): 679-83.
[NPL 4] Rainczuk A, Int J Cancer. 2014 Feb. 1; 134 (3): 530-41.
[NPL 5] Booth V, Biochemistry. 2002 Aug. 20; 41 (33): 10418-25.
[NPL 6] Swaminathan G J, Structure. 2003 May; 11 (5): 521-32.
[NPL 7] Trotta T, Mol Immunol. 2009 December; 47 (2-3): 332-9.
[NPL 8] Cole K E, The Journal of Experimental Medicine. 187 (12): 2009-21.
[NPL 9] Tensen C P, The Journal of Investigative Dermatology. 112 (5): 716-22.

### SUMMARY OF INVENTION

### Technical Problem

The present disclosure provides CXCR3 ligands having resistance to DPPIV and having CXCR3-expressing cell migration-inducing activity, methods of using these CXCR3 ligands, and methods of producing these CXCR3 ligands. The present disclosure also provides methods of conferring DDPIV resistance on CXCR3 ligands.

### Solution to Problem

The present inventors discovered CXCR3 ligands having resistance to DPPIV and CXCR3-expressing-cell migration-inducing activity, and completed the present disclosure. Specifically, the inventors discovered N-terminal amino acid modifications and N-terminal amino acid sequences that are important for resistance to DPPIV and CXCR3-expressing-cell migration-inducing activity.

The present disclosure specifically includes the embodiments exemplified below:
[A-1] a CXCR3 ligand having resistance to DPPIV and having an activity to cause migration of cells expressing CXCR3;
[A-2] the CXCR3 ligand according to [A-1], wherein the CXCR3 ligand has a C-X-C motif;
[A-3] the CXCR3 ligand according to [A-2], wherein the two cysteines comprised in the C-X-C motif each form a disulfide bond with a cysteine other than that in the C-X-C motif comprised in the CXCR3 ligand;
[A-4] the CXCR3 ligand according to any one of [A-1] to [A-3], wherein the CXCR3 ligand has any of the following sequences (a1) to (a7) at the N-terminus:
   (a1) V-X1-L (X1 is F, G, I, K, L, M, T, V, W, or Y);
   (a2) X2-V-P (X2 is A, F, G, H, I, K, L, M, N, P, Q, R, S, T, V, W, or Y);
   (a3) V-X3-P (X3 is A, F, G, H, I, K, L, M, P, Q, R, S, T, V, W, or Y);
   (a4) P-L-S;
   (a5) X4-F-P (X4 is A, D, E, F, G, H, I, K, L, M, N, P, Q, S, T, V, W, or Y);
   (a6) F-X5-M (X5 is A, D, E, G, H, I, M, N, Q, R, S, T, V, W, or Y); and
   (a7) F-X6-P (X6 is A, D, E, F, G, H, L, M, N, P, Q, S, T, W, or Y);
[A-5] the CXCR3 ligand according to any one of [A-1] to [A-4], wherein the CXCR3 ligand has any of the following sequences (b1) to (b7) at the N-terminus:

(b1)
(SEQ ID NO: 205)
V-X1-L-S-R-T-V-R
(X1 is F, G, I, K, L, M, T, V, W, or Y);
(b2)
(SEQ ID NO: 206)
X2-V-P-L-S-R-T-V-R
(X2 is A, F, G, H, I, K,
L, M, N, P, Q, R, S, T, V, W, or Y);
(b3)
(SEQ ID NO: 207)
V-X3-P-L-S-R-T-V-R;
(X3 is A, F, G, H, I, K, L, M, P, Q, R, S,
T, V, W, or Y)
(b4)
(SEQ ID NO: 208)
P-L-S-R-T-V-R;
(b5)
(SEQ ID NO: 209)
X4-F-P-M-F-K-R-G-R;
(X4 is A, D, E, F, G, H, I,
K, L, M, N, P, Q, S, T, V, W, or Y)
(b6)
(SEQ ID NO: 210)
F-X5-M-F-K-R-G-R;
(X5 is A, D, E, G, H, I,
M, N, Q, R, S, T, V, W, or Y)
and
(b7)
(SEQ ID NO: 211)
F-X6-P-M-F-K-R-G-R
(X6 is A, D, E, F, G, H, L, M, N, P, Q, S,
T, W, or Y);

[A-6] the CXCR3 ligand according to any one of [A-1] to [A-3], wherein the CXCR3 ligand has any of the following sequences (a1) to (a5) at the N-terminus:
   (a1) V-X1-L (X1 is F, G, I, L, M, T, V, W, or Y);
   (a2) X2-V-P (X2 is A, G, I, L, N, Q, S, T, or W);
   (a3) V-X3-P (X3 is A, F, G, I, M, P, T, or V);
   (a4) P-L-S; and
   (a5) X4-F-P (X4 is A, D, E, G, M, N, Q, S, T, V, or Y);
[A-7] the CXCR3 ligand according to any of [A-1] to [A-3] or [A-6], wherein the CXCR3 ligand has any of the following sequences (b1) to (b5) at the N-terminus:

(b1)
(SEQ ID NO: 212)
V-X1-L-S-R-T-V-R
(X1 is F, G, I, L, M, T, V, W, or Y);
(b2)
(SEQ ID NO: 213)
X2-V-P-L-S-R-T-V-R
(X2 is A, G, I, L, N, Q, S, T, or W);
(b3)
(SEQ ID NO: 214)
V-X3-P-L-S-R-T-V-R ;
(X3 is A, F, G, I, M, P, T, or V)
(b4)
(SEQ ID NO: 208)
P-L-S-R-T-V-R;
and
(b5)
(SEQ ID NO: 215)
X4-F-P-M-F-K-R-G-R
(X4 is A, D, E, G, M, N, Q, S, T, V, or Y);

[A-8] the CXCR3 ligand according to any one of [A-2] to [A-7], wherein the C-X-C motif is C-T-C (Cys-Thr-Cys), C-L-C (Cys-Leu-Cys), or C-S-C (Cys-Ser-Cys);
[A-9] the CXCR3 ligand according to any one of [A-2] to [A-8], wherein the CXCR3 ligand further has any of the following (c1) to (c5) at the C-terminus of the C-X-C motif:
   (c1) the sequence from the 12th amino acid to the 77th amino acid of SEQ ID NO: 60;
   (c2) the sequence from the 12th amino acid to the 73rd amino acid of SEQ ID NO: 61;
   (c3) the sequence from the 12th amino acid to the 103rd amino acid of SEQ ID NO: 62;
   (c4) the sequence from the 12th amino acid to the 77th amino acid of SEQ ID NO: 1; and
   (c5) the sequence from the 12^{th} amino acid to the 77^{th} amino acid of SEQ ID NO: 63;
[A-10] the CXCR3 ligand according to any one of [A-1] to [A-9], wherein the sequence of the CXCR3 ligand is any of the following (d1) to (d7):
   (d1) a sequence shown by any one of SEQ ID NOs: 2 to 57, 92 to 147, and 149 to 204;
   (d2) a sequence showing 90% or more sequence identity to SEQ ID NO: 60;
   (d3) a sequence showing 90% or more sequence identity to SEQ ID NO: 61;
   (d4) a sequence showing 90% or more sequence identity to SEQ ID NO: 62;
   (d5) a sequence showing 90% or more sequence identity to SEQ ID NO: 63;
   (d6) a sequence showing 90% or more sequence identity to SEQ ID NO: 1; and
   (d7) a sequence comprising 10 or less amino acid substitutions, insertions, or deletions to a sequence selected from SEQ ID NOs: 1 to 57, 60 to 63, 92 to 147, and 149 to 204;
[A-11] the CXCR3 ligand according to any one of [A-1] to [A-10], wherein resistance to DPPIV indicates that, after treatment at 37° C. for 1 hour in the presence of 0.2 mg/ml of the CXCR3 ligand or CXCR3 ligand-human Fc fusion protein and 200 nM human DPPIV, the two N-terminal residues of the CXCR3 ligand are not lost;
[A-12] the CXCR3 ligand according to any one of [A-1] to [A-11], wherein the activity of the CXCR3 ligand to cause migration of cells expressing CXCR3 is 30% or more of the activity of a naturally-occurring human CXCL10 to cause migration of cells expressing CXCR3, or 25% or more of the activity of a naturally-occurring human CXCL11 to cause migration of cells expressing CXCR3;
[A-13] the CXCR3 ligand according to any one of [A-1] to [A-12], wherein the CXCR3 ligand concentration when the activity of the CXCR3 ligand to cause migration of cells expressing CXCR3 is at maximum is lower than the concentration of a naturally-occurring human CXCL10 and/or naturally-occurring human CXCL11 when the activity of the naturally-occurring human CXCL10 and/or the naturally-occurring human CXCL11 to cause migration of cells expressing CXCR3 is at maximum; and
[A-14] the CXCR3 ligand according to any one of [A-1] to [A-13], wherein the cells expressing CXCR3 are cells isolated from a transfectant or a living body expressing CXCR3.

The present disclosure also includes the embodiments exemplified below:
[B-1] a CXCR3 ligand having any of the following sequences (a1) to (a7) at the N-terminus:
   (a1) V-X1-L (X1 is F, G, I, K, L, M, T, V, W, or Y);
   (a2) X2-V-P (X2 is A, F, G, H, I, K, L, M, N, P, Q, R, S, T, V, W, or Y);
   (a3) V-X3-P (X3 is A, F, G, H, I, K, L, M, P, Q, R, S, T, V, W, or Y);
   (a4) P-L-S;
   (a5) X4-F-P (X4 is A, D, E, F, G, H, I, K, L, M, N, P, Q, S, T, V, W, or Y);
   (a6) F-X5-M (X5 is A, D, E, G, H, I, M, N, Q, R, S, T, V, W, or Y); and
   (a7) F-X6-P (X6 is A, D, E, F, G, H, L, M, N, P, Q, S, T, W, or Y);
[B-2] the CXCR3 ligand according to [B-1], which has any of the following sequences (b1) to (b7) at the N-terminus:

(b1)
(SEQ ID NO: 205)
V-X1-L-S-R-T-V-R
(X1 is F, G, I, K, L, M, T, V, W, or Y);
(b2)
(SEQ ID NO: 206)
X2-V-P-L-S-R-T-V-R;
(X2 is A, F, G, H, I, K, L, M, N, P, Q, R,
S, T, V, W, or Y)
(b3)
(SEQ ID NO: 207)
V-X3-P-L-S-R-T-V-R;
(X3 is A, F, G, H, I, K, L, M, P, Q, R, S,
T, V, W, or Y)
(b4)
(SEQ ID NO: 208)
P-L-S-R-T-V-R;
(b5)
(SEQ ID NO: 209)
X4-F-P-M-F-K-R-G-R;
(X4 is A, D, E, F, G, H, I, K, L, M, N, P,
Q, S, T, V, W, or Y)
(b6)
(SEQ ID NO: 210)
F-X5-M-F-K-R-G-R;
(X5 is A, D, E, G, H, I, M, N, Q, R, S,
T, V, W, or Y)
and
(b7)
(SEQ ID NO: 211)
F-X6-P-M-F-K-R-G-R;
(X6 is A, D, E, F, G, H, L, M, N, P, Q,
S, T, W, or Y)

[B-3] the CXCR3 ligand according to [B-1] or [B-2], which has a C-X-C motif on the C-terminal side of the sequences (a1) to (a7);
[B-4] the CXCR3 ligand according to [B-2], which has a C-X-C motif at the C-terminus of the sequences (b1) to (b7);
[B-5] a CXCR3 ligand having any of the following sequences (a1) to (a5) at the N-terminus:
   (a1) V-X1-L (X1 is F, G, I, L, M, T, V, W, or Y);
   (a2) X2-V-P (X2 is A, G, I, L, N, Q, S, T, or W);
   (a3) V-X3-P (X3 is A, F, G, I, M, P, T, or V);
   (a4) P-L-S; and
   (a5) X4-F-P (X4 is A, D, E, G, M, N, Q, S, T, V, or Y);
[B-6] the CXCR3 ligand according to [B-5], which has any of the following sequences (b1) to (b5) at the N-terminus:

(b1)
(SEQ ID NO: 212)
V-X1-L-S-R-T-V-R;
(X1 is F, G, I, L, M, T, V, W, or Y)
(b2)
(SEQ ID NO: 213)
X2-V-P-L-S-R-T-V-R;
(X2 is A, G, I, L, N, Q, S, T, or W)
(b3)
(SEQ ID NO: 214)
V-X3-P-L-S-R-T-V-R;
(X3 is A, F, G, I, M, P, T, or V)
(b4)
(SEQ ID NO: 208)
P-L-S-R-T-V-R;
and
(b5)
(SEQ ID NO: 215)
X4-F-P-M-F-K-R-G-R;
(X4 is A, D, E, G, M, N, Q, S, T, V, or Y)

[B-7] the CXCR3 ligand according to [B-5] or [B-6], which has a C-X-C motif on the C-terminal side of the sequences (a1) to (a5);
[B-8] the CXCR3 ligand according to [B-6], which has a C-X-C motif at the C-terminus of the sequences (b1) to (b5);
[B-9] the CXCR3 ligand according to any one of [B-3], [B-4], [B-7], and [B-8], wherein the C-X-C motif is C-T-C (Cys-Thr-Cys), C-L-C (Cys-Leu-Cys), or C-S-C (Cys-Ser-Cys);
[B-10] the CXCR3 ligand according to any one of [B-3], [B-4], [B-7], [B-8], and [B-9], wherein the CXCR3 ligand further has any of the following (c1) to (c5) at the C-terminus of the C-X-C motif:
   (c1) the sequence from the 12th amino acid to the 77th amino acid of SEQ ID NO: 60;
   (c2) the sequence from the 12th amino acid to the 73rd amino acid of SEQ ID NO: 61;
   (c3) the sequence from the 12th amino acid to the 103rd amino acid of SEQ ID NO: 62;
   (c4) the sequence from the 12th amino acid to the 77th amino acid of SEQ ID NO: 1; and
   (c5) the sequence from the 12^{th} amino acid to the 77^{th} amino acid of SEQ ID NO: 63; and
[B-11] the CXCR3 ligand according to any one of [B-1] to [B-10], wherein the sequence of the CXCR3 ligand is any of the following (d1) to (d7):
   (d1) a sequence shown by any one of SEQ ID NOs: 2 to 57, 92 to 147, and 149 to 204;
   (d2) a sequence showing 90% or more sequence identity to SEQ ID NO: 60;
   (d3) a sequence showing 90% or more sequence identity to SEQ ID NO: 61;
   (d4) a sequence showing 90% or more sequence identity to SEQ ID NO: 62;
   (d5) a sequence showing 90% or more sequence identity to SEQ ID NO: 63;
   (d6) a sequence showing 90% or more sequence identity to SEQ ID NO: 1; and
   (d7) a sequence comprising 10 or less amino acid substitutions, insertions, or deletions to a sequence selected from SEQ ID NOs: 1 to 57, 60 to 63, 92 to 147, and 149 to 204.

The present disclosure also encompasses the embodiments exemplified below:
[C-1] an isolated nucleic acid encoding the CXCR3 ligand according to any one of [A-1] to [B-11];
[C-2] a host cell comprising the nucleic acid according to [C-1];
[C-3] a method of producing the CXCR3 ligand according to any one of [A-1] to [B-11], wherein the method comprises culturing the host cell according to [C-2] such that the CXCR3 ligand is produced;
[C-4] the method according to [C-3], further comprising recovering the CXCR3 ligand from the host cell;
[C-5] a fusion protein comprising the CXCR3 ligand according to any one of [A-1] to [B-11]
[C-6] the fusion protein according to [C-5], wherein the CXCR3 ligand according to any one of [A-1] to [B-11] and an antibody Fc region are fused;
[C-7] the fusion protein according to [C-5], wherein the CXCR3 ligand according to any one of [A-1] to [B-11] and an intact antibody or an antibody fragment are fused;
[C-8] the fusion protein according to [C-6] or [C-7], wherein the CXCR3 ligand and the antibody Fc region, or the CXCR3 ligand and the intact antibody or antibody fragment are fused via a linker; and
[C-9] a pharmaceutical composition comprising the CXCR3 ligand according to any one of [A-1] to [B-11] or the fusion protein according to any one of [C-5] to [C-8].

The present disclosure also encompasses the embodiments exemplified below:
[D-1] a method of conferring resistance to DPPIV on a parent CXCR3 ligand in which the 2^{nd} amino acid from the N-terminus is P or A, wherein the method comprises any of the following:
   (1) substituting the 2^{nd} amino acid from the N-terminus of the parent CXCR3 ligand from P or A to F, G, I, K, L, M, T, V, W, or Y;
   (2) further adding A, F, G, H, I, K, L, M, N, P, Q, R, S, T, V, W, or Y to the N-terminus of the parent CXCR3 ligand;
   (3) inserting A, F, G, H, I, K, L, M, P, Q, R, S, T, V, W, or Y between the 1^{st} and 2^{nd} amino acids at the N-terminus of the parent CXCR3 ligand; and
   (4) deleting V at the N-terminus of the parent CXCR3 ligand;
[D-2] a method of conferring resistance to DPPIV on a parent CXCR3 ligand in which the 2^{nd} amino acid from the N-terminus is P or A, wherein the method comprises any of the following:
   (1) substituting the 2^{nd} amino acid from the N-terminus of the parent CXCR3 ligand from P or A to F, G, I, L, M, T, V, W, or Y;
   (2) further adding A, G, I, L, N, Q, S, T, or W to the N-terminus of the parent CXCR3 ligand;
   (3) inserting A, F, G, I, M, P, T, or V between the 1^{st} and 2^{nd} second amino acids at the N-terminus of the parent CXCR3 ligand; and
   (4) deleting V at the N-terminus of the parent CXCR3 ligand;
[D-3] the method according to [D-1] or [D-2], wherein the parent CXCR3 ligand further has a C-X-C motif;
[D-4] the method according to [D-3], wherein the C-X-C motif is C-T-C (Cys-Thr-Cys), C-L-C (Cys-Leu-Cys), or C-S-C (Cys-Ser-Cys);
[D-5] the method according to any one of [D-1] to [D-4], wherein the parent CXCR3 ligand is either a naturally-occurring human CXCL10 or a variant thereof, a naturally-occurring human CXCL11 or a variant thereof, or a naturally-occurring human CXCL9 or a variant thereof;
[D-6] the method according to [D-1] to [D-5], wherein the 1^{st} amino acid at the N-terminus of the parent CXCR3 ligand is V;
[D-7] the method according to any one of [D-1] to [D-6], wherein the N-terminal sequence of the parent CXCR3 ligand is V-P-L-S-R-T-V-R (SEQ ID NO: 86) or V-A-L-S-R-T-V-R (SEQ ID NO: 87); and
[D-8] the method according to [D-7], wherein the C-X-C motif is located at the C-terminus of the N-terminal sequence “V-P-L-S-R-T-V-R (SEQ ID NO: 86)” or “V-A-L-S-R-T-V-R (SEQ ID NO: 87)”.

The present disclosure also encompasses the embodiments exemplified below:
[E-1] a method of producing a CXCR3 ligand having resistance to DPPIV, wherein the method carries out any of the following modifications to a parent CXCR3 ligand in which the 2^{nd} amino acid from the N-terminus is P or A:
   (1) substituting the 2^{nd} amino acid from the N-terminus of the parent CXCR3 ligand from P or A to F, G, I, K, L, M, T, V, W, or Y;
   (2) further adding A, F, G, H, I, K, L, M, N, P, Q, R, S, T, V, W, or Y to the N-terminus of the parent CXCR3 ligand;
   (3) inserting A, F, G, H, I, K, L, M, P, Q, R, S, T, V, W, or Y between the 1^{st} and 2^{nd} amino acids at the N-terminus of the parent CXCR3 ligand; and
   (4) deleting the V at the N-terminus of the parent CXCR3 ligand;
[E-2] a method of producing a CXCR3 ligand having resistance to DPPIV, wherein the method carries out any of the following modifications to a parent CXCR3 ligand in which the 2^{nd} amino acid from the N-terminus is P or A:
   (1) substituting the 2^{nd} amino acid from the N-terminus of the parent CXCR3 ligand from P or A to F, G, I, L, M, T, V, W, or Y;
   (2) further adding A, G, I, L, N, Q, S, T, or W to the N-terminus of the parent CXCR3 ligand;
   (3) inserting A, F, G, I, M, P, T, or V between the 1^{st} and 2^{nd} second amino acids at the N-terminus of the parent CXCR3 ligand; and
   (4) deleting V at the N-terminus of the parent CXCR3 ligand;
[E-3] the method according to [E-1] or [E-2], wherein the parent CXCR3 ligand further has a C-X-C motif;
[E-4] the method according to [E-3], wherein the C-X-C motif is C-T-C (Cys-Thr-Cys), C-L-C (Cys-Leu-Cys), or C-S-C (Cys-Ser-Cys);
[E-5] the method according to any one of [E-1] to [E-4], wherein the parent CXCR3 ligand is either a naturally-occurring human CXCL10 or a variant thereof, a naturally-occurring human CXCL11 or a variant thereof, or a naturally-occurring human CXCL9 or a variant thereof;
[E-6] the method according to [E-1] to [E-5], wherein the 1^{st} amino acid at the N-terminus of the parent CXCR3 ligand is V;
[E-7] the method according to any one of [E-1] to [E-6], wherein the N-terminal sequence of the parent CXCR3 ligand is V-P-L-S-R-T-V-R (SEQ ID NO: 86) or V-A-L-S-R-T-V-R (SEQ ID NO: 87);
[E-8] the method according to [E-7], wherein the parent C-X-C motif is located at the C-terminus of the N-terminal sequence “V-P-L-S-R-T-V-R (SEQ ID NO: 86)” or “V-A-L-S-R-T-V-R (SEQ ID NO: 87)”; and
[E-9] a CXCR3 ligand produced by a method of any one of [E-1] to [E-8].

The present disclosure also encompasses the embodiments exemplified below:
[F-1] a method of conferring resistance to DPPIV on a parent CXCR3 ligand in which the 2^{nd} amino acid from the N-terminus is P, wherein the method comprises any of the following:
   (1) further adding A, D, E, F, G, H, I, K, L, M, N, P, Q, S, T, V, W, or Y to the N-terminus of the parent CXCR3 ligand;
   (2) substituting the 2^{nd} amino acid from the N-terminus of the parent CXCR3 ligand from P to A, D, E, G, H, I, M, N, Q, R, S, T, V, W, or Y; and
   (3) inserting A, D, E, F, G, H, L, M, N, P, Q, S, T, W, or Y between the 1^{st} and 2^{nd} amino acids at the N-terminus of the parent CXCR3 ligand;
[F-2] a method of conferring resistance to DPPIV on a parent CXCR3 ligand in which the 2^{nd} amino acid from the N-terminus is P, wherein the method comprises further adding A, D, E, G, M, N, Q, S, T, V, or Y to the N-terminus of the parent CXCR3 ligand;
[F-3] the method according to [F-1] or [F-2], wherein the parent CXCR3 ligand further has a C-X-C motif;
[F-4] the method according to [F-3], wherein the C-X-C motif is C-T-C (Cys-Thr-Cys), C-L-C (Cys-Leu-Cys), or C-S-C (Cys-Ser-Cys);
[F-5] the method according to any one of [F-1] to [F-4], wherein the parent CXCR3 ligand is either a naturally-occurring human CXCL10 or a variant thereof, a naturally-occurring human CXCL11 or a variant thereof, or a naturally-occurring human CXCL9 or a variant thereof;
[F-6] the method according to [F-1] to [F-5], wherein the 1^{st} amino acid at the N-terminus of the parent CXCR3 ligand is F;
[F-7] the method according to any one of [F-1] to [F-6], wherein the N-terminal sequence of the parent CXCR3 ligand is F-P-M-F-K-R-G-R (SEQ ID NO: 91); and
[F-8] the method according to [F-7], wherein the C-X-C motif is located at the C-terminus of the N-terminal sequence “F-P-M-F-K-R-G-R (SEQ ID NO: 91)”.

The present disclosure also encompasses the embodiments exemplified below:
[G-1] a method of producing a CXCR3 ligand having resistance to DPPIV, wherein the method carries out any of the following modifications to a parent CXCR3 ligand in which the 2^{nd} amino acid from the N-terminus is P:
   (1) further adding A, D, E, F, G, H, I, K, L, M, N, P, Q, S, T, V, W, or Y to the N-terminus of the parent CXCR3 ligand;
   (2) substituting the 2^{nd} amino acid from the N-terminus of the parent CXCR3 ligand from P to A, D, E, G, H, I, M, N, Q, R, S, T, V, W, or Y; and
   (3) inserting A, D, E, F, G, H, L, M, N, P, Q, S, T, W, or Y between the 1^{st} and 2^{nd} amino acids at the N-terminus of the parent CXCR3 ligand;
[G-2] a method of producing a CXCR3 ligand having resistance to DPPIV, wherein the method carries out, to a parent CXCR3 ligand in which the 2^{nd} amino acid from the N-terminus is P, the modification of further adding A, D, E, G, M, N, Q, S, T, V, or Y to the N-terminus of the parent CXCR3 ligand;
[G-3] the method according to [G-1] or [G-2], wherein the parent CXCR3 ligand further has a C-X-C motif;
[G-4] the method according to [G-3], wherein the C-X-C motif is C-T-C (Cys-Thr-Cys), C-L-C (Cys-Leu-Cys), or C-S-C (Cys-Ser-Cys);
[G-5] the method according to any one of [G-1] to [G-4], wherein the parent CXCR3 ligand is either a naturally-occurring human CXCL10 or a variant thereof, a naturally-occurring human CXCL11 or a variant thereof, or a naturally-occurring human CXCL9 or a variant thereof;
[G-6] the method according to [G-1] to [G-5], wherein the 1^{st} amino acid at the N-terminus of the parent CXCR3 ligand is F;
[G-7] the method according to any one of [G-1] to [G-6], wherein the N-terminal sequence of the parent CXCR3 ligand is F-P-M-F-K-R-G-R (SEQ ID NO: 91);
[G-8] the method according to [G-7], wherein the parent C-X-C motif is located at the C-terminus of the N-terminal sequence “F-P-M-F-K-R-G-R (SEQ ID NO: 91)”; and
[G-9] a CXCR3 ligand produced by a method of any one of [G-1] to [G-8].

### BRIEF DESCRIPTION OF DRAWINGS

FIG. 1A shows a schematic diagram of an hCXCL10 variant Fc fusion.

FIG. 1B shows the positions of the amino acid modifications in and around the DPPIV recognition/cleavage site on the sequence FLTLSGIQGVPLSRTVRC (SEQ ID NO: 216) for producing hCXCL10 variants.

FIG. 2 shows the cell migration-inducing activity of hCXCL10 (PeproTech, cat 300-12) and hCXCL10R75A.

FIG. 3a shows the cell migration-inducing activity of each hCXCL10 variant Fc fusion.

FIG. 3b shows the cell migration-inducing activity of each hCXCL10 variant Fc fusion.

FIG. 4A shows a schematic diagram of an hCXCL11 or hITIP variant Fc fusion.

FIG. 4B shows the positions of the amino acid modifications in and around the DPPIV recognition/cleavage site on the sequence VATATGVHSFPMFKRGRC (SEQ ID NO: 217) for producing hCXCL11 or hITIP variants.

FIG. 5 shows a comparison of cell migration-inducing activities of hCXCL10 variants before and after DPPIV treatment.

FIG. 6-1 shows the cell migration-inducing activity of each hCXCL11 variant Fc fusion.

FIG. 6-2 shows a continuation of FIG. 6-1.

FIG. 6-3 shows a continuation of FIG. 6-2. The activities shown in FIG. 6-1 to FIG. 6-3 are from the same experiment round.

FIG. 7a shows the cell migration-inducing activity of each hITIP variant Fc fusion.

FIG. 7b shows the cell migration-inducing activity of each hITIP variant Fc fusion.

FIG. 7c shows the cell migration-inducing activity of each hITIP variant Fc fusion.

### DESCRIPTION OF EMBODIMENTS

The following definitions and detailed descriptions are provided to facilitate the understanding of the present disclosure explained herein.

### Amino Acids

In the present specification, each amino acid is indicated by one-letter code or three-letter code, or both, as represented by, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Amino Acid Modifications

For the modification of an amino acid in the amino acid sequence of a CXCR3 ligand, a method known in the art such as site-directed mutagenesis (Kunkel et al. (Proc. Natl. Acad. Sci. USA (1985) 82, 488-492)) or overlap extension PCR can be appropriately used. Several methods known in the art can also be used as amino acid modification methods for substituting an amino acid with an amino acid other than a naturally-occurring amino acid (Annu. Rev. Biophys. Biomol. Struct. (2006) 35, 225-249; and Proc. Natl. Acad. Sci. U.S.A. (2003) 100 (11), 6353-6357). For example, a tRNA-containing cell-free translation system (Clover Direct (Protein Express)) in which a non-naturally-occurring amino acid is bound to an amber suppressor tRNA complementary to the UAG codon (amber codon), which is a stop codon, is also preferably used.

In the present specification, the term “and/or” used to represent amino acid modification sites is meant to include every combination in which “and” and “or” are appropriately combined. Specifically, for example, the phrase “amino acids at positions 33, 55, and/or 96 are substituted” includes the following variations of amino acid modification: (a) position 33, (b) position 55, (c) position 96, (d) positions 33 and 55, (e) positions 33 and 96, (f) positions 55 and 96, and (g) positions 33, 55, and 96. Alternatively, the phrase “amino acids at positions 33, 55, and/or 96 are substituted” is synonymous to the phrase “an amino acid at least one (one, two, or three) position selected from the group consisting of positions 33, 55, and 96 is substituted”.

In the present specification, an expression in which the one-letter codes or three-letter-codes of amino acids before and after modification are written prior to and subsequent to a number representing a particular position can be appropriately used for representing an amino acid substitution. For example, the modification P2A or Pro2Ala used for substituting an amino acid contained in a CXCR3 ligand represents the substitution of Pro at position 2 from the N-terminus of the CXCR3 ligand with Ala. Specifically, the number represents an amino acid position as counted from the N-terminus of the CXCR3 ligand; the one-letter code or three-letter code of the amino acid written prior to the number represents the amino acid before the substitution; and the one-letter code or three-letter code of the amino acid subsequent to the number represents the amino acid after the substitution.

In the present specification, an expression showing “ins” and a one-letter code or three-letter-code of an inserted amino acid subsequent to a particular position of insertion can be appropriately used for representing an amino acid insertion. In order to express the position of insertion, an expression showing the one-letter code or three-letter code of the amino acid before and after the position of insertion and the position can be appropriately used. For example, the modification V1_P2insA or Val1_Pro2insAla, which is used when inserting an amino acid into the amino acid sequence contained in a CXCR3 ligand, represents the insertion of Ala between the amino acid Val at position 1 and the amino acid Pro at position 2 from the N-terminus of the CXCR3 ligand. When inserting an amino acid to the N-terminal side of the amino acid Val at position 1 from the N-terminus of the CXCR3 ligand, for example, the expression G-1_V1insA or Gly-1_Val1insAla is used to show the insertion of Ala between Val at position 1 from the N-terminus of the CXCR3 ligand and Gly in the signal sequence positioned next to said Val at the N-terminal side, and since Gly in the signal sequence is the amino acid at position 1 further at the N-terminal side than the N-terminus of the CXCR3 ligand, the position is expressed as “−1”.

### CXCR3

Chemokine receptor CXCR3 (also called G Protein-coupled Receptor 9 (GPR9) and CD183) belongs to the CXC chemokine family and is a G Protein-coupled receptor that binds to the chemokines CXCL9, CXCL10, and CXCL11. CXCR3 is expressed primarily in activated T-helper type 1 (Th1) lymphocytes, but is also present in natural killer cells, macrophages, dendritic cells, and B lymphocyte subsets. The chemokines CXCL9, CXCL10, and CXCL11 are three naturally-occurring CXCR3 ligands. The interaction of CXCR3 and its ligands (hereinafter referred to as the CXCR3 axis) is involved in guiding receptor-bearing cells to specific parts of the body, especially sites of inflammation, immune impairment, and immune dysfunction. Unless otherwise indicated, the term “CXCR3” as used herein indicates any natural CXCR3 from any vertebrate source, including mammals such as primates (e.g., humans) and rodents (e.g., mice and rats). The entire amino acid sequence of human CXCR3 is shown by Refseq: NP_001495 (SEQ ID NO: 88).

### Chemokines and CXC Chemokines

Chemokines are a family of homogeneous serum proteins of 7 to 16 kDa originally characterized by their ability to induce leukocyte migration. Most of chemokines have four characteristic cysteines (Cys) and are classified into C-X-C (or alpha, CXC), C-C (or beta), C (or gamma), and CX3C (or delta) chemokine classes, according to motifs displayed by the first two cysteines. Two disulfide bonds are formed between the first and third cysteines and between the second and fourth cysteines. In general, the disulfide bridges are considered necessary. Clark-Lewis and collaborators have reported that the disulfide bonds are crucial for the chemokine activity of at least CXCL10 (Clark-Lewis et al., J. Biol. Chem. 269:16075-16081, 1994).

Subfamilies of C-X-C (or alpha, CXC) are further classified, according to the presence of an ELR motif (Glu-Leu-Arg) preceding the first cysteine, into two groups: ELR-CXC chemokines and non-ELR-CXC chemokines (see e.g., Clark-Lewis, supra; and Belperio et al., J. Leukoc. Biol. 68:1-8, 2000). CXCL10, CXCL11, and CXCL9 are all non-ELR-CXC chemokines.

### CXCL10

CXC motif chemokine 10 (C-X-C motif chemokine ligand 10, CXCL10) is a C-X-C chemokine, which is also called interferon-induced protein-10 (IP-10). It is induced by interferon-γ and TNF-α and produced by keratinocytes, endothelial cells, fibroblasts, and monocytes. CXCL10 is thought to play a role in the recruitment of activated T cells to sites of tissue inflammation (Dufour, et al., J Immunol., 168:3195-204, 2002). In addition, CXCL10 may play a role in hypersensitivity reactions. It may also play a role in the development of inflammatory demyelinating neuropathy (Kieseier, et al., Brain 125:823-34, 2002).

Researches indicate that CXCL10 may be useful in the engraftment of stem cells following transplantation (Nagasawa, T., Int. J. Hematol. 72:408-11, 2000), in the mobilization of stem cells (Gazitt, Y., J. Hematother Stem Cell Res 10:229-36, 2001; and Hattori et al., Blood 97:3354-59, 2001), and in an enhancement of antitumor immunity (Nomura et al., Int. J. Cancer 91:597-606, 2001; and Mach and Dranoff, Curr. Opin. Immunol. 12:571-75, 2000). For example, previous reports known to those skilled in the art discuss the biological activity of chemokine (Bruce, L. et al., Methods in Molecular Biology (2000) vol. 138, pp. 129-134; Raphaele, B. et al., Methods in Molecular Biology (2000) vol. 138, pp. 143-148; and Paul D. Ponath et al., Methods in Molecular Biology (2000) vol. 138, pp. 113-120). The physiological activity of CXCL10 is exerted by binding to the chemokine receptor CXCR3 expressed on the cell surface (Booth V. et al, Biochemistry. 41 (33): 10418-25).

Unless otherwise indicated, the term “CXCL10” as used herein refers to any natural CXCL10 from any vertebrate source, including mammals such as primates (e.g., humans) and rodents (e.g., mice and rats). The term refers not to the “full-length”, unprocessed CXCL10, but to mature CXCL10 secreted extracellularly as a result of intracellular processing. As used herein, those that have not undergone processing are referred to as CXCL10 precursors. The term also encompasses naturally-occurring mutants of CXCL10 such as splice mutants and allelic mutants. Naturally-occurring human CXCL10 is expressed as a CXCL10 precursor (Refseq Accession number: NP_001556) and then secreted extracellularly as a protein with the sequence set forth in SEQ ID NO: 60. The complete amino acid sequence of the rhesus monkey CXCL10 precursor is indicated by Refseq Accession number: AKK95955, and the complete amino acid sequence of the mouse CXCL10 precursor is indicated by Refseq Accession number: NP_067249.

Natural CXCL10 or naturally-occurring CXCL10 mutants to which artificial amino acid modifications have been added are called “CXCL10 variants”.

### CXCL11

C-X-C motif chemokine 11 (CXC motif chemokine ligand 11, CXCL11) is a C-X-C chemokine, which is also called I-TAC (Interferon-inducible T-cell alpha chemoattractant) or IP-9 (Interferon-gamma-inducible Protein 9). CXCL11 gene expression is strongly induced by IFN-γ and IFN-β, and also by IFN-α (Rani M R, The Journal of Biological Chemistry. 271 (37): 22878-84).

CXCL11 has a biological activity to activate T cells. CXCL11 is known to exert its biological activity by binding to the chemokine receptor CXCR3 expressed on the cell surface, and naturally-occurring CXCL11 is said to bind to CXCR3 more strongly than naturally-occurring CXCL10 and naturally-occurring CXCL9 (Cole K E., The Journal of Experimental Medicine. 187 (12): 2009-21; Tensen C P, The Journal of Investigative Dermatology. 112 (5): 716-22).

The term “CXCL11” as used herein refers to any natural CXCL11 from any vertebrate source, including mammals such as primates (e.g., humans) and rodents (e.g., mice and rats), unless otherwise indicated. The term refers not to a “full-length”, unprocessed CXCL11, but to a mature CXCL11 that is secreted extracellularly as a result of intracellular processing. As used herein, those that have not undergone processing are referred to as CXCL11 precursors. The term also includes naturally-occurring mutants of CXCL11, such as splice mutants and allelic mutants. Natural human CXCL11 is expressed as a CXCL11 precursor (Refseq Accession number: NP_005400) and then secreted extracellularly as a protein with the sequence set forth in SEQ ID NO: 61.

Natural CXCL11 or naturally-occurring CXCL11 mutants to which artificial amino acid modifications have been added are called “CXCL11 variants”.

### CXCL9

C-X-C motif chemokine 9 (C-X-C motif chemokine ligand 9, CXCL9) is a C-X-C chemokine, which is also called Monokine induced by gamma interferon (MIG). CXCL9 is an IFN-γ-induced T-cell chemoattractant and is known to exert its biological activity by binding to the chemokine receptor CXCR3 expressed on the cell surface.

The term “CXCL9” as used herein refers to any natural CXCL9 from any vertebrate source, including mammals such as primates (e.g., humans) and rodents (e.g., mice and rats), unless otherwise indicated. The term refers not to “full-length”, unprocessed CXCL9, but to mature CXCL9 that is secreted extracellularly as a result of intracellular processing. As used herein, those that have not undergone processing are referred to as CXCL9 precursors. The term also includes naturally-occurring mutants of CXCL9, such as splice mutants and allelic mutants. Natural human CXCL9 is expressed as a CXCL9 precursor (Refseq Accession number: NP_002407) and then secreted extracellularly as a protein with the sequence set forth in SEQ ID NO: 62.

Natural CXCL9 or naturally-occurring CXCL9 mutants to which artificial amino acid modifications have been added are called “CXCL9 variants”.

Chimeric proteins can be prepared by fusing parts of CXCL10, CXCL11, CXCL9 and such with each other. For example, a human CXCL10-human CXCL11 chimeric protein (hITIP) (SEQ ID NO: 63) in which the 1^{st} to 24^{th} amino acid residues of human CXCL11 (SEQ ID NO: 61) and the 25^{th} to 77^{th} amino acid residues of a human CXCL10 variant (SEQ ID NO: 1) are bound can be prepared. Such chimeric proteins can also be called CXCR3 ligands as long as they can bind to CXCR3. Furthermore, even in such chimeric proteins, the two N-terminal amino acids may be cleaved by DPPIV. The human CXCL10-human CXCL11 chimeric protein (hITIP) (SEQ ID NO: 63), in which the 1^{st} to 24^{th} amino acid residues of human CXCL11 (SEQ ID NO: 61) and the 25^{th} to 77^{th} amino acid residues of a human CXCL10 variant (SEQ ID NO: 1) are bound, and to which further amino acid modifications have been added is called an “hITIP variant”.

### Method for Measuring the Binding Between a CXCR3 Ligand and CXCR3

The binding activity of a CXCR3 ligand to CXCR3 can be assessed by a well-known method such as FACS, an ELISA format, a BIACORE method using amplified luminescent proximity homogeneous assay (ALPHA) screening or surface plasmon resonance (SPR) phenomena, or bio-layer interferometry (BLI) (Octet) (Proc. Natl. Acad. Sci. USA (2006) 103 (11), 4005-4010).

ALPHA screening is carried out based on the following principle according to ALPHA technology that uses two beads, a donor and an acceptor. Luminescence signals are detected only when molecules bound with the donor beads interact with molecules bound with the acceptor beads and when the two beads are close to one another. Laser-excited photosensitizers in the donor beads convert ambient oxygen into singlet oxygen in an excited state. The singlet oxygen molecules spread around the donor beads and when they reach the nearby acceptor beads, they induce chemiluminescent reaction in the beads to result in light emission. When the molecule bound with the donor bead and the molecule bound with the acceptor bead do not interact, chemiluminescent reaction does not occur because singlet oxygen produced by the donor bead does not reach the acceptor bead.

For example, a biotin-labeled CXCR3 ligand is bound to the donor bead, and a glutathione S transferase (GST)-tagged CXCR3 peptide is bound to the acceptor bead. The N-terminal extracellular domain region (1^{st} to 53^{rd} amino acids, SEQ ID NO: 89) or a partial fragment thereof (22^{nd} to 42^{nd} amino acids, SEQ ID NO: 90, Biochemistry (2002) 41, 10418-10425) can be used as the CXCR3 peptide. In addition, it is more desirable that the Y residue at position 27 and/or the Y residue at position 29 of CXCR3 have/has undergone sulfation modification (MOLECULAR AND CELLULAR BIOLOGY, August 2006, p. 5838-5849). In the absence of competing untagged CXCR3 ligands, the CXCR3 ligand and CXCR3 peptide interact to produce a signal at 520-620 nm. The untagged CXCR3 ligand competes with the tagged CXCR3 ligand for interaction with the CXCR3 peptide. Decrease in fluorescence resulting from the competition can be quantified to determine relative binding affinity. Biotinylation of a CXCR3 ligand, such as CXCL10, using sulfo-NHS-biotin or the like is known in the art. A method which involves, for example, fusing a polynucleotide encoding the CXCR3 peptide in-frame with a polynucleotide encoding GST to form a fused gene, expressing the GST-fused CXCR3 peptide in cells or the like carrying a vector that permits expression of the fused gene, and purifying the GST-fused CXCR3 peptide using a glutathione column, can be appropriately adopted as a method for tagging a CXCR3 peptide with GST. The obtained signals are preferably analyzed using, for example, the software GRAPHPAD PRISM (GraphPad Software, Inc., San Diego) adapted to a one-site competition model based on non-linear regression analysis.

One (CXCR3 ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other one (analyte, the full-length CXCR3 or the aforementioned CXCR3 peptides can be used as the analyte when fixing a CXCR3 ligand) of the substances between which the interaction is to be observed is poured onto the surface of the sensor chip, and when the analyte binds with the CXCR3 ligand, the mass of the immobilized CXCR3 ligand molecule increases and results in the change of refractive index of the solvent on the sensor chip surface. This change in refractive index shifts the position of the SPR signal (in contrast, the position of the signal returns when dissociation occurs). The Biacore system plots on the ordinate the amount of the above-mentioned shift, i.e., change in mass on the sensor chip surface, and displays time-dependent change in mass as assay data (sensorgram). Kinetics (association rate constant (ka) and dissociation rate constant (kd)) is determined from the curve of the sensorgram, and dissociation constant (KD) is determined from the ratio between the two constants. Inhibition assay or equilibrium analysis is also preferably used in the BIACORE method. Examples of inhibition assay are described in Proc. Natl. Acad. Sci. USA (2006) 103 (11), 4005-4010, and examples of equilibrium analysis are described in Methods Enzymol. 2000; 323:325-40. Using a similar method, the binding of a CXCR3 ligand to CXCR3 can be measured by immobilizing the full-length CXCR3 or the aforementioned CXCR3 peptides on a thin gold film of a sensor chip and pouring the CXCR3 ligand as the analyte. The full-length CXCR3 or the aforementioned CXCR3 peptides immobilized on the thin gold film of the sensor chip may be a purified protein or peptide. Alternatively, a cell expressing them or a cell membrane fraction thereof may also be used.

The CXCR3 ligand of the present disclosure can specifically bind to the full-length CXCR3 or to the aforementioned CXCR3 peptides with a dissociation constant (KD) of 100 μM, 10 μM, 1 μM, 100 nM, 50 nM, 10 nM, 5 nM, 1 nM, 500 pM, 400 pM, 350 pM, 300 pM, 250 pM, 200 pM, 150 pM, 100 pM, 50 pM, 25 pM, 10 pM, 5 pM, 1 pM, 0.5 pM, or 0.1 pM or less.

Alternatively, the binding activity of a CXCR3 ligand to the immobilized full-length CXCR3 or an aforementioned CXCR3 peptide can be evaluated based on the ELISA principle. For example, the full-length CXCR3 or an aforementioned CXCR3 peptide is immobilized in the wells of an ELISA plate. A CXCR3 ligand solution is brought into contact with the immobilized full-length CXCR3 or aforementioned CXCR3 peptide in the wells, and a CXCR3 ligand that binds to the immobilized full-length CXCR3 or aforementioned CXCR3 peptide is detected by an antibody that binds to the CXCR3 ligand. Alternatively, the CXCR3 ligand is immobilized in the wells of an ELISA plate, full-length CXCR3 or aforementioned CXCR3 peptide solution is brought into contact with the immobilized CXCR3 ligand in the wells, and the full-length CXCR3 or an aforementioned CXCR3 peptide that binds to the immobilized CXCR3 ligand is detected by an antibody that binds to the full-length CXCR3 or the aforementioned CXCR3 peptide.

As a method of measuring the binding between a CXCR3 ligand and CXCR3, there is also the method of labelling the CXCR3 ligand using a radioisotope. Specifically, a radioisotope-labeled CXCR3 ligand is prepared, added to CXCR3-expressing cells, and incubated. The incubated sample is passed through a filter to adsorb the CXCR3 ligand bound to CXCR3 onto the filter together with the cells. The amount of CXCR3 ligand adsorbed on the filter can be measured by drying the filter and measuring the radiation amount. There is a report of applying this method to CXCL10 (Molecular and Cellular Biology, August 2006, p. 5838-5849, Vol. 26, No. 15, The Journal of Biological Chemistry Vol. 278, No. 19, Issue of May 9, pp. 17066-17074, 2003).

In addition, by measuring the 15N-1H HSQC NMR spectrum and 2D NOESY NMR spectrum of a 15N-labeled CXCR3 ligand and comparing the NMR spectra obtained before and after the addition of a CXCR3 peptide, residues involved in CXCR3 binding within the CXCR3 ligand and the strength of the bond can also be analyzed (Biochemistry, 2002, 41, 10418-10425).

### CXCR3 Ligands

The term “CXCR3 ligand” as used herein refers to a molecule capable of binding to CXCR3. For example, proteins comprising sites that interact with the ligand-binding site of CXCR3 are included in the CXCR3 ligands of the present disclosure. The term refers to mature CXCR3 ligands that are secreted extracellularly as a result of intracellular processing, and not to “full-length” proteins that have not undergone processing. As used herein, those that have not undergone processing are referred to as CXCR3 ligand precursors.

In a particular embodiment, the CXCR3 ligand provided herein has resistance to DPPIV and the activity of causing migration of cells expressing CXCR3.

DPPIV (Dipeptidyl peptidase IV, Dipeptidyl peptidase 4, DPP4; EC3.4.14.5) is an enzyme (serine protease) that inactivates incretin, which is an intestinal hormone, and is present on the cell membrane as well as in blood as a soluble protein. It is also called adenosine deaminase-binding protein (ADABP) because it also has the function of binding to adenosine deaminase (ADA; EC 3.5.4.4) and modulating intracellular signal transduction. DPPIV has the function of cleaving a dipeptide from a peptide having a proline or alanine residue at the 2nd position from the amino terminal side.

Human DPPIV is a 110 kDa protein composed of 766 amino acids, and its amino acid sequence is shown by Refseq accession number: NP_001926.2.

Evaluation of whether a target protein is cleaved by DPPIV can be done by LC/MS analysis after incubating DPPIV and the target protein. Specifically, when 0.2 mg/ml of the target protein and DPPIV at a final concentration of 200 nM are reacted at 37° C. for 1 hour, mass spectrometry is then done by LC/MS, and the mass of the target protein decreases due to the reaction, this suggests that the target protein was cleaved by DPPIV. In addition, when the mass of the protein is reduced and the mass of the reduced amount and the theoretical mass of the two N-terminal amino acids of the target protein match, this suggests that the two N-terminal amino acids of the target protein were cleaved by DPPIV. When carrying out such a DPPIV cleavage assessment on a CXCR3 ligand of the present disclosure, the protein to be incubated with DPPIV may be the CXCR3 ligand itself, or maybe a fusion protein in which the CXCR3 ligand is fused with another polypeptide (e.g., an antibody Fc region).

“Resistance to DPPIV” in the present disclosure refers to the property of the target protein of not being cleaved by DPPIV. More specifically, it means that the mass of the target protein after DPPIV treatment does not decrease when a cleavage evaluation as described above is carried out. More specifically, it means that the mass reduction of the target protein after DPPIV treatment does not match the theoretical mass of the two N-terminal amino acids of the target protein when a cleavage evaluation as described above is carried out.

The activity of a target protein to cause migration of cells expressing CXCR3 can be measured using a transfectant or cells isolated from a living body which express CXCR3. As an example of a specific method, Ba/F3 transfectant cells expressing mouse CXCR3 (mCXCR3) (hereinafter BaF3/mCXCR3) or Ba/F3 transfectant cells expressing human CXCR3 (hCXCR3) (hereinafter BaF3/hCXCR3) and HTS Transwell™-96 Permeable Supports with 5.0 μm Pore Polycarbonate Membrane (Cat. 3387, Corning) are used, with the target protein as analyte. After adjusting the final concentration of each analyte to be analyzed in solution to a concentration selected from 33 nM, 100 nM, 300 nM, and 1000 nM, 235 μL of each solution is transferred to the lower chamber. Then, BaF3/mCXCR3 or BaF3/hCXCR3 cells are seeded into the upper chamber at 75 μL/well so as to be 2.0×10⁵ cells/well, and the reaction is carried out for 6 hours or 3 hours, under the conditions of 5% carbon dioxide and 37° C. After reacting for 3 hours, 6 hours, or 18 hours, 100 μL of the solution in the lower chamber is transferred to OptiPlate-96 (Cat. 6005299, PerkinElmer) and 100 μL of CellTiter-Glo™ Luminescent Cell Viability Assay solution (Cat. G7571, Promega) is added. After reacting at room temperature for 10 minutes, the luminescence value is measured with a SpectraMax M3 multimode microplate reader (Molecular Devices) to evaluate the level of migration of cells into the lower chamber. The amount of cells that migrated into the lower chamber is reflected by the luminescence intensity.

In a particular embodiment, the activity of a CXCR3 ligand provided herein to cause migration of cells expressing CXCR3 is 30% or more of the activity of naturally-occurring human CXCL10 to cause migration of cells expressing CXCR3. Specifically, it is preferable to include naturally-occurring human CXCL10 as a control in the system for analyzing the activity to cause migration of CXCR3-expressing cells and to compare the fluorescence intensities obtained from the same experiment round of the CXCR3 ligand whose activity is to be compared and the control in order to make the state of the CXCR3 cells used for the measurement uniform. 30% or more of the activity of naturally-occurring human CXCL10 to cause migration of cells expressing CXCR3 does not limit the concentration of the analyte in the system for analyzing the activity to cause migration of cells expressing CXCR3, but it can indicate 30% or more of the activity of naturally-occurring human CXCL10 to cause migration of cells expressing CXCR3 in at least one of the concentrations of 100 nM, 300 nM, and 1000 nM.

When a fusion protein comprising a CXCR3 ligand is used as the analyte for analyzing the activity to cause migration of cells expressing CXCR3, it is preferable that the naturally-occurring human CXCL10 used as the control has a molecular format similar to that of the fusion protein comprising the CXCR3 ligand (i.e., that a control of the fusion protein is prepared by using naturally-occurring human CXCL10 in place of the CXCR3 ligand provided herein).

In a particular embodiment, the activity of the CXCR3 ligand provided herein to cause migration of cells expressing CXCR3 is 25% or more of the activity of naturally-occurring human CXCL11 to cause migration of cells expressing CXCR3. Specifically, it is preferable to include naturally-occurring human CXCL11 as a control in the system for analyzing the activity to cause migration of CXCR3-expressing cells and to compare the fluorescence intensities obtained from the same experiment round of the CXCR3 ligand whose activity is to be compared and the control in order to make the state of the CXCR3 cells used for the measurement uniform. 25% or more of the activity of naturally-occurring human CXCL11 to cause migration of cells expressing CXCR3 does not limit the concentration of the analyte in the system for analyzing the activity to cause migration of cells expressing CXCR3, but it can indicate 25% or more of the activity of naturally-occurring human CXCL11 to cause migration of cells expressing CXCR3 in at least one of the concentrations of 33 nM, 100 nM, and 300 nM.

When a fusion protein comprising a CXCR3 ligand is used as the analyte for analyzing the activity to cause migration of cells expressing CXCR3, it is preferable that the naturally-occurring human CXCL11 used as the control has a molecular format similar to that of the fusion protein comprising the CXCR3 ligand (i.e., that a control of the fusion protein is prepared by using naturally-occurring human CXCL11 in place of the CXCR3 ligand provided herein).

In a particular embodiment, the activity of the CXCR3 ligand provided herein to cause migration of cells expressing CXCR3 is 25% or more of the activity of hITIP to cause migration of cells expressing CXCR3. Specifically, it is preferable to include hITIP as a control in the system for analyzing the activity to cause migration of CXCR3-expressing cells and to compare the fluorescence intensities obtained from the same experiment round of the CXCR3 ligand whose activity is to be compared and the control in order to make the state of the CXCR3 cells used for the measurement uniform. 25% or more of the activity of hITIP to cause migration of cells expressing CXCR3 does not limit the concentration of the analyte in the system for analyzing the activity to cause migration of cells expressing CXCR3, but it can indicate 25% or more of the activity of hITIP to cause migration of cells expressing CXCR3 in at least one of the concentrations of 33 nM, 100 nM, and 300 nM.

When a fusion protein comprising a CXCR3 ligand is used as the analyte for analyzing the activity to cause migration of cells expressing CXCR3, it is preferable that the hITIP used as the control has a molecular format similar to that of the fusion protein comprising the CXCR3 ligand (i.e., that a control of the fusion protein is prepared by using hITIP in place of the CXCR3 ligand provided herein).

In a particular embodiment, the CXCR3 ligand concentration at which the activity of the CXCR3 ligand provided herein to cause migration of cells expressing CXCR3 is at a maximum is lower than the concentration of at least one protein selected from naturally-occurring human CXCL10, naturally-occurring human CXCL11, and hITIP at which the activity to cause migration of cells expressing CXCR3 is at a maximum.

In a particular embodiment, the CXCR3 ligand provided herein does not have P as the 2^{nd} amino acid from the N-terminus. In a particular embodiment, the CXCR3 ligand provided herein does not have A as the 2^{nd} amino acid from the N-terminus.

In a particular embodiment, the CXCR3 ligand provided herein has any of the following sequences (a1) to (a7) at the N-terminus:
(a1) V-X1-L (X1 is F, G, I, K, L, M, T, V, W, or Y);
(a2) X2-V-P (X2 is A, F, G, H, I, K, L, M, N, P, Q, R, S, T, V, W, or Y);
(a3) V-X3-P (X3 is A, F, G, H, I, K, L, M, P, Q, R, S, T, V, W, or Y);
(a4) P-L-S;
(a5) X4-F-P (X4 is A, D, E, F, G, H, I, K, L, M, N, P, Q, S, T, V, W, or Y);
(a6) F-X5-M (X5 is A, D, E, G, H, I, M, N, Q, R, S, T, V, W, or Y); and
(a7) F-X6-P (X6 is A, D, E, F, G, H, L, M, N, P, Q, S, T, W, or Y).

In a particular embodiment, the CXCR3 ligand provided herein has any of the following sequences (b1) to (b7) at the N-terminus:

(b1)
(SEQ ID NO: 205)
V-X1-L-S-R-T-V-R;
(X1 is F, G, I, K, L, M, T, V, W, or Y)
(b2)
(SEQ ID NO: 206)
X2-V-P-L-S-R-T-V-R;
(X2 is A, F, G, H, I, K, L, M, N, P, Q, R,
S, T, V, W, or Y)
(b3)
(SEQ ID NO: 207)
V-X3-P-L-S-R-T-V-R;
(X3 is A, F, G, H, I, K, L, M, P, Q, R, S,
T, V, W, or Y)
(b4)
(SEQ ID NO: 208)
P-L-S-R-T-V-R;
(b5)
(SEQ ID NO: 209)
X4-F-P-M-F-K-R-G-R (X4 is A, D, E, F, G, H,
I, K, L, M, N, P, Q, S, T, V, W, or Y);
(b6)
(SEQ ID NO: 210)
F-X5-M-F-K-R-G-R;
(X5 is A, D, E, G, H, I, M, N, Q, R, S, T,
V, W, or Y)
and
(b7)
(SEQ ID NO: 211)
F-X6-P-M-F-K-R-G-R.
(X6 is A, D, E, F, G, H, L, M, N, P, Q, S,
T, W, or Y)

In a particular embodiment, the CXCR3 ligand provided herein has any of the following sequences (a1) to (a5) at the N-terminus:
(a1) V-X1-L (X1 is F, G, I, L, M, T, V, W, or Y);
(a2) X2-V-P (X2 is A, G, I, L, N, Q, S, T, or W);
(a3) V-X3-P (X3 is A, F, G, I, M, P, T, or V);
(a4) P-L-S; and
(a5) X4-F-P (X4 is A, D, E, G, M, N, Q, S, T, V, or Y).

In a particular embodiment, the CXCR3 ligand provided herein has any of the following sequences (b1) to (b5) at the N-terminus:

(b1)
(SEQ ID NO: 212)
V-X1-L-S-R-T-V-R
(X1 is F, G, I, L, M, T, V, W, or Y);
(b2)
(SEQ ID NO: 213)
X2-V-P-L-S-R-T-V-R
(X2 is A, G, I, L, N, Q, S, T, or W);
(b3)
(SEQ ID NO: 214)
V-X3-P-L-S-R-T-V-R
(X3 is A, F, G, I, M, P, T, or V);
(b4)
(SEQ ID NO: 208)
P-L-S-R-T-V-R;
and
(b5)
(SEQ ID NO: 215)
X4-F-P-M-F-K-R-G-R
(X4 is A, D, E, G, M, N, Q, S, T, V, or Y).

In a particular embodiment, the CXCR3 ligand provided herein has a C-X-C motif. The two cysteines contained in the C-X-C motif can each form disulfide bonds with cysteines other than those contained in the C-X-C motif comprised in the CXCR3 ligand. The C-X-C motif in the CXCR3 ligand can be selected from C-T-C (Cys-Thr-Cys), C-L-C (Cys-Leu-Cys), and C-S-C (Cys-Ser-Cys).

The C-X-C motif in the CXCR3 ligand can be located next to the C-terminus of any of the following N-terminal sequences (b1) to (b7):

(b1)
(SEQ ID NO: 205)
V-X1-L-S-R-T-V-R
(X1 is F, G, I, K, L, M, T, V, W, or Y);
(b2)
(SEQ ID NO: 206)
X2-V-P-L-S-R-T-V-R;
(X2 is A, F, G, H, I, K, L, M, N, P, Q, R,
S, T, V, W, or Y)
(b3)
(SEQ ID NO: 207)
V-X3-P-L-S-R-T-V-R;
(X3 is A, F, G, H, I, K, L, M, P, Q, R, S, T,
V, W, or Y)
(b4)
(SEQ ID NO: 208)
P-L-S-R-T-V-R;
(b5)
(SEQ ID NO: 209)
X4-F-P-M-F-K-R-G-R;
(X4 is A, D, E, F, G, H, I, K, L, M, N, P, Q,
S, T, V, W, or Y)
(b6)
(SEQ ID NO: 210)
F-X5-M-F-K-R-G-R;
(X5 is A, D, E, G, H, I, M, N, Q, R, S, T, V,
W, or Y)
and
(b7)
(SEQ ID NO: 211)
F-X6-P-M-F-K-R-G-R.
(X6 is A, D, E, F, G, H, L, M, N, P, Q, S, T,
W, or Y)

The C-X-C motif in the CXCR3 ligand can be located next to the C-terminus of any of the following sequences (b1) to (b5):

(b1)
(SEQ ID NO: 212)
V-X1-L-S-R-T-V-R;
(X1 is F, G, I, L, M, T, V, W, or Y)
(b2)
(SEQ ID NO: 213)
X2-V-P-L-S-R-T-V-R;
(X2 is A, G, I, L, N, Q, S, T, or W)
(b3)
(SEQ ID NO: 214)
V-X3-P-L-S-R-T-V-R;
(X3 is A, F, G, I, M, P, T, or V)
(b4)
(SEQ ID NO: 208)
P-L-S-R-T-V-R;
and
(b5)
(SEQ ID NO: 215)
X4-F-P-M-F-K-R-G-R.
(X4 is A, D, E, G, M, N, Q, S, T, V, or Y)

In a particular embodiment, the CXCR3 ligand provided herein can further have any of the following (c1) to (c5) at the C-terminus of the C-X-C motif:
(c1) the sequence from the 12th amino acid to the 77th amino acid of SEQ ID NO: 60;
(c2) the sequence from the 12th amino acid to the 73rd amino acid of SEQ ID NO: 61;
(c3) the sequence from the 12th amino acid to the 103rd amino acid of SEQ ID NO: 62;
(c4) the sequence from the 12th amino acid to the 77th amino acid of SEQ ID NO: 1; and
(c5) the sequence from the 12^{th} amino acid to the 77^{th} amino acid of SEQ ID NO: 63.

In a particular embodiment, the CXCR3 ligand provided herein is any of a CXCL10 variant, a CXCL11 variant, a CXCL9 variant, an hITIP variant, and a chimeric protein prepared from those variants.

In a particular embodiment, the CXCR3 ligand provided herein is any of the following sequences (d1) to (d7):
(d1) a sequence shown by any one of SEQ ID NOs: 2 to 57, 92 to 147, and 149 to 204;
(d2) a sequence showing 90% or more sequence identity to SEQ ID NO: 60;
(d3) a sequence showing 90% or more sequence identity to SEQ ID NO: 61;
(d4) a sequence showing 90% or more sequence identity to SEQ ID NO: 62;
(d5) a sequence showing 90% or more sequence identity to SEQ ID NO: 63;
(d6) a sequence showing 90% or more sequence identity to SEQ ID NO: 1; and
(d7) a sequence comprising 10 or less amino acid substitutions, insertions, or deletions to a sequence selected from SEQ ID NOs: 1 to 57, 60 to 63, 92 to 147, and 149 to 204;

In a particular embodiment, the CXCR3 ligand provide herein is a sequence showing 90% or more sequence identity, 95% or more sequence identity, 96% or more sequence identity, 97% or more sequence identity, 98% or more sequence identity, or 99% or more sequence identity to any one of the sequences of SEQ ID NOs: 1 and 60 to 63.

### CXCR3 Ligand Mutants

In certain embodiments, amino acid sequence mutants of the CXCR3 ligands provided herein are contemplated. Amino acid sequence mutants of a CXCR3 ligand may be prepared by introducing appropriate modifications into the nucleotide sequence encoding the CXCR3 ligand, or by peptide synthesis. Such modifications include, for example, deletions from, and/or insertions into and/or substitutions of residues within the amino acid sequences of the CXCR3 ligand. Any combination of deletion, insertion, and substitution can be made to arrive at the final construct, provided that the final construct possesses the desired characteristics (e.g., resistance to DPPIV or activity to cause migration of CXCR3-expressing cells).

### Substitution, Insertion, and Deletion Mutants

In certain embodiments, CXCR3 ligand mutants having one or more amino acid substitutions are provided. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into a CXCR3 ligand of interest and the products screened for a desired activity, e.g., retained/improved resistance to DPPIV or activity to cause migration of CXCR3-expressing cells.

TABLE 1

| Original | | Preferred |
|---|---|---|
| residue | Exemplary substitutions | substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In a particular embodiment, one or more modifications may be carried out regarding the substitutions, insertions, or deletions as long as such modifications do not substantially reduce the abilities of the CXCR3 ligand. In a particular embodiment, the CXCR ligand mutant described above comprises one, two, three, four, five, six, seven, eight, nine, or ten amino acid modifications.

### Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Methods of Conferring Resistance to DPPIV on CXCR3 Ligands

The present disclosure also relates to methods of conferring resistance to DPPIV on a CXCR3 ligand.

In a particular embodiment, the method of conferring resistance to DPPIV on a CXCR3 ligand provided herein comprises modifying the sequence in the vicinity of the N-terminus of a parent CXCR3 ligand.

In a particular embodiment, the 2^{nd} amino acid from the N-terminus of the parent CXCR3 ligand used in the method of conferring resistance to DPPIV on a CXCR3 ligand provided herein is P or A. In a particular embodiment, the 1^{st} amino acid from the N-terminus of the parent CXCR3 ligand used in the method of conferring resistance to DPPIV on a CXCR3 ligand provided herein is V. In a more specific embodiment, the N-terminal sequence of the parent CXCR3 ligand used in the method of conferring resistance to DPPIV on a CXCR3 ligand provided herein is V-P-L or V-A-L. In a more specific embodiment, the N-terminal sequence of the parent CXCR3 ligand used in the method of conferring resistance to DPPIV on a CXCR3 ligand provided herein is V-P-L-S-R-T-V-R (SEQ ID NO: 86) or V-A-L-S-R-T-V-R (SEQ ID NO: 87).

In a particular embodiment, the 2^{nd} amino acid from the N-terminus of the parent CXCR3 ligand used in the method of conferring resistance to DPPIV on a CXCR3 ligand provided herein is P. In a particular embodiment, the 1^{st} amino acid from the N-terminus of the parent CXCR3 ligand used in the method of conferring resistance to DPPIV on a CXCR3 ligand provided herein is F. In a more specific embodiment, the N-terminal sequence of the parent CXCR3 ligand used in the method of conferring resistance to DPPIV on a CXCR3 ligand provided herein is F-P-M. In a more specific embodiment, the N-terminal sequence of the parent CXCR3 ligand used in the method of conferring resistance to DPPIV on a CXCR3 ligand provided herein is F-P-M-F-K-R-G-R (SEQ ID NO: 91).

In a particular embodiment, the parent CXCR3 ligand used in the method of conferring resistance to DPPIV on a CXCR3 ligand provided herein has a C-X-C motif. The two cysteines contained in the C-X-C motif can each form disulfide bonds with cysteines other than those of the C-X-C motif comprised in the parent CXCR3 ligand. The C-X-C motif in the parent CXCR3 ligand can be selected from C-T-C (Cys-Thr-Cys), C-L-C (Cys-Leu-Cys), and C-S-C (Cys-Ser-Cys). In a specific embodiment, the C-X-C motif in the parent CXCR3 ligand can be located next to the C-terminus of the N-terminal sequence V-P-L-S-R-T-V-R (SEQ ID NO: 86) or V-A-L-S-R-T-V-R (SEQ ID NO: 87) or F-P-M-F-K-R-G-R (SEQ ID NO: 91).

In a particular embodiment, the parent CXCR3 ligand used in the method of conferring resistance to DPPIV on a CXCR3 ligand provided herein can further have any of the following (c1) to (c5) at the C-terminus of the C-X-C motif:
(c1) the sequence from the 12^{th} amino acid to the 77^{th} amino acid of SEQ ID NO: 60;
(c2) the sequence from the 12^{th} amino acid to the 73^{rd} amino acid of SEQ ID NO: 61;
(c3) the sequence from the 12^{th} amino acid to the 103^{rd} amino acid of SEQ ID NO: 62;
(c4) the sequence from the 12^{th} amino acid to the 77^{th} amino acid of SEQ ID NO: 1; and
(c5) the sequence from the 12^{th} amino acid to the 77^{th} amino acid of SEQ ID NO: 63.

In a particular embodiment, the parent CXCR3 ligand used in the method of conferring resistance to DPPIV to a CXCR3 ligand provided herein is selected from a naturally-occurring CXCL10, naturally-occurring CXCL11, naturally-occurring CXCL9, CXCL10 variant, CXCL11 variant, CXCL9 variant, and chimeric proteins prepared from them.

In a particular embodiment, the method of conferring resistance to DPPIV on a CXCR3 ligand provided herein includes any of the following:
(1) substituting the 2^{nd} amino acid from the N-terminus of the parent CXCR3 ligand from P or A to F, G, I, K, L, M, T, V, W, or Y;
(2) further adding A, F, G, H, I, K, L, M, N, P, Q, R, S, T, V, W, or Y to the N-terminus of the parent CXCR3 ligand;
(3) inserting A, F, G, H, I, K, L, M, P, Q, R, S, T, V, W, or Y between the 1^{st} and 2^{nd} amino acids at the N-terminus of the parent CXCR3 ligand; and
(4) deleting the V at the N-terminus of the parent CXCR3 ligand.

In other words, the present specification provides the use of any of the following for conferring resistance to DPPIV on a CXCR3 ligand:
(1) substituting the 2^{nd} amino acid from the N-terminus of the parent CXCR3 ligand from P or A to F, G, I, K, L, M, T, V, W, or Y;
(2) further adding A, F, G, H, I, K, L, M, N, P, Q, R, S, T, V, W, or Y to the N-terminus of the parent CXCR3 ligand;
(3) inserting A, F, G, H, I, K, L, M, P, Q, R, S, T, V, W, or Y between the 1^{st} and 2^{nd} amino acids at the N-terminus of the parent CXCR3 ligand; and
(4) deleting the V at the N-terminus of the parent CXCR3 ligand.

In a particular embodiment, the method of conferring resistance to DPPIV on a CXCR3 ligand provided herein includes any of the following:
(1) substituting the 2^{nd} amino acid from the N-terminus of the parent CXCR3 ligand from P or A to F, G, I, L, M, T, V, W, or Y;
(2) further adding A, G, I, L, N, Q, S, T, or W to the N-terminus of the parent CXCR3 ligand;
(3) inserting A, F, G, I, M, P, T, or V between the 1^{st} and 2^{nd} amino acids at the N-terminus of the parent CXCR3 ligand; and
(4) deleting the V at the N-terminus of the parent CXCR3 ligand.

In other words, the present specification provides the use of any of the following for conferring resistance to DPPIV on a CXCR3 ligand:
(1) substituting the 2^{nd} amino acid from the N-terminus of the parent CXCR3 ligand from P or A to F, G, I, L, M, T, V, W, or Y;
(2) further adding A, G, I, L, N, Q, S, T, or W to the N-terminus of the parent CXCR3 ligand;
(3) inserting A, F, G, I, M, P, T, or V between the 1^{st} and 2^{nd} amino acids at the N-terminus of the parent CXCR3 ligand; and
(4) deleting the V at the N-terminus of the parent CXCR3 ligand.

In a particular embodiment, the method of conferring resistance to DPPIV on a CXCR3 ligand provided herein includes any of the following:
(1) further adding A, D, E, F, G, H, I, K, L, M, N, P, Q, S, T, V, W, or Y to the N-terminus of the parent CXCR3 ligand;
(2) substituting the 2^{nd} amino acid from the N-terminus of the parent CXCR3 ligand from P to A, D, E, G, H, I, M, N, Q, R, S, T, V, W, or Y; and
(3) inserting A, D, E, F, G, H, L, M, N, P, Q, S, T, W, or Y between the 1^{st} and 2^{nd} amino acids at the N-terminus of the parent CXCR3 ligand.

In other words, the present specification provides the use of any of the following for conferring resistance to DPPIV on a CXCR3 ligand:
(1) further adding A, D, E, F, G, H, I, K, L, M, N, P, Q, S, T, V, W, or Y to the N-terminus of the parent CXCR3 ligand;
(2) substituting the 2^{nd} amino acid from the N-terminus of the parent CXCR3 ligand from P to A, D, E, G, H, I, M, N, Q, R, S, T, V, W, or Y; and
(3) inserting A, D, E, F, G, H, L, M, N, P, Q, S, T, W, or Y between the 1^{st} and 2^{nd} amino acids at the N-terminus of the parent CXCR3 ligand.

In a particular embodiment, the method of conferring resistance to DPPIV on a CXCR3 ligand provided herein comprises further adding A, D, E, G, M, N, Q, S, T, V, or Y to the N-terminus of the parent CXCR3 ligand.

In other words, the present specification provides the use of further adding A, D, E, G, M, N, Q, S, T, V, or Y to the N-terminus of the parent CXCR3 ligand for conferring resistance to DPPIV on a CXCR3 ligand.

### Fusion Proteins Comprising a CXCR3 Ligand

One aspect of the disclosure relates to fusion proteins comprising a CXCR3 ligand. In a specific embodiment, the fusion proteins of the present disclosure relate to fusion proteins comprising a CXCR3 ligand at the N-terminus. The fusion proteins of the present disclosure may be fusion proteins in which a CXCR3 ligand and an antibody are fused, or may be fusion proteins in which a CXCR3 ligand and an antibody Fc region or another type of protein such as albumin are fused. A specific example includes a fusion protein in which an antibody (including the full-length antibody and antibody fragments), an antibody Fc region, or another type of protein such as albumin is fused to the C-terminus of a CXCR3 ligand. A fusion protein comprising a CXCR3 ligand can be purified using a substance that binds to the fusion protein. For example, when fused with an antibody Fc region, adsorption onto immobilized protein A can be used to recover the CXCR3 ligand.

In the fusion proteins of the present disclosure, the CXCR3 ligand and the fusion partner can be fused via a linker. For example, an arbitrary peptide linker that can be introduced by genetic engineering, or a linker disclosed as a synthetic compound linker (see e.g., Protein Engineering, 9 (3), 299-305, 1996) can be used as the linker used in the fusion of the CXCR3 ligand with the fusion partner.

The length of the peptide linker is not particularly limited and may be appropriately selected by those skilled in the art according to the purpose. Examples of the peptide linker can include, but are not limited to:

Ser
Gly•Ser (GS)
Ser•Gly (SG)
Gly•Gly•Ser (GGS)
Gly•Ser•Gly (GSG)
Ser•Gly•Gly (SGG)
Gly•Ser•Ser (GSS)
Ser•Ser•Gly (SSG)
Ser•Gly•Ser (SGS)
Gly•Gly•Gly•Ser
(GGGS, SEQ ID NO: 64)
Gly•Gly•Ser•Gly
(GGSG, SEQ ID NO: 65)
Gly•Ser•Gly•Gly
(GSGG, SEQ ID NO: 66)
Ser•Gly•Gly•Gly
(SGGG, SEQ ID NO: 67)
Gly•Ser•Ser•Gly
(GSSG, SEQ ID NO: 68)
Gly•Gly•Gly•Gly•Ser
(GGGGS, SEQ ID NO: 69)
Gly•Gly•Gly•Ser•Gly
(GGGSG, SEQ ID NO: 70)
Gly•Gly•Ser•Gly•Gly
(GGSGG, SEQ ID NO: 71)
Gly•Ser•Gly•Gly•Gly
(GSGGG, SEQ ID NO: 72)
Gly•Ser•Gly•Gly•Ser
(GSGGS, SEQ ID NO: 73)
Ser•Gly•Gly•Gly•Gly
(SGGGG, SEQ ID NO: 74)
Gly•Ser•Ser•Gly•Gly
(GSSGG, SEQ ID NO: 75)
Gly•Ser•Gly•Ser•Gly
(GSGSG, SEQ ID NO: 76)
Ser•Gly•Gly•Ser•Gly
(SGGSG, SEQ ID NO: 77)
Gly•Ser•Ser•Ser•Gly
(GSSSG, SEQ ID NO: 78)
Gly•Gly•Gly•Gly•Gly•Ser
(GGGGGS, SEQ ID NO: 79)
Ser•Gly•Gly•Gly•Gly•Gly
(SGGGGG, SEQ ID NO: 80)
Gly•Gly•Gly•Gly•Gly•Gly•Ser
(GGGGGGS, SEQ ID NO: 81)
Ser•Gly•Gly•Gly•Gly•Gly•Gly
(SGGGGGG, SEQ ID NO: 82)
(Gly•Gly•Gly•Gly•Ser (GGGGS, SEQ ID NO: 69))n
(Ser•Gly•Gly•Gly•Gly (SGGGG, SEQ ID NO: 74))n

wherein n is an integer of 1 or larger. However, the length and sequence of the peptide linker can be appropriately selected by those skilled in the art according to the purpose.

The synthetic compound linker (chemical cross-linking agent) is a cross-linking agent usually used in peptide cross-linking, for example, N-hydroxysuccinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), or bis [2-(sulfosuccinimidoxycarbonyloxy)ethyl]sulfone (sulfo-BSOCOES).

These cross-linking agents are commercially available.

### Antibodies

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Antibody Fragments

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

### Fc Regions

The term “Fc region” herein is used to define a C-terminal region of an immunoglobulin heavy chain that contains at least a portion of the constant region. The term includes native sequence Fc regions and mutant Fc regions. In one embodiment, a human IgG heavy chain Fc region extends from Cys226, or from Pro230, to the carboxyl-terminus of the heavy chain. However, the C-terminal lysine (Lys447) or glycine-lysine (Gly446-Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. In the present invention, the Fc region can include various modifications. For example, modifications for increasing the yield of molecules in which Fc is heteroassociated and modifications for suppressing binding to FcγR are known.

### Nucleic Acids/Polynucleotides

An “isolated” nucleic acid/polynucleotide refers to a nucleic acid/polynucleotide molecule that has been separated from a component of its natural environment. An isolated nucleic acid/polynucleotide includes a nucleic acid/polynucleotide molecule contained in cells that ordinarily contain the nucleic acid/polynucleotide molecule, but the nucleic acid/polynucleotide molecule is present extrachromosomally or at a chromosomal location that is different from its natural chromosomal location.

The present disclosure also relates to a nucleic acid/polynucleotide that encodes a CXCR3 ligand, or a nucleic acid/polynucleotide that encodes a fusion protein comprising the CXCR3 ligand.

“Isolated nucleic acid/polynucleotide encoding a CXCR3 ligand” refers to one or more nucleic acid molecules encoding a CXCR3 ligand, including such nucleic acid molecule(s) in a single vector or separate vectors, and such nucleic acid molecule(s) present at one or more locations in a host cell.

### Vectors

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Host Cells

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The present disclosure also relates to host cells comprising a nucleic acid/polynucleotide encoding a CXCR3 ligand or a nucleic acid/polynucleotide encoding a fusion protein comprising the CXCR3 ligand.

### Methods for Producing a CXCR3 Ligand

The polynucleotide according to the present disclosure is usually carried by (or inserted in) an appropriate vector and transfected into host cells. The vector is not particularly limited as long as the vector can stably retain an inserted nucleic acid. For example, when E. coli is used as the host, a pBluescript vector (manufactured by Stratagene Corp.) or the like is preferred as a vector for cloning, although various commercially available vectors can be used.

In the case of using a vector for the purpose of producing a CXCR3 ligand or a fusion protein comprising a CXCR3 ligand of the present disclosure, an expression vector is particularly useful. The expression vector is not particularly limited as long as the vector permits expression of the ligand-binding molecule in vitro, in E. coli, in cultured cells, or in individual organisms. The expression vector is preferably, for example, a pBEST vector (manufactured by Promega Corp.) for in vitro expression, a pET vector (manufactured by Invitrogen Corp.) for expression in E. coli, a pME18S-FL3 vector (GenBank Accession No. AB009864) for expression in cultured cells, and a pME18S vector (Mol Cell Biol. 8:466-472 (1988)) for expression in individual organisms. The insertion of the DNA of the present disclosure into the vector can be performed by a routine method, for example, ligase reaction using restriction sites (Current protocols in Molecular Biology edit. Ausubel et al. (1987) Publish. John Wiley & Sons. Section 11.4-11.11).

The host cells are not particularly limited, and various host cells are used according to the purpose. Examples of the cells for expressing the CXCR3 ligand or the fusion protein may include bacterial cells (e.g., Streptococcus, Staphylococcus, E. coli, Streptomyces, and Bacillus subtilis), fungal cells (e.g., yeasts and Aspergillus), insect cells (e.g., Drosophila S2 and Spodoptera SF9), animal cells (e.g., CHO, COS, HeLa, C127, 3T3, BHK, HEK293, and Bowes melanoma cells) and plant cells. The transfection of the vector to the host cells may be performed by a method known in the art, for example, a calcium phosphate precipitation method, an electroporation method (Current protocols in Molecular Biology edit. Ausubel et al., (1987) Publish. John Wiley & Sons. Section 9.1-9.9), a Lipofectamine method (manufactured by GIBCO-BRL), or a microinjection method.

An appropriate secretory signal can be incorporated into the ligand-binding molecule or the fusion protein of interest, in order to secrete the CXCR3 ligand or the fusion protein expressed in the host cells to the endoplasmic reticulum lumen, periplasmic space, or an extracellular environment. The signal may be endogenous to the ligand-binding molecule or the fusion protein of interest, or may be a foreign signal. Specifically, for example, the signal sequence MNQTAILICCLIFLTLSGIQG (SEQ ID NO: 83), MKKSGVLFLLGIILLVLIGVQG (SEQ ID NO: 84), MSVKGMAIALAVILCATVVQG (SEQ ID NO: 85), or MGWSCIILFLVATATGVHS (SEQ ID NO: 148) can be used.

When the CXCR3 ligand or the fusion protein of the present disclosure is secreted into a medium, the recovery of the CXCR3 ligand or the fusion protein in the above production method is performed by collecting the medium. When the CXCR3 ligand or the fusion protein of the present disclosure is produced in cells, the cells are first lysed and the CXCR3 ligand or the fusion protein is subsequently recovered.

A method known in the art including ammonium sulfate or ethanol precipitation, acid extraction, anion- or cation-exchange chromatography, phosphocellulose chromatography, hydrophobic interaction chromatography, affinity chromatography, hydroxylapatite chromatography, and lectin chromatography can be used for recovering and purifying the CXCR3 ligand or the fusion protein of the present disclosure from the recombinant cell cultures.

The CXCR3 ligands provided herein can also be produced by conferring resistance to DPPIV on a parent CXCR3 ligand. In a particular embodiment, the method of producing a CXCR3 ligand provided herein comprises modifying the sequence near the N-terminus of a parent CXCR3 ligand.

In a particular embodiment, the 2^{nd} amino acid from the N-terminus of a parent CXCR3 ligand used in a method of producing a CXCR3 ligand provided herein is P or A. In a particular embodiment, the 1^{st} amino acid from the N-terminus of a parent CXCR3 ligand used in a method of producing a CXCR3 ligand provided herein is V. In a more specific embodiment, the N-terminal sequence of a parent CXCR3 ligand used in a method of producing a CXCR3 ligand provided herein is V-P-L or V-A-L. In a more specific embodiment, the N-terminal sequence of a parent CXCR3 ligand used in a method of producing a CXCR3 ligand provided herein is V-P-L-S-R-T-V-R (SEQ ID NO: 86) or V-A-L-S-R-T-V-R (SEQ ID NO: 87).

In a particular embodiment, the 2^{nd} amino acid from the N-terminus of a parent CXCR3 ligand used in a method of producing a CXCR3 ligand provided herein is P. In a particular embodiment, the 1^{st} amino acid from the N-terminus of a parent CXCR3 ligand used in a method of producing a CXCR3 ligand provided herein is F. In a more specific embodiment, the N-terminal sequence of a parent CXCR3 ligand used in a method of producing a CXCR3 ligand provided herein is F-P-M. In a more specific embodiment, the N-terminal sequence of a parent CXCR3 ligand used in a method of producing a CXCR3 ligand provided herein is F-P-M-F-K-R-G-R (SEQ ID NO: 91).

In a particular embodiment, the parent CXCR3 ligand used in a method of producing a CXCR3 ligand provided herein has a C-X-C motif. The two cysteines contained in the C—X-C motif can each form disulfide bonds with cysteines other than those of the C-X-C motif comprised in the parent CXCR3 ligand. The C-X-C motif in the parent CXCR3 ligand can be selected from C-T-C (Cys-Thr-Cys), C-L-C (Cys-Leu-Cys), and C-S-C (Cys-Ser-Cys). In a specific embodiment, the CXC motif in the parent CXCR3 ligand can be located next to the C-terminus of the N-terminal sequence V-P-L-S-R-T-V-R (SEQ ID NO: 86) or V-A-L-S-R-T-V-R (SEQ ID NO: 87) or F-P-M-F-K-R-G-R (SEQ ID NO: 91).

In a particular embodiment, the parent CXCR3 ligand used in a method of producing a CXCR3 ligand provided herein may further have at the C-terminus of the C-X-C motif any of the following sequences (c1) to (c5):
(c1) the sequence from the 12^{th} amino acid to the 77^{th} amino acid of SEQ ID NO: 60;
(c2) the sequence from the 12^{th} amino acid to the 73^{rd} amino acid of SEQ ID NO: 61;
(c3) the sequence from the 12^{th} amino acid to the 103^{rd} amino acid of SEQ ID NO: 62;
(c4) the sequence from the 12^{th} amino acid to the 77^{th} amino acid of SEQ ID NO: 1; and
(c5) the sequence from the 12^{th} amino acid to the 77^{th} amino acid of SEQ ID NO: 63.

In a particular embodiment, the parent CXCR3 ligand used in a method of producing a CXCR3 ligand provided herein is selected from a naturally-occurring CXCL10, naturally-occurring CXCL11, naturally-occurring CXCL9, CXCL10 variant, CXCL11 variant, CXCL9 variant, and chimeric proteins prepared from them.

In a particular embodiment, the method of producing a CXCR3 ligand provided herein includes any of the following:
(1) substituting the 2^{nd} amino acid from the N-terminus of the parent CXCR3 ligand from P or A to F, G, I, K, L, M, T, V, W, or Y;
2) further adding A, F, G, H, I, K, L, M, N, P, Q, R, S, T, V, W, or Y to the N-terminus of the parent CXCR3 ligand;
3) inserting A, F, G, H, I, K, L, M, P, Q, R, S, T, V, W, or Y between the 1st and 2nd amino acids at the N-terminus of the parent CXCR3 ligand; and
4) deleting the V at the N-terminus of the parent CXCR3 ligand.

In a particular embodiment, the method of producing a CXCR3 ligand provided herein includes any of the following:
(1) substituting the 2nd amino acid from the N-terminus of the parent CXCR3 ligand from P or A to F, G, I, L, M, T, V, W, or Y;
(2) further adding A, G, I, L, N, Q, S, T, or W to the N-terminus of the parent CXCR3 ligand;
(3) inserting A, F, G, I, M, P, T, or V between the 1st and 2nd amino acids at the N-terminus of the parent CXCR3 ligand; and
(4) deleting the V at the N-terminus of the parent CXCR3 ligand.

In a particular embodiment, the method of producing a CXCR3 ligand provided herein includes any of the following:
(1) further adding A, D, E, F, G, H, I, K, L, M, N, P, Q, S, T, V, W, or Y to the N-terminus of the parent CXCR3 ligand;
(2) substituting the 2^{nd} amino acid from the N-terminus of the parent CXCR3 ligand from P to A, D, E, G, H, I, M, N, Q, R, S, T, V, W, or Y; and
(3) inserting A, D, E, F, G, H, L, M, N, P, Q, S, T, W, or Y between the 1^{st} and 2^{nd} amino acids at the N-terminus of the parent CXCR3 ligand.

In a particular embodiment, the method of producing a CXCR3 ligand provided herein includes further adding A, D, E, G, M, N, Q, S, T, V, or Y to the N-terminus of the parent CXCR3 ligand.

The methods of producing a CXCR3 ligand provided herein may further additionally include, in a specific embodiment, a step of recovering or isolating the CXCR3 ligand containing the above-mentioned modifications.

### Treatments

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the CXCR3 ligands of the present disclosure are used to delay development of a disease or to slow the progression of a disease.

### Pharmaceutical Compositions

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

### Pharmaceutically Acceptable Carriers

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Pharmaceutical Compositions Comprising a CXCR3 Ligand or a Fusion Protein Comprising a CXCR3 Ligand

The present disclosure also relates to pharmaceutical compositions (agents) comprising a CXCR3 ligand of the present disclosure and a pharmaceutically acceptable carrier, and pharmaceutical compositions (agents) comprising a fusion protein comprising a CXCR3 ligand of the present disclosure and a pharmaceutically acceptable carrier.

In the present disclosure, the term “pharmaceutical composition comprising a CXCR3 ligand” may be used interchangeably with a “method for treating a disease, comprising administering a CXCR3 ligand to a subject to be treated” and may be used interchangeably with “use of a CXCR3 ligand for the manufacture of a medicament for treating a disease”. Also, the term “pharmaceutical composition comprising a CXCR3 ligand” may be used interchangeably with “use of a CXCR3 ligand for treating a disease”. The term “pharmaceutical composition comprising a fusion protein comprising a CXCR3 ligand” may be used interchangeably with a “method for treating a disease, comprising administering a fusion protein comprising a CXCR3 ligand to a subject to be treated” and may be used interchangeably with “use of a fusion protein comprising a CXCR3 ligand for the manufacture of a medicament for treating a disease”. Also, the term “pharmaceutical composition comprising a fusion protein comprising a CXCR3 ligand” may be used interchangeably with “use of a fusion protein comprising a CXCR3 ligand for treating a disease”.

In some embodiments of the present disclosure, a composition comprising a CXCR3 ligand can be administered to an individual. In some embodiments of the present disclosure, a fusion protein comprising a CXCR3 ligand can be administered to an individual.

The pharmaceutical composition of the present disclosure can be formulated by use of a method known to those skilled in the art. For example, the pharmaceutical composition can be parenterally used in a form of an injection of a sterile solution or suspension with water or any other pharmaceutically acceptable liquids. The pharmaceutical composition can be formulated, for example, by appropriately combining with a pharmacologically acceptable carrier or medium, specifically, sterile water or physiological saline, a vegetable oil, an emulsifier, a suspending agent, a surfactant, a stabilizer, a flavoring agent, an excipient, a vehicle, an antiseptic, a binder, etc. and mixing them into a unit dosage form required for generally accepted pharmaceutical practice. The amount of the active ingredient in these formulations is set so as to give an appropriate volume in a prescribed range.

A sterile composition for injection can be formulated according to usual pharmaceutical practice using a vehicle such as injectable distilled water. Examples of the injectable aqueous solution include isotonic solutions containing physiological saline, glucose, or other adjuvants (e.g., D-sorbitol, D-mannose, D-mannitol, and sodium chloride). The aqueous solution can be used in combination with an appropriate solubilizer, for example, an alcohol (ethanol, etc.), a polyalcohol (propylene glycol, polyethylene glycol, etc.), or a nonionic surfactant (Polysorbate 80™, HCO-50, etc.).

Examples of the oily liquid include sesame oil and soybean oil, and benzyl benzoate and/or benzyl alcohol can be used in combination as a solubilizer. The oily liquid can be combined with a buffer (e.g., a phosphate buffer solution and a sodium acetate buffer solution), a soothing agent (e.g., procaine hydrochloride), a stabilizer (e.g., benzyl alcohol and phenol), or an antioxidant. The prepared injection solution is usually filled into an appropriate ampule.

The pharmaceutical composition of the present disclosure is preferably administered through a parenteral route. For example, a composition for injection, transnasal administration, transpulmonary administration, or percutaneous administration is administered. The pharmaceutical composition can be administered systemically or locally by, for example, intravenous injection, intramuscular injection, intraperitoneal injection, or subcutaneous injection.

The administration method can be appropriately selected according to the age and symptoms of a patient. The dose of the pharmaceutical composition containing the CXCR3 ligand can be determined to the range of, for example, 0.0001 mg to 1000 mg per kg body weight per dose. Alternatively, the dose can be determined to, for example, 0.001 mg to 100000 mg per patient. However, the present disclosure is not necessarily limited by these numerical values. The dose and the administration method vary depending on the body weight, age, symptoms, and such of a patient, and those skilled in the art can determine an appropriate dose and administration method in consideration of these conditions.

### EXAMPLES

The following are examples of methods and compositions of the present disclosure. It is understood that various other embodiments may be practiced, given the general description provided above.

### Example 1. Construction of Human CXCL10 (hCXCL10) Variants and hCXCL10 Variant Fc Fusions

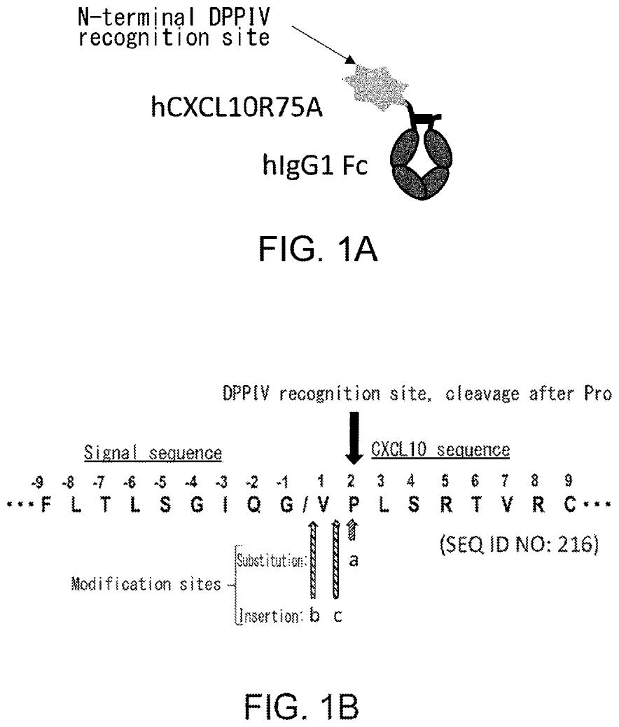

An hCXCL10 variant hCXCL10R75A (SEQ ID NO: 1) in which human CXCL10 (hCXCL10, Refseq: NP_001556.2, Uniprot ID: P02778) has been mutated to be resistant to furin protease, and human CXCL10 variants in which amino acid modifications have been introduced into and around the dipeptidyl peptidase IV (DPPIV) recognition/cleavage site of hCXCL10R75A were prepared. FIG. 1B shows the amino acid modification positions in and around the DPPIV recognition/cleavage site, and Table 2 (Table 2-1 and Table 2-2) shows the names, sequences, and such of the designed human CXCL10 variants.

TABLE 2-1

| | | | Modified, | | | |
|---|---|---|---|---|---|---|
| | | | inserted | | | |
| | | | amino | | | |
| | SEQ | Modifi- | acid | | | |
| Sequence | ID | cation | resi- | Modifi- | N-terminal | |
| Name | NO | pattern | due | cation | sequence | Corresponding hCXCL10 variant Fc fusion |
| hCXCL10R75A | 1 | | | | VPLS (SEQ ID NO: 218) . . . | hCXCL10R75A-G1T4k.one//VHn-G1T4h.one.H435R |
| hCXCL10R75A.0001 | 2 | a | A | P2A | VALS (SEQ ID NO: 219) . . . | hCXCL10R75A.0001-G1T4k.one//VHn-G1T4h.one.H435R |
| hCXCL10R75A.0002 | 3 | a | D | P2D | VDLS (SEQ ID NO: 220) . . . | hCXCL10R75A.0002-G1T4k.one//VHn-G1T4h.one.H435R |
| hCXCL10R75A.0003 | 4 | a | E | P2E | VELS (SEQ ID NO: 221) . . . | hCXCL10R75A.0003-G1T4k.one//VHn-G1T4h.one.H435R |
| hCXCL10R75A.0004 | 5 | a | F | P2F | VFLS (SEQ ID NO: 222) . . . | hCXCL10R75A.0004-G1T4k.one//VHn-G1T4h.one.H435R |
| hCXCL10R75A.0005 | 6 | a | G | P2G | VGLS (SEQ ID NO: 223) . . . | hCXCL10R75A.0005-G1T4k.one//VHn-G1T4h.one.H435R |
| hCXCL10R75A.0006 | 7 | a | H | P2H | VHLS (SEQ ID NO: 224) . . . | hCXCL10R75A.0006-G1T4k.one//VHn-G1T4h.one.H435R |
| hCXCL10R75A.0007 | 8 | a | I | P2I | VILS (SEQ ID NO: 225) . . . | hCXCL10R75A.0007-G1T4k.one//VHn-G1T4h.one.H435R |
| hCXCL10R75A.0008 | 9 | a | K | P2K | VKLS (SEQ ID NO: 226) . . . | hCXCL10R75A.0008-G1T4k.one//VHn-G1T4h.one.H435R |
| hCXCL10R75A.0009 | 10 | a | L | P2L | VLLS (SEQ ID NO: 227) . . . | hCXCL10R75A.0009-G1T4k.one//VHn-G1T4h.one.H435R |
| hCXCL10R75A.0010 | 11 | a | M | P2M | VMLS (SEQ ID NO: 228) . . . | hCXCL10R75A.0010-G1T4k.one//VHn-G1T4h.one.H435R |
| hCXCL10R75A.0011 | 12 | a | N | P2N | VNLS (SEQ ID NO: 229) . . . | hCXCL10R75A.0011-G1T4k.one//VHn-G1T4h.one.H435R |
| hCXCL10R75A.0012 | 13 | a | Q | P2Q | VQLS (SEQ ID NO: 230) . . . | hCXCL10R75A.0012-G1T4k.one//VHn-G1T4h.one.H435R |
| hCXCL10R75A.0013 | 14 | a | R | P2R | VRLS (SEQ ID NO: 231) . . . | hCXCL10R75A.0013-G1T4k.one//VHn-G1T4h.one.H435R |
| hCXCL10R75A.0014 | 15 | a | S | P2S | VSLS (SEQ ID NO: 232) . . . | hCXCL10R75A.0014-G1T4k.one//VHn-G1T4h.one.H435R |
| hCXCL10R75A.0015 | 16 | a | T | P2T | VTLS (SEQ ID NO: 233) . . . | hCXCL10R75A.0015-G1T4k.one//VHn-G1T4h.one.H435R |
| hCXCL10R75A.0016 | 17 | a | V | P2V | VVLS (SEQ ID NO: 234) . . . | hCXCL10R75A.0016-G1T4k.one//VHn-G1T4h.one.H435R |
| hCXCL10R75A.0017 | 18 | a | W | P2W | VWLS (SEQ ID NO: 235) . . . | hCXCL10R75A.0017-G1T4k.one//VHn-G1T4h.one.H435R |
| hCXCL10R75A.0018 | 19 | a | Y | P2Y | VYLS (SEQ ID NO: 236) . . . | hCXCL10R75A.0018-G1T4k.one//VHn-G1T4h.one.H435R |
| hCXCL10R75A.0019 | 20 | b | A | G-1_V1insA | AVPL (SEQ ID NO: 237) . . . | hCXCL10R75A.0019-G1T4k.one//VHn-G1T4h.one.H435R |
| hCXCL10R75A.0020 | 21 | b | D | G-1_V1insD | DVPL (SEQ ID NO: 238) . . . | hCXCL10R75A.0020-G1T4k.one//VHn-G1T4h.one.H435R |
| hCXCL10R75A.0021 | 22 | b | E | G-1_V1insE | EVPL (SEQ ID NO: 239) . . . | hCXCL10R75A.0021-G1T4k.one//VHn-G1T4h.one.H435R |
| hCXCL10R75A.0022 | 23 | b | F | G-1_V1insF | FVPL (SEQ ID NO: 240) . . . | hCXCL10R75A.0022-G1T4k.one//VHn-G1T4h.one.H435R |
| hCXCL10R75A.0023 | 24 | b | G | G-1_V1insG | GVPL (SEQ ID NO: 241) . . . | hCXCL10R75A.0023-G1T4k.one//VHn-G1T4h.one.H435R |
| hCXCL10R75A.0024 | 25 | b | H | G-1_V1insH | HVPL (SEQ ID NO: 242) . . . | hCXCL10R75A.0024-G1T4k.one//VHn-G1T4h.one.H435R |
| hCXCL10R75A.0025 | 26 | b | I | G-1_V1insI | IVPL (SEQ ID NO: 243) . . . | hCXCL10R75A.0025-G1T4k.one//VHn-G1T4h.one.H435R |
| hCXCL10R75A.0026 | 27 | b | K | G-1_V1insK | KVPL (SEQ ID NO: 244) . . . | hCXCL10R75A.0026-G1T4k.one//VHn-G1T4h.one.H435R |
| hCXCL10R75A.0027 | 28 | b | L | G-1_V1insL | LVPL (SEQ ID NO: 245) . . . | hCXCL10R75A.0027-G1T4k.one//VHn-G1T4h.one.H435R |
| hCXCL10R75A.0028 | 29 | b | M | G-1_V1insM | MVPL (SEQ ID NO: 246) . . . | hCXCL10R75A.0028-G1T4k.one//VHn-G1T4h.one.H435R |
| hCXCL10R75A.0029 | 30 | b | N | G-1_V1insN | NVPL (SEQ ID NO: 247) . . . | hCXCL10R75A.0029-G1T4k.one//VHn-G1T4h.one.H435R |

TABLE 2-2

| | | | Modified, | | | |
|---|---|---|---|---|---|---|
| | | | inserted | | | |
| | | | amino | | | |
| | SEQ | Modifi- | acid | | | |
| Sequence | ID | cation | resi- | Modifi- | N-terminal | |
| Name | NO | pattern | due | cation | sequence | Corresponding hCXCL10 variant Fc fusion |
| hCXCL10R75A.0030 | 31 | b | P | G-1_V1insP | PVPL (SEQ ID NO: 248) . . . | hCXCL10R75A.0030-G1T4k.one//VHn-G1T4h.one.H435R |
| hCXCL10R75A.0031 | 32 | b | Q | G-1_V1insQ | QVPL (SEQ ID NO: 249) . . . | hCXCL10R75A.0031-G1T4k.one//VHn-G1T4h.one.H435R |
| hCXCL10R75A.0032 | 33 | b | R | G-1_V1insR | RVPL (SEQ ID NO: 250) . . . | hCXCL10R75A.0032-G1T4k.one//VHn-G1T4h.one.H435R |
| hCXCL10R75A.0033 | 34 | b | S | G-1_V1insS | SVPL (SEQ ID NO: 251) . . . | hCXCL10R75A.0033-G1T4k.one//VHn-G1T4h.one.H435R |
| hCXCL10R75A.0034 | 35 | b | T | G-1_V1insT | TVPL (SEQ ID NO: 252) . . . | hCXCL10R75A.0034-G1T4k.one//VHn-G1T4h.one.H435R |
| hCXCL10R75A.0035 | 36 | b | V | V1dup | VVPL (SEQ ID NO: 253) . . . | hCXCL10R75A.0035-G1T4k.one//VHn-G1T4h.one.H435R |
| | | | | or G-1_ | | |
| | | | | V1insV | | |
| | | | | or V1_ | | |
| | | | | P2insV | | |
| hCXCL10R75A.0036 | 37 | b | W | G-1_ | WVPL (SEQ ID NO: 254) . . . | hCXCL10R75A.0036-G1T4k.one//VHn-G1T4h.one.H435R |
| | | | | V1insW | | |
| hCXCL10R75A.0037 | 38 | b | Y | G-1_V1insY | YVPL (SEQ ID NO: 255) . . . | hCXCL10R75A.0037-G1T4k.one//VHn-G1T4h.one.H435R |
| hCXCL10R75A.0038 | 39 | c | A | V1_P2insA | VAPL (SEQ ID NO: 256) . . . | hCXCL10R75A.0038-G1T4k.one//VHn-G1T4h.one.H435R |
| hCXCL10R75A.0039 | 40 | c | D | V1_P2insD | VDPL (SEQ ID NO: 257) . . . | hCXCL10R75A.0039-G1T4k.one//VHn-G1T4h.one.H435R |
| hCXCL10R75A.0040 | 41 | c | E | V1_P2insE | VEPL (SEQ ID NO: 258) . . . | hCXCL10R75A.0040-G1T4k.one//VHn-G1T4h.one.H435R |
| hCXCL10R75A.0041 | 42 | c | F | V1_P2insF | VFPL (SEQ ID NO: 259) . . . | hCXCL10R75A.0041-G1T4k.one//VHn-G1T4h.one.H435R |
| hCXCL10R75A.0042 | 43 | c | G | V1_P2insG | VGPL (SEQ ID NO: 260) . . . | hCXCL10R75A.0042-G1T4k.one//VHn-G1T4h.one.H435R |
| hCXCL10R75A.0043 | 44 | c | H | V1_P2insH | VHPL (SEQ ID NO: 261) . . . | hCXCL10R75A.0043-G1T4k.one//VHn-G1T4h.one.H435R |
| hCXCL10R75A.0044 | 45 | c | I | V1_P2insl | VIPL (SEQ ID NO: 262) . . . | hCXCL10R75A.0044-G1T4k.one//VHn-G1T4h.one.H435R |
| hCXCL10R75A.0045 | 46 | c | K | V1_P2insK | VKPL (SEQ ID NO: 263) . . . | hCXCL10R75A.0045-G1T4k.one//VHn-G1T4h.one.H435R |
| hCXCL10R75A.0046 | 47 | c | L | V1_P2insL | VLPL (SEQ ID NO: 264) . . . | hCXCL10R75A.0046-G1T4k.one//VHn-G1T4h.one.H435R |
| hCXCL10R75A.0047 | 48 | c | M | V1_P2insM | VMPL (SEQ ID NO: 265) . . . | hCXCL10R75A.0047-G1T4k.one//VHn-G1T4h.one.H435R |
| hCXCL10R75A.0048 | 49 | c | N | V1_P2insN | VNPL (SEQ ID NO: 266) . . . | hCXCL10R75A.0048-G1T4k.one//VHn-G1T4h.one.H435R |
| hCXCL10R75A.0049 | 50 | c | P | P2dup | VPPL (SEQ ID NO: 267) . . . | hCXCL10R75A.0049-G1T4k.one//VHn-G1T4h.one.H435R |
| | | | | or V1_ | | |
| | | | | P2insP | | |
| | | | | or P2_ | | |
| | | | | L3insP | | |
| hCXCL10R75A.0050 | 51 | c | Q | V1_P2insQ | VQPL (SEQ ID NO: 268) . . . | hCXCL10R75A.0050-G1T4k.one//VHn-G1T4h.one.H435R |
| hCXCL10R75A.0051 | 52 | c | R | V1_P2insR | VRPL (SEQ ID NO: 269) . . . | hCXCL10R75A.0051-G1T4k.one//VHn-G1T4h.one.H435R |
| hCXCL10R75A.0052 | 53 | c | S | V1_P2insS | VSPL (SEQ ID NO: 270) . . . | hCXCL10R75A.0052-G1T4k.one//VHn-G1T4h.one.H435R |
| hCXCL10R75A.0053 | 54 | c | T | V1_P2insT | VTPL (SEQ ID NO: 271) . . . | hCXCL10R75A.0053-G1T4k.one//VHn-G1T4h.one.H435R |
| hCXCL10R75A.0054 | 55 | c | V | V1dup | VVPL (SEQ ID NO: 272) . . . | hCXCL10R75A.0054-G1T4k.one//VHn-G1T4h.one.H435R |
| | | | | or G-1_ | | |
| | | | | V1insV | | |
| | | | | or V1_ | | |
| | | | | P2insV | | |
| hCXCL10R75A.0055 | 56 | c | W | V1_P2insW | VWPL (SEQ ID NO: 273) . . . | hCXCL10R75A.0055-G1T4k.one//VHn-G1T4h.one.H435R |
| hCXCL10R75A.0056 | 57 | c | Y | V1_P2insY | VYPL (SEQ ID NO: 274) . . . | hCXCL10R75A.0056-G1T4k.one//VHn-G1T4h.one.H435R |

In order to facilitate the purification of the various hCXCL10 variants, hCXCL10 variant Fc fusions were constructed by fusing the hCXCL10 variants with the human IgG1 antibody Fc domain. A schematic diagram of an hCXCL10 variant Fc fusion is shown in FIG. 1A.

hCXCL10 variant Fc fusions, in which the above hCXCL10 variants are fused with the human IgG1 antibody (hIgG1) Fc domain variant G1T4k.one//VHn-G1T4h.one.H435R (G1T4k.one (SEQ ID NO: 58) and VHn-G1T4h.one.H435R (SEQ ID NO: 59)), were prepared. Specifically, expression vectors encoding genes of peptide chains in which the C-terminus of each hCXCL10 variant and the N-terminus of G1T4k.one in G1T4k.one//VHn-G1T4h.one.H435R are linked were prepared using a method known to those skilled in the art. These peptide chains were combined with VHn-G1T4h.one.H435R, and hCXCL10 variant Fc fusions, in which one hCXCL10 variant is bound to an hIgG1 Fc domain variant, were expressed by transient expression using Expi 293 (Life Technologies) by a method known to those skilled in the art and purified by a method known to those skilled in the art using protein A.

The Fc domain variant used in this study has a mutation for increasing the yield of Fc heteroassociated molecules and a mutation that suppresses FcγR binding.

### Example 2. Evaluation of the Cell Migration-Inducing Activity of hCXCL10 Variant Fc Fusions

Whether the hCXCL10 variant Fc fusions prepared in Example 1 induce migration of cells expressing the CXCL10 receptor (cell migration-inducing activity) was evaluated. hCXCL10R75A was used as a positive control of cell migration-inducing activity. hCXCL10R75A was expressed by transient expression using Expi293 (Life Technologies) by a method known to those skilled in the art, and purified by a method known to those skilled in the art using heparin sepharose (HiTrap Heparin HP Column GE Healthcare), SP sepharose (HiTrap SP HP Column GE Healthcare), and gel filtration (HiLoadSuperdex 75pg GE Healthcare).

Cell migration-inducing activity was evaluated using Ba/F3 transfectant cells expressing mouse CXCR3 (mCXCR3) (hereinafter referred to as BaF3/mCXCR3) and HTS Transwell™-96 Permeable Supports with 5.0 μm Pore Polycarbonate Membrane (Cat. 3387, Corning).

As analytes, hCXCL10 (PeproTech, cat 300-12), hCXCL10R75A, and the following hCXCL10 variant Fc fusions prepared in Example 1 were used:
hCXCL10R75A-G1T4k.one//VHn-G1T4h.one.H435R,
hCXCL10R75A.0001-G1T4k.one//VHn-G1T4h.one.H435R,
hCXCL10R75A.0002-G1T4k.one//VHn-G1T4h.one.H435R,
hCXCL10R75A.0003-G1T4k.one//VHn-G1T4h.one.H435R,
hCXCL10R75A.0004-G1T4k.one//VHn-G1T4h.one.H435R,
hCXCL10R75A.0005-G1T4k.one//VHn-G1T4h.one.H435R,
hCXCL10R75A.0006-G1T4k.one//VHn-G1T4h.one.H435R,
hCXCL10R75A.0007-G1T4k.one//VHn-G1T4h.one.H435R,
hCXCL10R75A.0009-G1T4k.one//VHn-G1T4h.one.H435R,
hCXCL10R75A.0010-G1T4k.one//VHn-G1T4h.one.H435R,
hCXCL10R75A.0011-G1T4k.one//VHn-G1T4h.one.H435R,
hCXCL10R75A.0012-G1T4k.one//VHn-G1T4h.one.H435R,
hCXCL10R75A.0013-G1T4k.one//VHn-G1T4h.one.H435R,
hCXCL10R75A.0014-G1T4k.one//VHn-G1T4h.one.H435R,
hCXCL10R75A.0015-G1T4k.one//VHn-G1T4h.one.H435R,
hCXCL10R75A.0016-G1T4k.one//VHn-G1T4h.one.H435R,
hCXCL10R75A.0017-G1T4k.one//VHn-G1T4h.one.H435R,
hCXCL10R75A.0018-G1T4k.one//VHn-G1T4h.one.H435R,
hCXCL10R75A.0019-G1T4k.one//VHn-G1T4h.one.H435R,
hCXCL10R75A.0020-G1T4k.one//VHn-G1T4h.one.H435R,
hCXCL10R75A.0021-G1T4k.one//VHn-G1T4h.one.H435R,
hCXCL10R75A.0023-G1T4k.one//VHn-G1T4h.one.H435R,
hCXCL10R75A.0025-G1T4k.one//VHn-G1T4h.one.H435R,
hCXCL10R75A.0027-G1T4k.one//VHn-G1T4h.one.H435R,
hCXCL10R75A.0029-G1T4k.one//VHn-G1T4h.one.H435R,
hCXCL10R75A.0031-G1T4k.one//VHn-G1T4h.one.H435R,
hCXCL10R75A.0033-G1T4k.one//VHn-G1T4h.one.H435R,
hCXCL10R75A.0034-G1T4k.one//VHn-G1T4h.one.H435R,
hCXCL10R75A.0036-G1T4k.one//VHn-G1T4h.one.H435R,
hCXCL10R75A.0038-G1T4k.one//VHn-G1T4h.one.H435R,
hCXCL10R75A.0039-G1T4k.one//VHn-G1T4h.one.H435R,
hCXCL10R75A.0040-G1T4k.one//VHn-G1T4h.one.H435R,
hCXCL10R75A.0041-G1T4k.one//VHn-G1T4h.one.H435R,
hCXCL10R75A.0042-G1T4k.one//VHn-G1T4h.one.H435R,
hCXCL10R75A.0044-G1T4k.one//VHn-G1T4h.one.H435R,
hCXCL10R75A.0047-G1T4k.one//VHn-G1T4h.one.H435R,
hCXCL10R75A.0048-G1T4k.one//VHn-G1T4h.one.H435R,
hCXCL10R75A.0049-G1T4k.one//VHn-G1T4h.one.H435R,
hCXCL10R75A.0053-G1T4k.one//VHn-G1T4h.one.H435R, and
hCXCL10R75A.0054-G1T4k.one//VHn-G1T4h.one.H435R.

After adjusting the final concentrations in the solutions of each analyte to be analyzed to 100 nM, 300 nM, and 1000 nM, 235 μL of each solution was transferred to the lower chamber. Then, BaF3/mCXCR3 cells were seeded into the upper chamber at 75 μL/well so as to be 2.0×10⁵ cells/well, and the reaction was carried out for 6 hours or 3 hours. The reaction was carried out under the conditions of 5% carbon dioxide and 37° C. After 3 or 6 hours of reaction, 100 μL of the solution in the lower chamber was transferred to OptiPlate-96 (Cat. 6005299, PerkinElmer) and 100 μL of CellTiter-Glo™ Luminescent Cell Viability Assay solution (Cat. G7571, Promega) was added. After reacting at room temperature for 10 minutes, the luminescence value was measured with a SpectraMax M3 multimode microplate reader (Molecular Devices) to evaluate the level of migration of cells into the lower chamber.

The luminescence intensity reflects the amount of cells that migrated into the lower chamber. Comparison of the cell migration-inducing activities of hCXCL10 (PeproTech, cat 300-12) and hCXCL10R75A and comparison of the cell migration-inducing activities of hCXCL10R75A-G1T4k.one//VHn-G1T4h.one.H435R and other hCXCL10 variant Fc fusions were conducted, respectively.

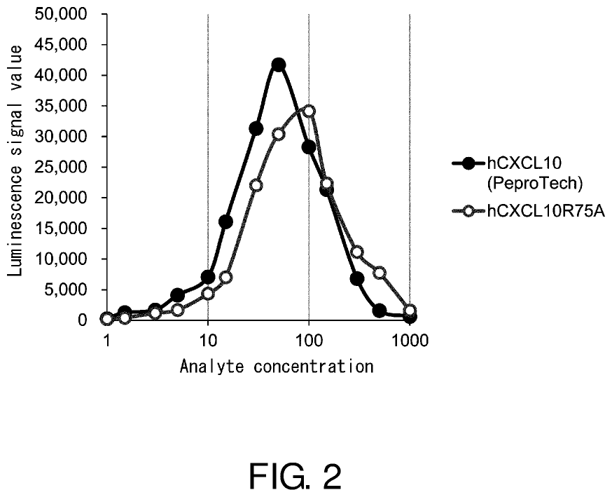

Comparison of the cell migration-inducing activity between hCXCL10 (PeproTech, cat 300-12) and hCXCL10R75A was performed after 6 hours of reaction, and the results are shown in FIG. 2 and Table 3. Both hCXCL10 (PeproTech, cat 300-12) and hCXCL10R75A showed concentration-dependent cell migration-inducing activity, and there was no significant difference in their dependence. From this, it was shown that hCXCL10R75A which was modified to have resistance to furin protease has a similar activity as the wild type.

TABLE 3

| Analyte | Analyte | | Luminescence |
|---|---|---|---|
| name | concentration | | value |
| hCXCL10 | 1 | nM | 257 |
| (PeproTech) | 1.5 | nM | 1252 |
| | 3 | nM | 1677 |
| | 5 | nM | 4118 |
| | 10 | nM | 7094 |
| | 15 | nM | 16110 |
| | 30 | nM | 31321 |
| | 50 | nM | 41726 |
| | 100 | nM | 28288 |
| | 150 | nM | 21343 |
| | 300 | nM | 6822 |
| | 500 | nM | 1587 |
| | 1000 | nM | 634 |
| hCXCL10R75A | 1 | nM | 207 |
| | 1.5 | nM | 389 |
| | 3 | nM | 1185 |
| | 5 | nM | 1677 |
| | 10 | nM | 4354 |
| | 15 | nM | 7024 |
| | 30 | nM | 22026 |
| | 50 | nM | 30392 |
| | 100 | nM | 34130 |
| | 150 | nM | 22345 |
| | 300 | nM | 11163 |
| | 500 | nM | 7751 |
| | 1000 | nM | 1599 |
| cell only | 0 | nM | 275 |

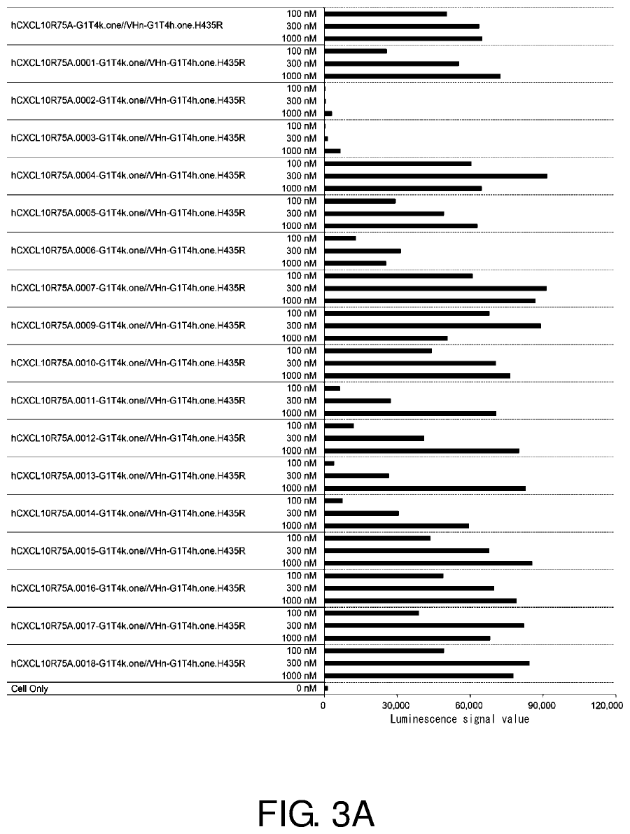

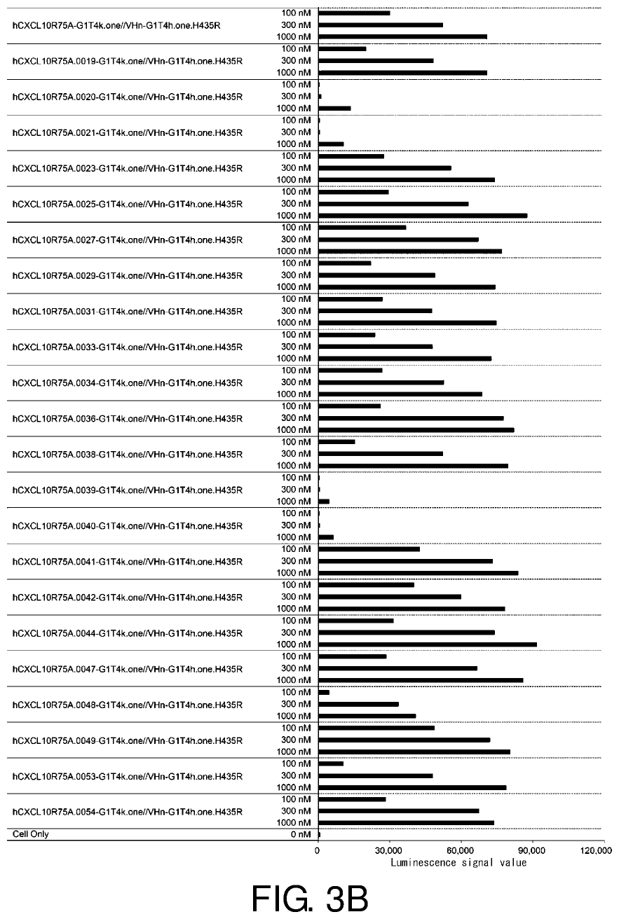

Comparison of the cell migration-inducing activity between hCXCL10R75A-G1T4k.one//VHn-G1T4h.one.H435R and other hCXCL10 variant Fc fusions was performed after 3 hours of reaction, and the results are shown in FIG. 3 and Table 4 (Table 4a, Table 4b-1, and Table 4b-2). Compared to hCXCL10R75A-G1T4k.one//VHn-G1T4h.one.H435R,
hCXCL10R75A.0001-G1T4k.one//VHn-G1T4h.one.H435R,
hCXCL10R75A.0004-G1T4k.one//VHn-G1T4h.one.H435R,
hCXCL10R75A.0005-G1T4k.one//VHn-G1T4h.one.H435R,
hCXCL10R75A.0007-G1T4k.one//VHn-G1T4h.one.H435R,
hCXCL10R75A.0009-G1T4k.one//VHn-G1T4h.one.H435R,
hCXCL10R75A.0010-G1T4k.one//VHn-G1T4h.one.H435R,
hCXCL10R75A.0015-G1T4k.one//VHn-G1T4h.one.H435R,
hCXCL10R75A.0016-G1T4k.one//VHn-G1T4h.one.H435R,
hCXCL10R75A.0017-G1T4k.one//VHn-G1T4h.one.H435R,
hCXCL10R75A.0018-G1T4k.one//VHn-G1T4h.one.H435R,
hCXCL10R75A.0019-G1T4k.one//VHn-G1T4h.one.H435R,
hCXCL10R75A.0023-G1T4k.one//VHn-G1T4h.one.H435R,
hCXCL10R75A.0025-G1T4k.one//VHn-G1T4h.one.H435R,
hCXCL10R75A.0027-G1T4k.one//VHn-G1T4h.one.H435R,
hCXCL10R75A.0029-G1T4k.one//VHn-G1T4h.one.H435R,
hCXCL10R75A.0031-G1T4k.one//VHn-G1T4h.one.H435R,
hCXCL10R75A.0033-G1T4k.one//VHn-G1T4h.one.H435R,
hCXCL10R75A.0034-G1T4k.one//VHn-G1T4h.one.H435R,
hCXCL10R75A.0036-G1T4k.one//VHn-G1T4h.one.H435R,
hCXCL10R75A.0038-G1T4k.one//VHn-G1T4h.one.H435R,
hCXCL10R75A.0041-G1T4k.one//VHn-G1T4h.one.H435R,
hCXCL10R75A.0042-G1T4k.one//VHn-G1T4h.one.H435R,
hCXCL10R75A.0044-G1T4k.one//VHn-G1T4h.one.H435R,
hCXCL10R75A.0047-G1T4k.one//VHn-G1T4h.one.H435R,
hCXCL10R75A.0049-G1T4k.one//VHn-G1T4h.one.H435R,
hCXCL10R75A.0053-G1T4k.one//VHn-G1T4h.one.H435R, and
hCXCL10R75A.0054-G1T4k.one//VHn-G1T4h.one.H435R retained, at each of the concentrations of 100 nM, 300 nM, and 1000 nM, 30% or more of the cell migration-inducing activity of hCXCL10R75A-G1T4k.one//VHn-G1T4h.one.H435R at the same concentration. From this, it was shown that the human CXCL10 variants contained in these human CXCL10 variant Fc fusions have sufficient activity.

TABLE 4a

| | Analyte | Lumines- | Activity |
|---|---|---|---|
| | concen- | cence | retention |
| Analyte name | tration | value | rate* |
| hCXCL10R75A-G1T4k.one//VHn-G1T4h.one.H435R | 100 nM | 50291 | |
| | 300 nM | 63662 | |
| | 1000 nM | 64934 | |
| hCXCL10R75A.0001-G1T4k.one//VHn-G1T4h.one.H435R | 100 nM | 25665 | 51% |
| | 300 nM | 55400 | 87% |
| | 1000 nM | 72476 | 112% |
| hCXCL10R75A.0002-G1T4k.one//VHn-G1T4h.one.H435R | 100 nM | 365 | 1% |
| | 300 nM | 524 | 1% |
| | 1000 nM | 3078 | 5% |
| hCXCL10R75A.0003-G1T4k.one//VHn-G1T4h.one.H435R | 100 nM | 402 | 1% |
| | 300 nM | 1319 | 2% |
| | 1000 nM | 6634 | 10% |
| hCXCL10R75A.0004-G1T4k.one//VHn-G1T4h.one.H435R | 100 nM | 60499 | 120% |
| | 300 nM | 91666 | 144% |
| | 1000 nM | 64738 | 100% |
| hCXCL10R75A.0005-G1T4k.one//VHn-G1T4h.one.H435R | 100 nM | 29314 | 58% |
| | 300 nM | 49215 | 77% |
| | 1000 nM | 62988 | 97% |
| hCXCL10R75A.0006-G1T4k.one//VHn-G1T4h.one.H435R | 100 nM | 13034 | 26% |
| | 300 nM | 31410 | 49% |
| | 1000 nM | 25422 | 39% |
| hCXCL10R75A.0007-G1T4k.one//VHn-G1T4h.one.H435R | 100 nM | 61004 | 121% |
| | 300 nM | 91432 | 144% |
| | 1000 nM | 86763 | 134% |
| hCXCL10R75A.0009-G1T4k.one//VHn-G1T4h.one.H435R | 100 nM | 67910 | 135% |
| | 300 nM | 89121 | 140% |
| | 1000 nM | 50563 | 78% |
| hCXCL10R75A.0010-G1T4k.one//VHn-G1T4h.one.H435R | 100 nM | 44181 | 88% |
| | 300 nM | 70501 | 111% |
| | 1000 nM | 76518 | 118% |
| hCXCLI 0R75A.0011-G1T4k.one//VHn-G1T4h.one.H435R | 100 nM | 6447 | 13% |
| | 300 nM | 27284 | 43% |
| | 1000 nM | 70810 | 109% |
| hCXCL10R75A.0012-G1T4k.one//VHn-G1T4h.one.H435R | 100 nM | 12277 | 24% |
| | 300 nM | 41113 | 65% |
| | 1000 nM | 80105 | 123% |
| hCXCL10R75A.0013-G1T4k.one//VHn-G1T4h.one.H435R | 100 nM | 3951 | 8% |
| | 300 nM | 26601 | 42% |
| | 1000 nM | 82839 | 128% |
| hCXCL10R75A.0014-G1T4k.one//VHn-G1T4h.one.H435R | 100 nM | 7581 | 15% |
| | 300 nM | 30656 | 48% |
| | 1000 nM | 59567 | 92% |
| hCXCL10R75A.0015-G1T4k.one//VHn-G1T4h.one.H435R | 100 nM | 43622 | 87% |
| | 300 nM | 67799 | 106% |
| | 1000 nM | 85480 | 132% |
| hCXCL10R75A.0016-G1T4k.one//VHn-G1T4h.one.H435R | 100 nM | 48930 | 97% |
| | 300 nM | 69864 | 110% |
| | 1000 nM | 79077 | 122% |
| hCXCL10R75A.0017-G1T4k.one//VHn-G1T4h.one.H435R | 100 nM | 38888 | 77% |
| | 300 nM | 82142 | 129% |
| | 1000 nM | 68224 | 105% |
| hCXCL10R75A.0018-G1T4k.one//VHn-G1T4h.one.H435R | 100 nM | 49213 | 98% |
| | 300 nM | 84292 | 132% |
| | 1000 nM | 77813 | 120% |
| cell only | 0 nM | 1348 | — |

Activity retention rate*: percentage to the signal value of hCXCL10R75A-G1T4k.one//VHn-G1T4h.one.H435R at the corresponding concentration

TABLE 4b-1

| | Analyte | Lumines- | Activity |
|---|---|---|---|
| | concen- | cence | retention |
| Analyte name | tration | value | rate* |
| hCXCL10R75A-G1T4k.one//VHn-G1T4h.one.H435R | 100 nM | 30093 | |
| | 300 nM | 52215 | |
| | 1000 nM | 70794 | |
| hCXCL10R75A.0019-G1T4k.one//VHn-G1T4h.one.H435R | 100 nM | 19969 | 66% |
| | 300 nM | 48281 | 92% |
| | 1000 nM | 70794 | 100% |
| hCXCL10R75A.0020-G1T4k.one//VHn-G1T4h.one.H435R | 100 nM | 503 | 2% |
| | 300 nM | 1296 | 2% |
| | 1000 nM | 13592 | 19% |
| hCXCL10R75A.0021-G1T4k.one//VHn-G1T4h.one.H435R | 100 nM | 531 | 2% |
| | 300 nM | 671 | 1% |
| | 1000 nM | 10553 | 15% |
| hCXCL10R75A.0023-G1T4k.one//VHn-G1T4h.one.H435R | 100 nM | 27473 | 91% |
| | 300 nM | 55608 | 106% |
| | 1000 nM | 74001 | 105% |
| hCXCL10R75A.0025-G1T4k.one//VHn-G1T4h.one.H435R | 100 nM | 29422 | 98% |
| | 300 nM | 62870 | 120% |
| | 1000 nM | 87538 | 124% |
| hCXCL10R75A.0027-G1T4k.one//VHn-G1T4h.one.H435R | 100 nM | 36702 | 122% |
| | 300 nM | 67093 | 128% |
| | 1000 nM | 76975 | 109% |
| hCXCL10R75A.0029-G1T4k.one//VHn-G1T4h.one.H435R | 100 nM | 22048 | 73% |
| | 300 nM | 48887 | 94% |
| | 1000 nM | 74244 | 105% |
| hCXCL10R75A.0031-G1T4k.one//VHn-G1T4h.one.H435R | 100 nM | 26942 | 90% |
| | 300 nM | 47666 | 91% |
| | 1000 nM | 74663 | 105% |
| hCXCL10R75A.0033-G1T4k.one//VHn-G1T4h.one.H435R | 100 nM | 23828 | 79% |
| | 300 nM | 47833 | 92% |
| | 1000 nM | 72566 | 103% |
| hCXCL10R75A.0034-G1T4k.one//VHn-G1T4h.one.H435R | 100 nM | 26755 | 89% |
| | 300 nM | 52616 | 101% |
| | 1000 nM | 68706 | 97% |
| hCXCL10R75A.0036-G1T4k.one//VHn-G1T4h.one.H435R | 100 nM | 25990 | 86% |
| | 300 nM | 77812 | 149% |
| | 1000 nM | 82063 | 116% |
| hCXCL10R75A.0038-G1T4k.one//VHn-G1T4h.one.H435R | 100 nM | 15307 | 51% |
| | 300 nM | 52204 | 100% |
| | 1000 nM | 79611 | 112% |
| hCXCL10R75A.0039-G1T4k.one//VHn-G1T4h.one.H435R | 100 nM | 373 | 1% |
| | 300 nM | 559 | 1% |
| | 1000 nM | 4633 | 7% |
| hCXCL10R75A.0040-G1T4k.one//VHn-G1T4h.one.H435R | 100 nM | 336 | 1% |
| | 300 nM | 653 | 1% |
| | 1000 nM | 6339 | 9% |
| hCXCL10R75A.0041-G1T4k.one//VHn-G1T4h.one.H435R | 100 nM | 42584 | 142% |
| | 300 nM | 73123 | 140% |
| | 1000 nM | 83769 | 118% |
| hCXCL10R75A.0042-G1T4k.one//VHn-G1T4h.one.H435R | 100 nM | 40067 | 133% |
| | 300 nM | 59914 | 115% |
| | 1000 nM | 78325 | 111% |
| hCXCL10R75A.0044-G1T4k.one//VHn-G1T4h.one.H435R | 100 nM | 31472 | 105% |
| | 300 nM | 73850 | 141% |
| | 1000 nM | 91553 | 129% |

TABLE 4b-2

| | | | |
|---|---|---|---|
| hCXCL10R75A.0047-G1T4k.one//VHn-G1T4h.one.H435R | 100 nM | 28498 | 95% |
| | 300 nM | 66644 | 128% |
| | 1000 nM | 85941 | 121% |
| hCXCL10R75A.0048-G1T4k.one//VHn-G1T4h.one.H435R | 100 nM | 4559 | 15% |
| | 300 nM | 33606 | 64% |
| | 1000 nM | 40822 | 58% |
| hCXCL10R75A.0049-G1T4k.one//VHn-G1T4h.one.H435R | 100 nM | 48792 | 162% |
| | 300 nM | 71949 | 138% |
| | 1000 nM | 80469 | 114% |
| hCXCL10R75A.0053-G1T4k.one//VHn-G1T4h.one.H435R | 100 nM | 10497 | 35% |
| | 300 nM | 47981 | 92% |
| | 1000 nM | 78791 | 111% |
| hCXCL10R75A.0054-G1T4k.one//VHn-G1T4h.one.H435R | 100 nM | 28405 | 94% |
| | 300 nM | 67371 | 129% |
| | 1000 nM | 73636 | 104% |
| cell only | 0 nM | 792 | — |

Activity retention rate*: percentage to the signal value of hCXCL10R75A-G1T4k.one//VHn-G1T4h.one.H435R at the corresponding concentration

### Example 3. Evaluation of DPPIV Cleavage Resistance of hCXCL10 Variant Fc Fusions

It was examined whether the hCXCL10 variant Fc fusions that induced cell migration in Example 2 are cleaved by DPPIV. Dipeptidyl peptidase IV derived from human (human DPPIV, hDPPIV) (Biolegend, 764102) was used as the protease. Cleavage of the hCXCL10 variant Fc fusions by the protease was evaluated by LC/MS analysis after deglycosylation treatment. After reacting hCXCL10 variant Fc fusions at a final concentration of 0.2 mg/ml and hDPPIV at a final concentration of 200 nM at 37° C. for 1 hour, mass spectrometry by LC/MS was performed by a method known to those skilled in the art, and the cleavage of the hCXCL10 variant Fc fusions was evaluated. For hCXCL10R75A-G1T4k.one//VHn-G1T4h.one.H435R and hCXCL10R75A.0001-G1T4k.one//VHn-G1T4h.one.H435R, protease treatment reduced the mass of the hCXCL10 variant-containing peptide chain in the hCXCL10 variant Fc fusions, and this mass was consistent with the theoretical mass of the sequences of each of the hCXCL10 variant-containing peptide chains lacking the two N-terminal residues. This suggested that the two amino acid residues from the N-terminal of the human CXCL10 variants (hCXCL10R75A, hCXCL10R75A.0001) contained in hCXCL10R75A-G1T4k.one//VHn-G1T4h.one.H435R and hCXCL10R75A.0001-G1T4k.one//VHn-G1T4h.one.H435R were cleaved by hDPPIV.

On the other hand, hCXCL10R75A.0004-G1T4k.one//VHn-G1T4h.one.H435R,
hCXCL10R75A.0005-G1T4k.one//VHn-G1T4h.one.H435R,
hCXCL10R75A.0007-G1T4k.one//VHn-G1T4h.one.H435R,
hCXCL10R75A.0009-G1T4k.one//VHn-G1T4h.one.H435R,
hCXCL10R75A.0010-G1T4k.one//VHn-G1T4h.one.H435R,
hCXCL10R75A.0015-G1T4k.one//VHn-G1T4h.one.H435R,
hCXCL10R75A.0016-G1T4k.one//VHn-G1T4h.one.H435R,
hCXCL10R75A.0017-G1T4k.one//VHn-G1T4h.one.H435R,
hCXCL10R75A.0018-G1T4k.one//VHn-G1T4h.one.H435R,
hCXCL10R75A.0019-G1T4k.one//VHn-G1T4h.one.H435R,
hCXCL10R75A.0023-G1T4k.one//VHn-G1T4h.one.H435R,
hCXCL10R75A.0025-G1T4k.one//VHn-G1T4h.one.H435R,
hCXCL10R75A.0027-G1T4k.one//VHn-G1T4h.one.H435R,
hCXCL10R75A.0029-G1T4k.one//VHn-G1T4h.one.H435R,
hCXCL10R75A.0031-G1T4k.one//VHn-G1T4h.one.H435R,
hCXCL10R75A.0033-G1T4k.one//VHn-G1T4h.one.H435R,
hCXCL10R75A.0034-G1T4k.one//VHn-G1T4h.one.H435R,
hCXCL10R75A.0036-G1T4k.one//VHn-G1T4h.one.H435R,
hCXCL10R75A.0038-G1T4k.one//VHn-G1T4h.one.H435R,
hCXCL10R75A.0041-G1T4k.one//VHn-G1T4h.one.H435R,
hCXCL10R75A.0042-G1T4k.one//VHn-G1T4h.one.H435R,
hCXCL10R75A.0044-G1T4k.one//VHn-G1T4h.one.H435R,
hCXCL10R75A.0047-G1T4k.one//VHn-G1T4h.one.H435R,
hCXCL10R75A.0049-G1T4k.one//VHn-G1T4h.one.H435R,
hCXCL10R75A.0053-G1T4k.one//VHn-G1T4h.one.H435R, and
hCXCL10R75A.0054-G1T4k.one//VHn-G1T4h.one.H435R
   showed no change in mass due to hDPPIV treatment (Table 5). This result suggested that the hCXCL10 variants contained in these hCXCL10 variant Fc fusions exhibit hDPPIV cleavage resistance.

TABLE 5

| | | | Theoretical | Measured | Measured | | |
|---|---|---|---|---|---|---|---|
| | | | mass of | value (Da) | value (Da) | | |
| | | | the fusion | (before | (after | | |
| | | hCXCL10-hIgG1 antibody | protein | DPPIV | DPPIV | Amass | |
| # | Sample name | Fc domain fusion protein name | (Da) | treatment) | treatment) | (Da) | Comment |
| 1 | hCXCL10R75A-G1T4k.one//VHn- | hCXCL10R75A-G1T4k.one | 34627 | 34626 | 34431 | 195 | Loss of the |
| | G1T4h.one.H435R | | | | | | N-terminal |
| | | | | | | | VP was |
| | | | | | | | suggested |
| 2 | hCXCL10R75A.0001-G1T4k.one//VHn- | hCXCL10R75A.0001-G1T4k.one | 34601 | 34601 | 34430 | 171 | Loss of the |
| | G1T4h.one.H435R | | | | | | N-terminal |
| | | | | | | | VA was |
| | | | | | | | suggested |
| 3 | hCXCL10R75A.0004-G1T4k.one//VHn- | hCXCL10R75A.0004-G1T4k.one | 34678 | 34677 | 34676 | 1 | — |
| | G1T4h.one.H435R | | | | | | |
| 4 | hCXCL10R75A.0005-G1T4k.one//VHn- | hCXCL10R75A.0005-G1T4k.one | 34587 | 34586 | 34587 | −1 | — |
| | G1T4h.one.H435R | | | | | | |
| 5 | hCXCL10R75A.0007-G1T4k.one//VHn- | hCXCL10R75A.0007-G1T4k.one | 34644 | 34643 | 34642 | 1 | — |
| | G1T4h.one.H435R | | | | | | |
| 6 | hCXCL10R75A.0009-G1T4k.one//VHn- | hCXCL10R75A.0009-G1T4k.one | 34644 | 34643 | 34642 | 1 | — |
| | G1T4h.one.H435R | | | | | | |
| 7 | hCXCL10R75A.0010-G1T4k.one//VHn- | hCXCL10R75A.0010-G1T4k.one | 34662 | 34661 | 34662 | −1 | — |
| | G1T4h.one.H435R | | | | | | |
| 8 | hCXCL10R75A.0015-G1T4k.one//VHn- | hCXCL10R75A.0015-G1T4k.one | 34631 | 34630 | 34630 | 0 | — |
| | G1T4h.one.H435R | | | | | | |
| 9 | hCXCL10R75A.0016-G1T4k.one//VHn- | hCXCL10R75A.0016-G1T4k.one | 34629 | 34629 | 34628 | 1 | — |
| | G1T4h.one.H435R | | | | | | |
| 10 | hCXCL10R75A.0017-G1T4k.one//VHn- | hCXCL10R75A.0017-G1T4k.one | 34717 | 34716 | 34716 | 0 | — |
| | G1T4h.one.H435R | | | | | | |
| 11 | hCXCL10R75A.0018-G1T4k.one//VHn- | hCXCL10R75A.0018-G1T4k.one | 34694 | 34693 | 34693 | 0 | — |
| | G1T4h.one.H435R | | | | | | |
| 12 | hCXCL10R75A.0019-G1T4k.one//VHn- | hCXCL10R75A.0019-G1T4k.one | 34699 | 34698 | 34697 | 1 | — |
| | G1T4h.one.H435R | | | | | | |
| 13 | hCXCL10R75A.0023-G1T4k.one//VHn- | hCXCL10R75A.0023-G1T4k.one | 34685 | 34684 | 34683 | 1 | — |
| | G1T4h.one.H435R | | | | | | |
| 14 | hCXCL10R75A.0025-G1T4k.one//VHn- | hCXCL10R75A.0025-G1T4k.one | 34741 | 34740 | 34740 | 0 | — |
| | G1T4h.one.H435R | | | | | | |
| 15 | hCXCL10R75A.0027-G1T4k.one//VHn- | hCXCL10R75A.0027-G1T4k.one | 34741 | 34740 | 34739 | 1 | — |
| | G1T4h.one.H435R | | | | | | |
| 16 | hCXCL10R75A.0029-G1T4k.one//VHn- | hCXCL10R75A.0029-G1T4k.one | 34742 | 34741 | 34742 | −1 | — |
| | G1T4h.one.H435R | | | | | | |
| 17 | hCXCL10R75A.0031-G1T4k.one//VHn- | hCXCL10R75A.0031-G1T4k.one | 34739 | 34738 | 34738 | 0 | — |
| | G1T4h.one.H435R | | | | | | |
| 18 | hCXCL10R75A.0033-G1T4k.one//VHn- | hCXCL10R75A.0033-G1T4k.one | 34715 | 34715 | 34713 | 2 | — |
| | G1T4h.one.H435R | | | | | | |
| 19 | hCXCL10R75A.0034-G1T4k.one//VHn- | hCXCL10R75A.0034-G1T4k.one | 34729 | 34728 | 34728 | 0 | — |
| | G1T4h.one.H435R | | | | | | |
| 20 | hCXCL10R75A.0036-G1T4k.one//VHn- | hCXCL10R75A.0036-G1T4k.one | 34814 | 34813 | 34813 | 0 | — |
| | G1T4h.one.H435R | | | | | | |
| 21 | hCXCL10R75A.0038-G1T4k.one//VHn- | hCXCL10R75A.0038-G1T4k.one | 34699 | 34698 | 34698 | 0 | — |
| | G1T4h.one.H435R | | | | | | |
| 22 | hCXCL10R75A.0041-G1T4k.one//VHn- | hCXCL10R75A.0041-G1T4k.one | 34775 | 34774 | 34774 | 0 | — |
| | G1T4h.one.H435R | | | | | | |
| 23 | hCXCL10R75A.0042-G1T4k.one//VHn- | hCXCL10R75A.0042-G1T4k.one | 34685 | 34685 | 34684 | 1 | — |
| | G1T4h.one.H435R | | | | | | |
| 24 | hCXCL10R75A.0044-G1T4k.one//VHn- | hCXCL10R75A.0044-G1T4k.one | 34741 | 34741 | 34741 | 0 | — |
| | G1T4h.one.H435R | | | | | | |
| 25 | hCXCL10R75A.0047-G1T4k.one//VHn- | hCXCL10R75A.0047-G1T4k.one | 34759 | 34758 | 34758 | 0 | — |
| | G1T4h.one.H435R | | | | | | |
| 26 | hCXCL10R75A.0049-G1T4k.one//VHn- | hCXCL10R75A.0049-G1T4k.one | 34725 | 34725 | 34724 | 1 | — |
| | G1T4h.one.H435R | | | | | | |
| 27 | hCXCL10R75A.0053-G1T4k.one//VHn- | hCXCL10R75A.0053-G1T4k.one | 34729 | 34726 | 34728 | −2 | — |
| | G1T4h.one.H435R | | | | | | |
| 28 | hCXCL10R75A.0054-G1T4k.one//VHn- | hCXCL10R75A.0054-G1T4k.one | 34727 | 34727 | 34727 | 0 | — |
| | G1T4h.one.H435R | | | | | | |

Since the two residues from the N-terminal of hCXCL10R75A are cleaved by hDPPIV, it was suggested that the amino acid modifications in and around the DPPIV recognition/cleavage site contained in the above hCXCL10 variants that did not show a mass change due to hDPPIV treatment are useful modification to provide DPPIV resistance to hCXCL10.

### Example 4. Construction of Human CXCL11 (hCXCL11) Variants and hCXCL11 Fc Fusions

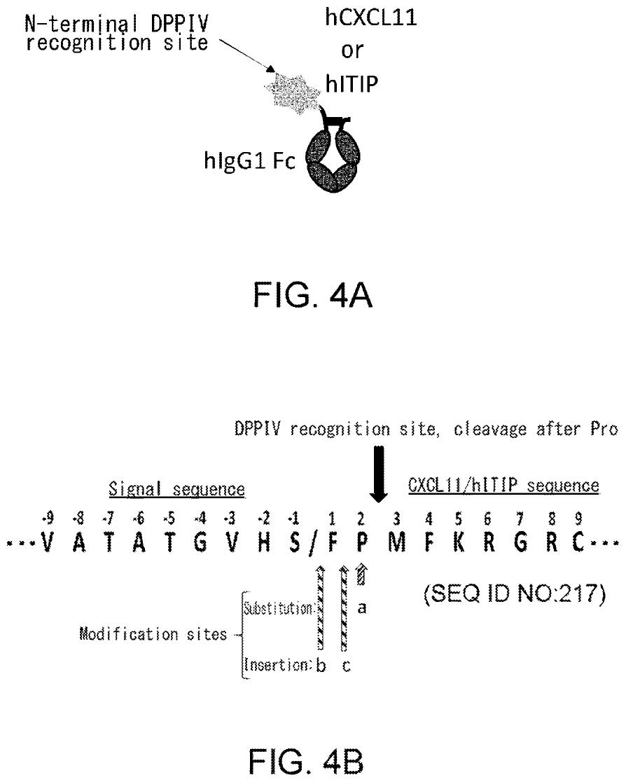

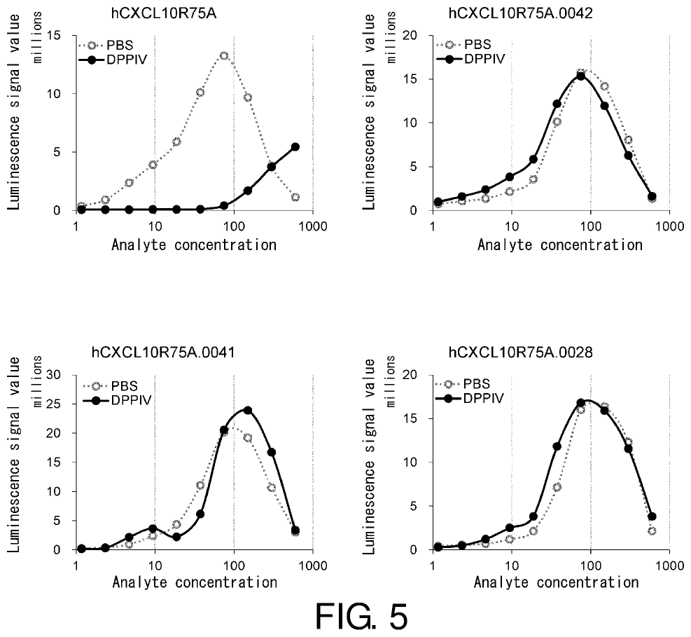

Human CXCL11 (hCXCL11, Refseq: NP_005400.1, Uniprot ID: 014625) (SEQ ID NO: 61) and hCXCL11 variants with amino acid modifications introduced into and around the dipeptidyl peptidase IV (DPPIV) recognition/cleavage site of hCXCL11 were constructed (the amino acid modification positions in and around the DPPIV recognition/cleavage site are shown in FIG. 4B, and the names, sequences, and such of the designed hCXCL11 variants are shown in Table 6).

In order to facilitate the purification of the various hCXCL11 variants, hCXCL11 variant Fc fusions were constructed by fusing the hCXCL11 variants with the human IgG1 antibody Fc domain. A schematic diagram of this is shown in FIG. 4A.

hCXCL11 variant Fc fusions, in which the above hCXCL11 variants are fused with the human IgG1 antibody (hIgG1) Fc domain variant G1T4k.one//VHn-G1T4h.one.H435R (G1T4k.one (SEQ ID NO: 58), VHn-G1T4h.one.H435R (SEQ ID NO: 59)), were prepared. Specifically, expression vectors encoding genes of peptide chains in which the C-terminus of each hCXCL11 variant and the N-terminus of G1T4k.one in G1T4k.one//VHn-G1T4h.one.H435R are linked were prepared using a method known to those skilled in the art. These peptide chains were combined with VHn-G1T4h.one.H435R, and hCXCL11 variant Fc fusions, in which one hCXCL11 variant is bound to an hIgG1 Fc domain variant, were expressed by transient expression using Expi 293 (Life Technologies) by a method known to those skilled in the art and purified by a method known to those skilled in the art using protein A.

The Fc domain variant used in this study has a mutation for increasing the yield of Fc heteroassociated molecules and a mutation that suppresses FcγR binding introduced therein.

TABLE 6

| | | | Modified, | | | |
|---|---|---|---|---|---|---|
| | | | inserted | | | |
| | SEQ | Modifi- | amino | | | |
| Sequence | ID | cation | acid | Modifi- | N-terminal | |
| Name | NO | pattern | residue | cation | sequence | Corresponding hCXCL11 variant Fc fusion |
| hCXCL11 | 61 | | | | FPMF (SEQ ID NO: 275) . . . | hCXCL11-G1T4k.one//VHn-G1T4h.one.H435R |
| hCXCL11.0003 | 92 | a | A | P2A | FAMF (SEQ ID NO: 276) . . . | hCXCL11.0003-G1T4k.one//VHn-G1T4h.one.H435R |
| hCXCL11.0004 | 93 | a | D | P2D | FDMF (SEQ ID NO: 277) . . . | hCXCL11.0004-G1T4k.one//VHn-G1T4h.one.H435R |
| hCXCL11.0005 | 94 | a | E | P2E | FEMF (SEQ ID NO: 278) . . . | hCXCL11.0005-G1T4k.one//VHn-G1T4h.one.H435R |
| hCXCL11.0006 | 95 | a | F | P2F | FFMF (SEQ ID NO: 279) . . . | hCXCL11.0006-G1T4k.one//VHn-G1T4h.one.H435R |
| hCXCL11.0007 | 96 | a | G | P2G | FGMF (SEQ ID NO: 280) . . . | hCXCL11.0007-G1T4k.one//VHn-G1T4h.one.H435R |
| hCXCL11.0008 | 97 | a | H | P2H | FHMF (SEQ ID NO: 281) . . . | hCXCL11.0008-G1T4k.one//VHn-G1T4h.one.H435R |
| hCXCL11.0009 | 98 | a | I | P2I | FIMF (SEQ ID NO: 282) . . . | hCXCL11.0009-G1T4k.one//VHn-G1T4h.one.H435R |
| hCXCL11.0010 | 99 | a | K | P2K | FKMF (SEQ ID NO: 283) . . . | hCXCL11.0010-G1T4k.one//VHn-G1T4h.one.H435R |
| hCXCL11.0011 | 100 | a | L | P2L | FLMF (SEQ ID NO: 284) . . . | hCXCL11.0011-G1T4k.one//VHn-G1T4h.one.H435R |
| hCXCL11.0012 | 101 | a | M | P2M | FMMF (SEQ ID NO: 285) . . . | hCXCL11.0012-G1T4k.one//VHn-G1T4h.one.H435R |
| hCXCL11.0013 | 102 | a | N | P2N | FNMF (SEQ ID NO: 286) . . . | hCXCL11.0013-G1T4k.one//VHn-G1T4h.one.H435R |
| hCXCL11.0014 | 103 | a | Q | P2Q | FQMF (SEQ ID NO: 287) . . . | hCXCL11.0014-G1T4k.one//VHn-G1T4h.one.H435R |
| hCXCL11.0015 | 104 | a | R | P2R | FRMF (SEQ ID NO: 288) . . . | hCXCL11.0015-G1T4k.one//VHn-G1T4h.one.H435R |
| hCXCL11.0016 | 105 | a | S | P2S | FSMF (SEQ ID NO: 289) . . . | hCXCL11.0016-G1T4k.one//VHn-G1T4h.one.H435R |
| hCXCL11.0017 | 106 | a | T | P2T | FTMF (SEQ ID NO: 290) . . . | hCXCL11.0017-G1T4k.one//VHn-G1T4h.one.H435R |
| hCXCL11.0018 | 107 | a | V | P2V | FVMF (SEQ ID NO: 291) . . . | hCXCL11.0018-G1T4k.one//VHn-G1T4h.one.H435R |
| hCXCL11.0019 | 108 | a | W | P2W | FWMF (SEQ ID NO: 292) . . . | hCXCL11.0019-G1T4k.one//VHn-G1T4h.one.H435R |
| hCXCL11.0020 | 109 | a | Y | P2Y | FYMF (SEQ ID NO: 293) . . . | hCXCL11.0020-G1T4k.one//VHn-G1T4h.one.H435R |
| hCXCL11.0021 | 110 | b | A | S-1_F1insA | AFPM (SEQ ID NO: 294) . . . | hCXCL11.0021-G1T4k.one//VHn-G1T4h.one.H435R |
| hCXCL11.0022 | 111 | b | D | S-1_F1insD | DFPM (SEQ ID NO: 295) . . . | hCXCL11.0022-G1T4k.one//VHn-G1T4h.one.H435R |
| hCXCL11.0023 | 112 | b | E | S-1_F1insE | EFPM (SEQ ID NO: 296) . . . | hCXCL11.0023-G1T4k.one//VHn-G1T4h.one.H435R |
| hCXCL11.0024 | 113 | b | F | S-1_F1insF | FFPM (SEQ ID NO: 297) . . . | hCXCL11.0024-G1T4k.one//VHn-G1T4h.one.H435R |
| hCXCL11.0025 | 114 | b | G | S-1_F1insG | GFPM (SEQ ID NO: 298) . . . | hCXCL11.0025-G1T4k.one//VHn-G1T4h.one.H435R |
| hCXCL11.0026 | 115 | b | H | S-1_F1insH | HFPM (SEQ ID NO: 299) . . . | hCXCL11.0026-G1T4k.one//VHn-G1T4h.one.H435R |
| hCXCL11.0027 | 116 | b | I | S-1_F1insI | IFPM (SEQ ID NO: 300) . . . | hCXCL11.0027-G1T4k.one//VHn-G1T4h.one.H435R |
| hCXCL11.0028 | 117 | b | K | S-1_F1insK | KFPM (SEQ ID NO: 301) . . . | hCXCL11.0028-G1T4k.one//VHn-G1T4h.one.H435R |
| hCXCL11.0029 | 118 | b | L | S-1_F1insL | LFPM (SEQ ID NO: 302) . . . | hCXCL11.0029-G1T4k.one//VHn-G1T4h.one.H435R |
| hCXCL11.0030 | 119 | b | M | S-1_F1insM | MFPM (SEQ ID NO: 303) . . . | hCXCL11.0030-G1T4k.one//VHn-G1T4h.one.H435R |
| hCXCL11.0031 | 120 | b | N | S-1_F1insN | NFPM (SEQ ID NO: 304) . . . | hCXCL11.0031-G1T4k.one//VHn-G1T4h.one.H435R |
| hCXCL11.0032 | 121 | b | P | S-1_F1insP | PFPM (SEQ ID NO: 305) . . . | hCXCL11.0032-G1T4k.one//VHn-G1T4h.one.H435R |
| hCXCL11.0033 | 122 | b | Q | S-1_F1insQ | QFPM (SEQ ID NO: 306) . . . | hCXCL11.0033-G1T4k.one//VHn-G1T4h.one.H435R |
| hCXCL11.0034 | 123 | b | R | S-1_F1insR | RFPM (SEQ ID NO: 307) . . . | hCXCL11.0034-G1T4k.one//VHn-G1T4h.one.H435R |
| hCXCL11.0035 | 124 | b | S | S-1_F1insS | SFPM (SEQ ID NO: 308) . . . | hCXCL11.0035-G1T4k.one//VHn-G1T4h.one.H435R |
| hCXCL11.0036 | 125 | b | T | S-1_F1insT | TFPM (SEQ ID NO: 309) . . . | hCXCL11.0036-G1T4k.one//VHn-G1T4h.one.H435R |
| hCXCL11.0037 | 126 | b | V | S-1_F1insV | VFPM (SEQ ID NO: 310) . . . | hCXCL11.0037-G1T4k.one//VHn-G1T4h.one.H435R |
| hCXCL11.0038 | 127 | b | W | S-1_F1insW | WFPM (SEQ ID NO: 311) . . . | hCXCL11.0038-G1T4k.one//VHn-G1T4h.one.H435R |
| hCXCL11.0039 | 128 | b | Y | S-1_F1insY | YFPM (SEQ ID NO: 312) . . . | hCXCL11.0039-G1T4k.one//VHn-G1T4h.one.H435R |
| hCXCL11.0040 | 129 | c | A | F1_P2insA | FAPM (SEQ ID NO: 313) . . . | hCXCL11.0040-G1T4k.one//VHn-G1T4h.one.H435R |
| hCXCL11.0041 | 130 | c | D | F1_P2insD | FDPM (SEQ ID NO: 314) . . . | hCXCL11.0041-G1T4k.one//VHn-G1T4h.one.H435R |
| hCXCL11.0042 | 131 | c | E | F1_P2insE | FEPM (SEQ ID NO: 315) . . . | hCXCL11.0042-G1T4k.one//VHn-G1T4h.one.H435R |
| hCXCL11.0043 | 132 | c | F | F1_P2insF | FFPM (SEQ ID NO: 316) . . . | hCXCL11.0043-G1T4k.one//VHn-G1T4h.one.H435R |
| hCXCL11.0044 | 133 | c | G | F1_P2insG | FGPM (SEQ ID NO: 317) . . . | hCXCL11.0044-G1T4k.one//VHn-G1T4h.one.H435R |
| hCXCL11.0045 | 134 | c | H | F1_P2insH | FHPM (SEQ ID NO: 318) . . . | hCXCL11.0045-G1T4k.one//VHn-G1T4h.one.H435R |
| hCXCL11.0046 | 135 | c | I | F1_P2insI | FIPM (SEQ ID NO: 319) . . . | hCXCL11.0046-G1T4k.one//VHn-G1T4h.one.H435R |
| hCXCL11.0047 | 136 | c | K | F1_P2insK | FKPM (SEQ ID NO: 320) . . . | hCXCL11.0047-G1T4k.one//VHn-G1T4h.one.H435R |
| hCXCL11.0048 | 137 | c | L | F1_P2insL | FLPM (SEQ ID NO: 321) . . . | hCXCL11.0048-G1T4k.one//VHn-G1T4h.one.H435R |
| hCXCL11.0049 | 138 | c | M | F1_P2insM | FMPM (SEQ ID NO: 322) . . . | hCXCL11.0049-G1T4k.one//VHn-G1T4h.one.H435R |
| hCXCL11.0050 | 139 | c | N | F1_P2insN | FNPM (SEQ ID NO: 323) . . . | hCXCL11.0050-G1T4k.one//VHn-G1T4h.one.H435R |
| hCXCL11.0051 | 140 | c | P | F1_P2insP | FPPM (SEQ ID NO: 324) . . . | hCXCL11.0051-G1T4k.one//VHn-G1T4h.one.H435R |
| hCXCL11.0052 | 141 | c | Q | F1_P2insQ | FQPM (SEQ ID NO: 325) . . . | hCXCL11.0052-G1T4k.one//VHn-G1T4h.one.H435R |
| hCXCL11.0053 | 142 | c | R | F1_P2insR | FRPM (SEQ ID NO: 326) . . . | hCXCL11.0053-G1T4k.one//VHn-G1T4h.one.H435R |
| hCXCL11.0054 | 143 | c | S | F1_P2insS | FSPM (SEQ ID NO: 327) . . . | hCXCL11.0054-G1T4k.one//VHn-G1T4h.one.H435R |
| hCXCL11.0055 | 144 | c | T | F1_P2insT | FTPM (SEQ ID NO: 328) . . . | hCXCL11.0055-G1T4k.one//VHn-G1T4h.one.H435R |
| hCXCL11.0056 | 145 | c | V | F1_P2insV | FVPM (SEQ ID NO: 329) . . . | hCXCL11.0056-G1T4k.one//VHn-G1T4h.one.H435R |
| hCXCL11.0057 | 146 | c | W | F1_P2insW | FWPM (SEQ ID NO: 330) . . . | hCXCL11.0057-G1T4k.one//VHn-G1T4h.one.H435R |
| hCXCL11.0058 | 147 | c | Y | F1_P2insY | FYPM (SEQ ID NO: 331) . . . | hCXCL11.0058-G1T4k.one//VHn-G1T4h.one.H435R |

### Example 5. Construction of Human CXCL10-Human CXCL11 Chimeric Protein (hITIP) and hITIP Fc Fusions

Human CXCL10-human CXCL11 chimeric protein (hITIP, SEQ ID NO: 63), in which the 1^{st} to 24^{th} amino acid residues of hCXCL11 (SEQ ID NO: 61) and the 25^{th} to 77^{th} amino acid residues of an hCXCL10 variant (SEQ ID NO: 1) are bound, and hITIP variants in which amino acid modifications were introduced into and around the dipeptidyl peptidase IV (DPPIV) recognition/cleavage site of hITIP were constructed (the amino acid modification positions in and around the DPPIV recognition/cleavage site is shown in FIG. 4B, and the names, sequences, and such of the designed hITIP variants are shown in Table 7).

In order to facilitate the purification of the various hITIP variants, hITIP variant Fc fusions were constructed by fusing the hITIP variants with the human IgG1 antibody Fc domain. A schematic diagram of this is shown in FIG. 4A.

hITIP variant Fc fusions, in which the above hITIP variants are fused with the human IgG1 antibody (hIgG1) Fc domain variant G1T4k.one//VHn-G1T4h.one.H435R (G1T4k.one (SEQ ID NO: 58), VHn-G1T4h.one.H435R (SEQ ID NO: 59)), were prepared. Specifically, expression vectors encoding genes of peptide chains in which the C-terminus of each hITIP variant and the N-terminus of G1T4k.one in G1T4k.one//VHn-G1T4h.one.H435R are linked were prepared by a method known to those skilled in the art. These peptide chains were combined with VHn-G1T4h.one.H435R, and hITIP variant Fc fusions, in which one hITIP variant is bound to an hIgG1 Fc domain variant, were expressed by transient expression using Expi 293 (Life Technologies) by a method known to those skilled in the art and purified by a method known to those skilled in the art using protein A.

The Fc domain variant used in this study has a mutation for increasing the yield of Fc heteroassociated molecules and a mutation that suppresses FcγR binding introduced therein.

TABLE 7

| | | | Modified, | | | |
|---|---|---|---|---|---|---|
| | | | inserted | | | |
| | SEQ | Modifi- | amino | | | |
| Sequence | ID | cation | acid | Modifi- | N-terminal | |
| Name | NO | pattern | residue | cation | sequence | Corresponding hITIP variant Fc fusion |
| hITIP | 63 | | | | FPMF (SEQ ID NO: 332) . . . | hITIP-G1T4k.one//VHn-G1T4h.one.H435R |
| hITIP0003 | 149 | a | A | P2A | FAMF (SEQ ID NO: 333) . . . | hITIP0003-G1T4k.one//VHn-G1T4h.one.H435R |
| hITIP0004 | 150 | a | D | P2D | FDMF (SEQ ID NO: 334) . . . | hITIP0004-G1T4k.one//VHn-G1T4h.one.H435R |
| hITIP0005 | 151 | a | E | P2E | FEMF (SEQ ID NO: 335) . . . | hITIP0005-G1T4k.one//VHn-G1T4h.one.H435R |
| hITIP0006 | 152 | a | F | P2F | FFMF (SEQ ID NO: 336) . . . | hITIP0006-G1T4k.one//VHn-G1T4h.one.H435R |
| hITIP0007 | 153 | a | G | P2G | FGMF (SEQ ID NO: 337) . . . | hITIP0007-G1T4k.one//VHn-G1T4h.one.H435R |
| hITIP0008 | 154 | a | H | P2H | FHMF (SEQ ID NO: 338) . . . | hITIP0008-G1T4k.one//VHn-G1T4h.one.H435R |
| hITIP0009 | 155 | a | I | P2I | FIMF (SEQ ID NO: 339) . . . | hITIP0009-G1T4k.one//VHn-G1T4h.one.H435R |
| hITIP0010 | 156 | a | K | P2K | FKMF (SEQ ID NO: 340) . . . | hITIP0010-G1T4k.one//VHn-G1T4h.one.H435R |
| hITIP0011 | 157 | a | L | P2L | FLMF (SEQ ID NO: 341) . . . | hITIP0011-G1T4k.one//VHn-G1T4h.one.H435R |
| hITIP0012 | 158 | a | M | P2M | FMMF (SEQ ID NO: 342) . . . | hITIP0012-G1T4k.one//VHn-G1T4h.one.H435R |
| hITIP0013 | 159 | a | N | P2N | FNMF (SEQ ID NO: 343) . . . | hITIP0013-G1T4k.one//VHn-G1T4h.one.H435R |
| hITIP0014 | 160 | a | Q | P2Q | FQMF (SEQ ID NO: 344) . . . | hITIP0014-G1T4k.one//VHn-G1T4h.one.H435R |
| hITIP0015 | 161 | a | R | P2R | FRMF (SEQ ID NO: 345) . . . | hITIP0015-G1T4k.one//VHn-G1T4h.one.H435R |
| hITIP0016 | 162 | a | S | P2S | FSMF (SEQ ID NO: 346) . . . | hITIP0016-G1T4k.one//VHn-G1T4h.one.H435R |
| hITIP0017 | 163 | a | T | P2T | FTMF (SEQ ID NO: 347) . . . | hITIP0017-G1T4k.one//VHn-G1T4h.one.H435R |
| hITIP0018 | 164 | a | V | P2V | FVMF (SEQ ID NO: 348) . . . | hITIP0018-G1T4k.one//VHn-G1T4h.one.H435R |
| hITIP0019 | 165 | a | W | P2W | FWMF (SEQ ID NO: 349) . . . | hITIP0019-G1T4k.one//VHn-G1T4h.one.H435R |
| hITIP0020 | 166 | a | Y | P2Y | FYMF (SEQ ID NO: 350) . . . | hITIP0020-G1T4k.one//VHn-G1T4h.one.H435R |
| hITIP0021 | 167 | b | A | S-1_F1insA | AFPM (SEQ ID NO: 351) . . . | hITIP0021-G1T4k.one//VHn-G1T4h.one.H435R |
| hITIP0022 | 168 | b | D | S-1_F1insD | DFPM (SEQ ID NO: 352) . . . | hITIP0022-G1T4k.one//VHn-G1T4h.one.H435R |
| hITIP0023 | 169 | b | E | S-1_F1insE | EFPM (SEQ ID NO: 353) . . . | hITIP0023-G1T4k.one//VHn-G1T4h.one.H435R |
| hITIP0024 | 170 | b | F | S-1_F1insF | FFPM (SEQ ID NO: 354) . . . | hITIP0024-G1T4k.one//VHn-G1T4h.one.H435R |
| hITIP0025 | 171 | b | G | S-1_F1insG | GFPM (SEQ ID NO: 355) . . . | hITIP0025-G1T4k.one//VHn-G1T4h.one.H435R |
| hITIP0026 | 172 | b | H | S-1_F1insH | HFPM (SEQ ID NO: 356) . . . | hITIP0026-G1T4k.one//VHn-G1T4h.one.H435R |
| hITIP0027 | 173 | b | I | S-1_F1insI | IFPM (SEQ ID NO: 357) . . . | hITIP0027-G1T4k.one//VHn-G1T4h.one.H435R |
| hITIP0028 | 174 | b | K | S-1_F1insK | KFPM (SEQ ID NO: 358) . . . | hITIP0028-G1T4k.one//VHn-G1T4h.one.H435R |
| hITIP0029 | 175 | b | L | S-1_F1insL | LFPM (SEQ ID NO: 359) . . . | hITIP0029-G1T4k.one//VHn-G1T4h.one.H435R |
| hITIP0030 | 176 | b | M | S-1_F1insM | MFPM (SEQ ID NO: 360) . . . | hITIP0030-G1T4k.one//VHn-G1T4h.one.H435R |
| hITIP0031 | 177 | b | N | S-1_F1insN | NFPM (SEQ ID NO: 361) . . . | hITIP0031-G1T4k.one//VHn-G1T4h.one.H435R |
| hITIP0032 | 178 | b | P | S-1_F1insP | PFPM (SEQ ID NO: 362) . . . | hITIP0032-G1T4k.one//VHn-G1T4h.one.H435R |
| hITIP0033 | 179 | b | Q | S-1_F1insQ | QFPM (SEQ ID NO: 363) . . . | hITIP0033-G1T4k.one//VHn-G1T4h.one.H435R |
| hITIP0034 | 180 | b | R | S-1_F1insR | RFPM (SEQ ID NO: 364) . . . | hITIP0034-G1T4k.one//VHn-G1T4h.one.H435R |
| hITIP0035 | 181 | b | S | S-1_F1insS | SFPM (SEQ ID NO: 365) . . . | hITIP0035-G1T4k.one//VHn-G1T4h.one.H435R |
| hITIP0036 | 182 | b | T | S-1_F1insT | TFPM (SEQ ID NO: 366) . . . | hITIP0036-G1T4k.one//VHn-G1T4h.one.H435R |
| hITIP0037 | 183 | b | V | S-1_F1insV | VFPM (SEQ ID NO: 367) . . . | hITIP0037-G1T4k.one//VHn-G1T4h.one.H435R |
| hITIP0038 | 184 | b | W | S-1_F1insW | WFPM (SEQ ID NO: 368) . . . | hITIP0038-G1T4k.one//VHn-G1T4h.one.H435R |
| hITIP0039 | 185 | b | Y | S-1_F1insY | YFPM (SEQ ID NO: 369) . . . | hITIP0039-G1T4k.one//VHn-G1T4h.one.H435R |
| hITIP0040 | 186 | c | A | F1_P2insA | FAPM (SEQ ID NO: 370) . . . | hITIP0040-G1T4k.one//VHn-G1T4h.one.H435R |
| hITIP0041 | 187 | c | D | F1_P2insD | FDPM (SEQ ID NO: 371) . . . | hITIP0041-G1T4k.one//VHn-G1T4h.one.H435R |
| hITIP0042 | 188 | c | E | F1_P2insE | FEPM (SEQ ID NO: 372) . . . | hITIP0042-G1T4k.one//VHn-G1T4h.one.H435R |
| hITIP0043 | 189 | c | F | F1_P2insF | FFPM (SEQ ID NO: 373) . . . | hITIP0043-G1T4k.one//VHn-G1T4h.one.H435R |
| hITIP0044 | 190 | c | G | F1_P2insG | FGPM (SEQ ID NO: 374) . . . | hITIP0044-G1T4k.one//VHn-G1T4h.one.H435R |
| hITIP0045 | 191 | c | H | F1_P2insH | FHPM (SEQ ID NO: 375) . . . | hITIP0045-G1T4k.one//VHn-G1T4h.one.H435R |
| hITIP0046 | 192 | c | I | F1_P2insI | FIPM (SEQ ID NO: 376) . . . | hITIP0046-G1T4k.one//VHn-G1T4h.one.H435R |
| hITIP0047 | 193 | c | K | F1_P2insK | FKPM (SEQ ID NO: 377) . . . | hITIP0047-G1T4k.one//VHn-G1T4h.one.H435R |
| hITIP0048 | 194 | c | L | F1_P2insL | FLPM (SEQ ID NO: 378) . . . | hITIP0048-G1T4k.one//VHn-G1T4h.one.H435R |
| hITIP0049 | 195 | c | M | F1_P2insM | FMPM (SEQ ID NO: 379) . . . | hITIP0049-G1T4k.one//VHn-G1T4h.one.H435R |
| hITIP0050 | 196 | c | N | F1_P2insN | FNPM (SEQ ID NO: 380) . . . | hITIP0050-G1T4k.one//VHn-G1T4h.one.H435R |
| hITIP0051 | 197 | c | P | F1_P2insP | FPPM (SEQ ID NO: 381) . . . | hITIP0051-G1T4k.one//VHn-G1T4h.one.H435R |
| hITIP0052 | 198 | c | Q | F1_P2insQ | FQPM (SEQ ID NO: 382) . . . | hITIP0052-G1T4k.one//VHn-G1T4h.one.H435R |
| hlTIPO053 | 199 | c | R | F1_P2insR | FRPM (SEQ ID NO: 383) . . . | hITIP0053-G1T4k.one//VHn-G1T4h.one.H435R |
| hITIP0054 | 200 | c | S | F1_P2insS | FSPM (SEQ ID NO: 384) . . . | hITIP0054-G1T4k.one//VHn-G1T4h.one.H435R |
| hITIP0055 | 201 | c | T | F1_P2insT | FTPM (SEQ ID NO: 385) . . . | hITIP0055-G1T4k.one//VHn-G1T4h.one.H435R |
| hITIP0056 | 202 | c | V | F1_P2insV | FVPM (SEQ ID NO: 386) . . . | hITIP0056-G1T4k.one//VHn-G1T4h.one.H435R |
| hITIP0057 | 203 | c | W | F1_P2insW | FWPM (SEQ ID NO: 387) . . . | hITIP0057-G1T4k.one//VHn-G1T4h.one.H435R |
| hITIP0058 | 204 | c | Y | F1_P2insY | FYPM (SEQ ID NO: 388) . . . | hITIP0058-G1T4k.one//VHn-G1T4h.one.H435R |

### Example 6. Evaluation of the Cell Migration-Inducing Activity of DPPIV-Treated hCXCL10 Variants

Some of the hCXCL10 variants for which cleavage of the two N-terminal residues by hDPPIV was not observed in Example 3 were evaluated as to whether they induce migration of cells expressing CXCR3 (cell migration-inducing activity) even after DPPIV treatment.

Human-derived dipeptidyl peptidase IV (human DPPIV, hDPPIV) (Biolegend, 764102) was used as the protease. The hCXCL10 variants at a final concentration of 12 μM were treated with hDPPIV at a final concentration of 400 nM in PBS under conditions of 37° C. for 1 hour. It was evaluated whether the DPPIV-treated hCXCL10 variants induce migration of cells expressing CXCR3 (cell migration-inducing activity). Cell migration-inducing activity was evaluated using Ba/F3 transfectant cells expressing mouse CXCR3 (mCXCR3) (hereinafter referred to as BaF3/mCXCR3) and HTS Transwell™-96 Permeable Supports with 5.0 μm Pore Polycarbonate Membrane (Cat. 3387, Corning).

The following hCXCL10 variants treated with DPPIV were used as analytes: hCXCL10R75A, hCXCL10R75A.0041, hCXCL10R75A.0042, and hCXCL10R75A.0028.

hCXCL10R75A, hCXCL10R75A.0041, hCXCL10R75A.0042 and hCXCL10R75A.0028 were expressed by transient expression using Expi293 (Life Technologies) by a method known to those skilled in the art, and purification was performed by a method known to those skilled in the art using heparin sepharose (HiTrap Heparin HP Column GE Healthcare) and gel filtration (HiLoadSuperdex75pg GE Healthcare).

After adjusting the final concentration in the solutions of each analyte to be analyzed so as to be from 1 nM to 600 nM, 235 μL of each solution was transferred to the lower chamber. Then, BaF3/mCXCR3 cells were seeded into the upper chamber at 75 μL/well so as to be 2.0×10⁵ cells/well, and the reaction was carried out for 6 hours. The reaction was carried out under the conditions of 5% carbon dioxide and 37° C. After 6 hours of reaction, 100 μL of the solution in the lower chamber was transferred to OptiPlate-96 (Cat. 6005299, PerkinElmer) and 100 μL of CellTiter-Glo™ Luminescent Cell Viability Assay solution (Cat. G7571, Promega) was added. After reacting at room temperature for 10 minutes, the luminescence value was measured with a 2104 EnVision™ multi-label reader (PerkinElmer) to evaluate the level of migration of cells into the lower chamber.

The luminescence intensity reflects the amount of cells that migrated into the lower chamber. The cell migration-inducing activity of hCXCL10 variants was compared.

Comparison of the cell migration-inducing activity of the hCXCL10 variants was performed after 6 hours of reaction, and the results are shown in FIG. 5 and Table 8. DPPIV-treated hCXCL10R75A showed significantly reduced cell migration-inducing activity in the range of 1 nM to 100 nM compared to DPPIV-untreated hCXCL10R75A. On the other hand, DPPIV-treated hCXCL10R75A.0041, hCXCL10R75A.0042 and hCXCL10R75A.0028 showed equivalent cell migration-inducing activity at each concentration as compared with the respective DPPIV-untreated variants at the same concentrations. From this, it was shown that these hCXCL10 variants have sufficient activity even after treatment with DPPIV.

TABLE 8

| | | Analyte | Luminesce |
|---|---|---|---|
| Analyte name | DPPIV | concentration | value |
| hCXCL10R75A | − | 1 nM | 384286 |
| | | 2 nM | 909013 |
| | | 5 nM | 2360051 |
| | | 9 nM | 3925789 |
| | | 19 nM | 5869969 |
| | | 38 nM | 10112159 |
| | | 75 nM | 13262058 |
| | | 150 nM | 9680211 |
| | | 300 nM | 3833908 |
| | | 600 nM | 1126521 |
| hCXCL10R75A.0041 | − | 1 nM | 170183 |
| | | 2 nM | 386200 |
| | | 5 nM | 918247 |
| | | 9 nM | 2393740 |
| | | 19 nM | 4359804 |
| | | 38 nM | 11026260 |
| | | 75 nM | 20180488 |
| | | 150 nM | 19195381 |
| | | 300 nM | 10637776 |
| | | 600 nM | 2996213 |
| hCXCL10R75A.0042 | − | 1 nM | 749550 |
| | | 2 nM | 1053748 |
| | | 5 nM | 1360707 |
| | | 9 nM | 2158386 |
| | | 19 nM | 3573993 |
| | | 38 nM | 10150885 |
| | | 75 nM | 15774662 |
| | | 150 nM | 14178170 |
| | | 300 nM | 8071032 |
| | | 600 nM | 1378453 |
| hCXCL10R75A.0028 | − | 1 nM | 440800 |
| | | 2 nM | 550618 |
| | | 5 nM | 688390 |
| | | 9 nM | 1199380 |
| | | 19 nM | 2142287 |
| | | 38 nM | 7149151 |
| | | 75 nM | 16018027 |
| | | 150 nM | 16362897 |
| | | 300 nM | 12306411 |
| | | 600 nM | 2138988 |
| hCXCL10R75A | + | 1 nM | 78153 |
| | | 2 nM | 73792 |
| | | 5 nM | 74832 |
| | | 9 nM | 84037 |
| | | 19 nM | 82833 |
| | | 38 nM | 107446 |
| | | 75 nM | 410388 |
| | | 150 nM | 1698455 |
| | | 300 nM | 3724658 |
| | | 600 nM | 5453426 |
| hCXCL10R75A.0041 | + | 1 nM | 201203 |
| | | 2 nM | 306204 |
| | | 5 nM | 2158532 |
| | | 9 nM | 3647332 |
| | | 19 nM | 2236899 |
| | | 38 nM | 6118440 |
| | | 75 nM | 20570790 |
| | | 150 nM | 23888168 |
| | | 300 nM | 16714552 |
| | | 600 nM | 3369186 |
| hCXCL10R75A.0042 | + | 1 nM | 984135 |
| | | 2 nM | 1606332 |
| | | 5 nM | 2383033 |
| | | 9 nM | 3822807 |
| | | 19 nM | 5869317 |
| | | 38 nM | 12182978 |
| | | 75 nM | 15343371 |
| | | 150 nM | 11965440 |
| | | 300 nM | 6283371 |
| | | 600 nM | 1624949 |
| hCXCL10R75A.0028 | + | 1 nM | 288701 |
| | | 2 nM | 497223 |
| | | 5 nM | 1222920 |
| | | 9 nM | 2507969 |
| | | 19 nM | 3806319 |
| | | 38 nM | 11819096 |
| | | 75 nM | 16814895 |
| | | 150 nM | 15895458 |
| | | 300 nM | 11558694 |
| | | 600 nM | 3804462 |
| Cell only | − | 0 nM | 99402 |

### Example 7. Evaluation of the Cell Migration-Inducing Activity of hCXCL11 Variant Fc Fusions

It was evaluated whether the hCXCL11 variant Fc fusions prepared in Example 4 induce migration of cells expressing CXCR3 (cell migration-inducing activity). Cell migration-inducing activity was evaluated using Ba/F3 transfectant cells expressing human CXCR3 (hCXCR3) (hereinafter referred to as BaF3/hCXCR3) and HTS Transwell™-96 Permeable Supports with 5.0 μm Pore Polycarbonate Membrane (Cat. 3387, Corning).

The following hCXCL11 variant Fc fusions prepared in Example 4 were used as analytes:
hCXCL11-G1T4k.one//VHn-G1T4h.one.H435R,
hCXCL11.0003-G1T4k.one//VHn-G1T4h.one.H435R,
hCXCL11.0004-G1T4k.one//VHn-G1T4h.one.H435R,
hCXCL11.0005-G1T4k.one//VHn-G1T4h.one.H435R,
hCXCL11.0006-G1T4k.one//VHn-G1T4h.one.H435R,
hCXCL11.0007-G1T4k.one//VHn-G1T4h.one.H435R,
hCXCL11.0008-G1T4k.one//VHn-G1T4h.one.H435R,
hCXCL11.0009-G1T4k.one//VHn-G1T4h.one.H435R,
hCXCL11.0010-G1T4k.one//VHn-G1T4h.one.H435R,
hCXCL11.0011-G1T4k.one//VHn-G1T4h.one.H435R,
hCXCL11.0012-G1T4k.one//VHn-G1T4h.one.H435R,
hCXCL11.0013-G1T4k.one//VHn-G1T4h.one.H435R,
hCXCL11.0014-G1T4k.one//VHn-G1T4h.one.H435R,
hCXCL11.0015-G1T4k.one//VHn-G1T4h.one.H435R,
hCXCL11.0016-G1T4k.one//VHn-G1T4h.one.H435R,
hCXCL11.0017-G1T4k.one//VHn-G1T4h.one.H435R,
hCXCL11.0018-G1T4k.one//VHn-G1T4h.one.H435R,
hCXCL11.0019-G1T4k.one//VHn-G1T4h.one.H435R,
hCXCL11.0020-G1T4k.one//VHn-G1T4h.one.H435R,
hCXCL11.0021-G1T4k.one//VHn-G1T4h.one.H435R,
hCXCL11.0022-G1T4k.one//VHn-G1T4h.one.H435R,
hCXCL11.0023-G1T4k.one//VHn-G1T4h.one.H435R,
hCXCL11.0024-G1T4k.one//VHn-G1T4h.one.H435R,
hCXCL11.0025-G1T4k.one//VHn-G1T4h.one.H435R,
hCXCL11.0026-G1T4k.one//VHn-G1T4h.one.H435R,
hCXCL11.0027-G1T4k.one//VHn-G1T4h.one.H435R,
hCXCL11.0028-G1T4k.one//VHn-G1T4h.one.H435R,
hCXCL11.0029-G1T4k.one//VHn-G1T4h.one.H435R,
hCXCL11.0030-G1T4k.one//VHn-G1T4h.one.H435R,
hCXCL11.0031-G1T4k.one//VHn-G1T4h.one.H435R,
hCXCL11.0032-G1T4k.one//VHn-G1T4h.one.H435R,
hCXCL11.0033-G1T4k.one//VHn-G1T4h.one.H435R,
hCXCL11.0034-G1T4k.one//VHn-G1T4h.one.H435R,
hCXCL11.0035-G1T4k.one//VHn-G1T4h.one.H435R,
hCXCL11.0036-G1T4k.one//VHn-G1T4h.one.H435R,
hCXCL11.0037-G1T4k.one//VHn-G1T4h.one.H435R,
hCXCL11.0038-G1T4k.one//VHn-G1T4h.one.H435R,
hCXCL11.0039-G1T4k.one//VHn-G1T4h.one.H435R,
hCXCL11.0040-G1T4k.one//VHn-G1T4h.one.H435R,
hCXCL11.0041-G1T4k.one//VHn-G1T4h.one.H435R,
hCXCL11.0042-G1T4k.one//VHn-G1T4h.one.H435R,
hCXCL11.0044-G1T4k.one//VHn-G1T4h.one.H435R,
hCXCL11.0045-G1T4k.one//VHn-G1T4h.one.H435R,
hCXCL11.0046-G1T4k.one//VHn-G1T4h.one.H435R,
hCXCL11.0047-G1T4k.one//VHn-G1T4h.one.H435R,
hCXCL11.0048-G1T4k.one//VHn-G1T4h.one.H435R,
hCXCL11.0049-G1T4k.one//VHn-G1T4h.one.H435R,
hCXCL11.0050-G1T4k.one//VHn-G1T4h.one.H435R,
hCXCL11.0051-G1T4k.one//VHn-G1T4h.one.H435R,
hCXCL11.0052-G1T4k.one//VHn-G1T4h.one.H435R,
hCXCL11.0053-G1T4k.one//VHn-G1T4h.one.H435R,
hCXCL11.0054-G1T4k.one//VHn-G1T4h.one.H435R,
hCXCL11.0055-G1T4k.one//VHn-G1T4h.one.H435R,
hCXCL11.0056-G1T4k.one//VHn-G1T4h.one.H435R,
hCXCL11.0057-G1T4k.one//VHn-G1T4h.one.H435R, and
hCXCL11.0058-G1T4k.one//VHn-G1T4h.one.H435R.

After adjusting the final concentration in the solution of each analyte to be analyzed to be 33 nM, 100 nM, and 300 nM, 235 μL of each solution was transferred to the lower chamber. Then, BaF3/hCXCR3 cells were seeded into the upper chamber at 75 μL/well so as to be 2.0×10⁵ cells/well, and the reaction was carried out for 18 hours under the conditions of 5% carbon dioxide and 37° C. After reacting for 18 hours, 100 μL of the solution in the lower chamber was transferred to OptiPlate-96 (Cat. 6005299, PerkinElmer), and 100 μL of CellTiter-Glo™ Luminescent Cell Viability Assay solution (Cat. G7571, Promega) was added. After reacting at room temperature for 10 minutes, the luminescence value was measured with a 2104 EnVision™ multi-label reader (PerkinElmer) to evaluate the level of migration of cells into the lower chamber.

The luminescence intensity reflects the amount of cells that migrated into the lower chamber. The cell migration-inducing activities of hCXCL11-G1T4k.one//VHn-G1T4h.one.H435R and of the other hCXCL11 variant Fc fusions were compared.

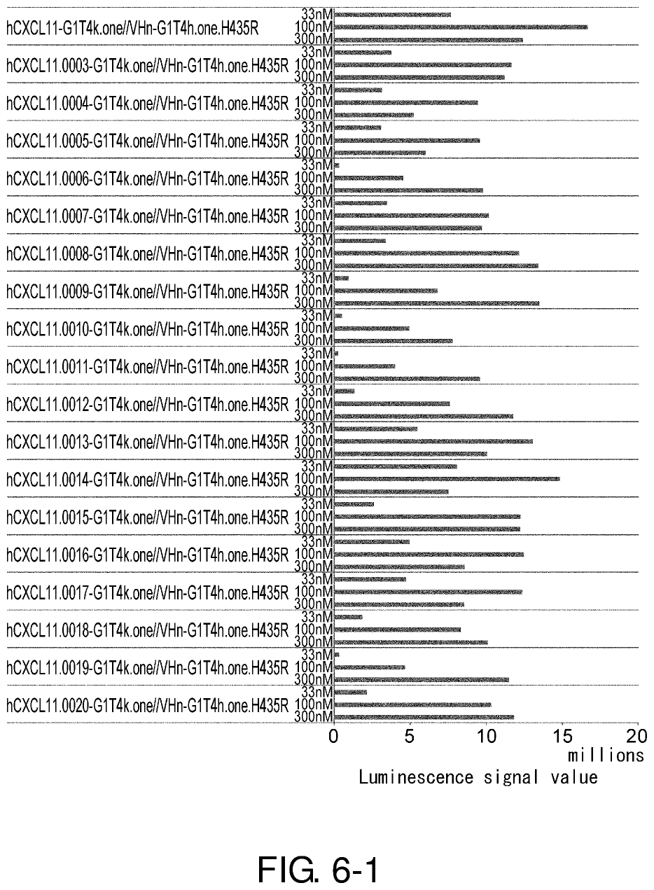

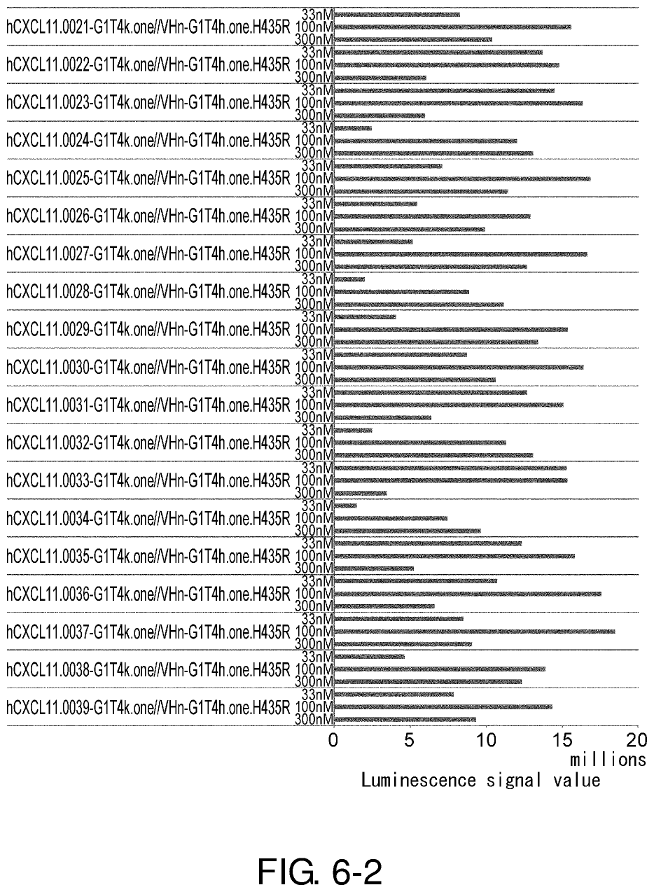

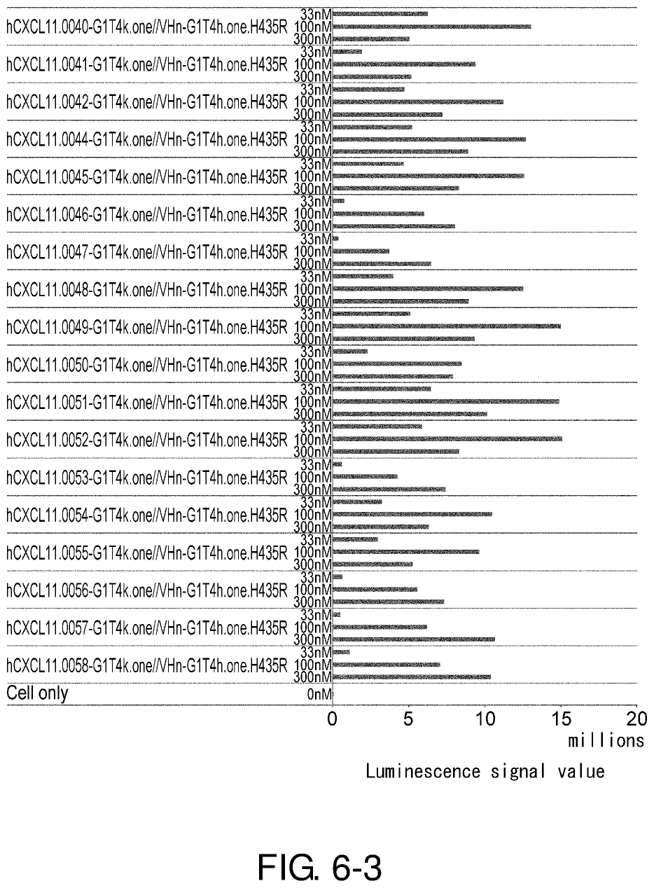

Comparison of the cell migration-inducing activities of hCXCL11-G1T4k.one//VHn-G1T4h.one.H435R and of the other hCXCL11 variant Fc fusions was performed after 18 hours of reaction, and the results are shown in FIG. 6 (FIG. 6-1, FIG. 6-2, and FIG. 6-3) and Table 9. Compared to hCXCL11-G1T4k.one//VHn-G1T4h.one.H435R,
hCXCL11.0003-G1T4k.one//VHn-G1T4h.one.H435R,
hCXCL11.0004-G1T4k.one//VHn-G1T4h.one.H435R,
hCXCL11.0005-G1T4k.one//VHn-G1T4h.one.H435R,
hCXCL11.0007-G1T4k.one//VHn-G1T4h.one.H435R,
hCXCL11.0008-G1T4k.one//VHn-G1T4h.one.H435R,
hCXCL11.0013-G1T4k.one//VHn-G1T4h.one.H435R,
hCXCL11.0014-G1T4k.one//VHn-G1T4h.one.H435R,
hCXCL11.0015-G1T4k.one//VHn-G1T4h.one.H435R,
hCXCL11.0016-G1T4k.one//VHn-G1T4h.one.H435R,
hCXCL11.0017-G1T4k.one//VHn-G1T4h.one.H435R,
hCXCL11.0020-G1T4k.one//VHn-G1T4h.one.H435R,
hCXCL11.0021-G1T4k.one//VHn-G1T4h.one.H435R,
hCXCL11.0022-G1T4k.one//VHn-G1T4h.one.H435R,
hCXCL11.0023-G1T4k.one//VHn-G1T4h.one.H435R,
hCXCL11.0024-G1T4k.one//VHn-G1T4h.one.H435R,
hCXCL11.0025-G1T4k.one//VHn-G1T4h.one.H435R,
hCXCL11.0026-G1T4k.one//VHn-G1T4h.one.H435R,
hCXCL11.0027-G1T4k.one//VHn-G1T4h.one.H435R,
hCXCL11.0028-G1T4k.one//VHn-G1T4h.one.H435R,
hCXCL11.0029-G1T4k.one//VHn-G1T4h.one.H435R,
hCXCL11.0030-G1T4k.one//VHn-G1T4h.one.H435R,
hCXCL11.0031-G1T4k.one//VHn-G1T4h.one.H435R,
hCXCL11.0032-G1T4k.one//VHn-G1T4h.one.H435R,
hCXCL11.0033-G1T4k.one//VHn-G1T4h.one.H435R,
hCXCL11.0035-G1T4k.one//VHn-G1T4h.one.H435R,
hCXCL11.0036-G1T4k.one//VHn-G1T4h.one.H435R,
hCXCL11.0037-G1T4k.one//VHn-G1T4h.one.H435R,
hCXCL11.0038-G1T4k.one//VHn-G1T4h.one.H435R,
hCXCL11.0039-G1T4k.one//VHn-G1T4h.one.H435R,
hCXCL11.0040-G1T4k.one//VHn-G1T4h.one.H435R,
hCXCL11.0041-G1T4k.one//VHn-G1T4h.one.H435R,
hCXCL11.0042-G1T4k.one//VHn-G1T4h.one.H435R,
hCXCL11.0044-G1T4k.one//VHn-G1T4h.one.H435R,
hCXCL11.0045-G1T4k.one//VHn-G1T4h.one.H435R,
hCXCL11.0048-G1T4k.one//VHn-G1T4h.one.H435R,
hCXCL11.0049-G1T4k.one//VHn-G1T4h.one.H435R,
hCXCL11.0050-G1T4k.one//VHn-G1T4h.one.H435R,
hCXCL11.0051-G1T4k.one//VHn-G1T4h.one.H435R,
hCXCL11.0052-G1T4k.one//VHn-G1T4h.one.H435R,
hCXCL11.0054-G1T4k.one//VHn-G1T4h.one.H435R, and
hCXCL11.0055-G1T4k.one//VHn-G1T4h.one.H435R
   retained, at each of the concentrations of 33 nM, 100 nM, and 300 nM, 25% or more of the cell migration-inducing activity of hCXCL11-G1T4k.one//VHn-G1T4h.one.H435R at the same concentration. From this, it was shown that the hCXCL11 variants contained in these hCXCL11 variant Fc fusions have sufficient activity.

TABLE 9

| | Analyte | | Lumines- | Activity |
|---|---|---|---|---|
| | concen- | | cence | retention |
| Analyte name | tration | | value | rate* |
| hCXCL11-G1T4k.one//VHn-G1T4h.one.H435R | 33 | nM | 7680512 | |
| | 100 | nM | 16701834 | |
| | 300 | nM | 12424624 | |
| hCXCL11.0003-G1T4k.one//VHn-G1T4h.one.H435R | 33 | nM | 3761418 | 49% |
| | 100 | nM | 11666034 | 70% |
| | 300 | nM | 11205876 | 90% |
| hCXCL11.0004-G1T4k.one//VHn-G1T4h.one.H435R | 33 | nM | 3145358 | 41% |
| | 100 | nM | 9439905 | 57% |
| | 300 | nM | 5240261 | 42% |
| hCXCL11.0005-G1T4k.one//VHn-G1T4h.one.H435R | 33 | nM | 3107148 | 40% |
| | 100 | nM | 9594341 | 57% |
| | 300 | nM | 6032127 | 49% |
| hCXCL11.0006-G1T4k.one//VHn-G1T4h.one.H435R | 33 | nM | 325725 | 4% |
| | 100 | nM | 4545172 | 27% |
| | 300 | nM | 9793195 | 79% |
| hCXCL11.0007-G1T4k.one//VHn-G1T4h.one.H435R | 33 | nM | 3474336 | 45% |
| | 100 | nM | 10168584 | 61% |
| | 300 | nM | 9705698 | 78% |
| hCXCL11.0008-G1T4k.one//VHn-G1T4h.one.H435R | 33 | nM | 3403401 | 44% |
| | 100 | nM | 12154445 | 73% |
| | 300 | nM | 13423586 | 108% |
| hCXCL11.0009-G1T4k.one//VHn-G1T4h.one.H435R | 33 | nM | 966221 | 13% |
| | 100 | nM | 6792509 | 41% |
| | 300 | nM | 13486638 | 109% |
| hCXCL11.0010-G1T4k.one//VHn-G1T4h.one.H435R | 33 | nM | 524466 | 7% |
| | 100 | nM | 4931525 | 30% |
| | 300 | nM | 7782022 | 63% |
| hCXCL11.0011-G1T4k.one//VHn-G1T4h.one.H435R | 33 | nM | 261903 | 3% |
| | 100 | nM | 4015832 | 24% |
| | 300 | nM | 9604642 | 77% |
| hCXCL11.0012-G1T4k.one//VHn-G1T4h.one.H435R | 33 | nM | 1333862 | 17% |
| | 100 | nM | 7602984 | 46% |
| | 300 | nM | 11766181 | 95% |
| hCXCL11.0013-G1T4k.one//VHn-G1T4h.one.H435R | 33 | nM | 5487357 | 71% |
| | 100 | nM | 13055822 | 78% |
| | 300 | nM | 10050371 | 81% |
| hCXCL11.0014-G1T4k.one//VHn-G1T4h.one.H435R | 33 | nM | 8071684 | 105% |
| | 100 | nM | 14827193 | 89% |
| | 300 | nM | 7512090 | 60% |
| hCXCL11.0015-G1T4k.one//VHn-G1T4h.one.H435R | 33 | nM | 2619355 | 34% |
| | 100 | nM | 12251752 | 73% |
| | 300 | nM | 12226549 | 98% |
| hCXCL11.0016-G1T4k.one//VHn-G1T4h.one.H435R | 33 | nM | 4954461 | 65% |
| | 100 | nM | 12478741 | 75% |
| | 300 | nM | 8570401 | 69% |
| hCXCL11.0017-G1T4k.one//VHn-G1T4h.one.H435R | 33 | nM | 4718379 | 61% |
| | 100 | nM | 12382919 | 74% |
| | 300 | nM | 8542065 | 69% |
| hCXCL11.0018-G1T4k.one//VHn-G1T4h.one.H435R | 33 | nM | 1845181 | 24% |
| | 100 | nM | 8322700 | 50% |
| | 300 | nM | 10085156 | 81% |
| hCXCL11.0019-G1T4k.one//VHn-G1T4h.one.H435R | 33 | nM | 324679 | 4% |
| | 100 | nM | 4663405 | 28% |
| | 300 | nM | 11500114 | 93% |
| hCXCL11.0020-G1T4k.one//VHn-G1T4h.one.H435R | 33 | nM | 2147154 | 28% |
| | 100 | nM | 10335154 | 62% |
| | 300 | nM | 11840783 | 95% |
| hCXCL11.0021-G1T4k.one//VHn-G1T4h.one.H435R | 33 | nM | 8264372 | 108% |
| | 100 | nM | 15582696 | 93% |
| | 300 | nM | 10374927 | 84% |
| hCXCL11.0022-G1T4k.one//VHn-G1T4h.one.H435R | 33 | nM | 13708021 | 178% |
| | 100 | nM | 14788901 | 89% |
| | 300 | nM | 6063758 | 49% |
| hCXCL11.0023-G1T4k.one//VHn-G1T4h.one.H435R | 33 | nM | 14492680 | 189% |
| | 100 | nM | 16351578 | 98% |
| | 300 | nM | 5977170 | 48% |
| hCXCL11.0024-G1T4k.one//VHn-G1T4h.one.H435R | 33 | nM | 2468213 | 32% |
| | 100 | nM | 12017591 | 72% |
| | 300 | nM | 13078459 | 105% |
| hCXCL11.0025-G1T4k.one//VHn-G1T4h.one.H435R | 33 | nM | 7106015 | 93% |
| | 100 | nM | 16854099 | 101% |
| | 300 | nM | 11417885 | 92% |
| hCXCL11.0026-G1T4k.one//VHn-G1T4h.one.H435R | 33 | nM | 5461231 | 71% |
| | 100 | nM | 12896605 | 77% |
| | 300 | nM | 9918162 | 80% |
| hCXCL11.0027-G1T4k.one//VHn-G1T4h.one.H435R | 33 | nM | 5181305 | 67% |
| | 100 | nM | 16650755 | 100% |
| | 300 | nM | 12691313 | 102% |
| hCXCL11.0028-G1T4k.one//VHn-G1T4h.one.H435R | 33 | nM | 1997904 | 26% |
| | 100 | nM | 8871909 | 53% |
| | 300 | nM | 11143349 | 90% |
| hCXCL11.0029-G1T4k.one//VHn-G1T4h.one.H435R | 33 | nM | 4066987 | 53% |
| | 100 | nM | 15360335 | 92% |
| | 300 | nM | 13405229 | 108% |
| hCXCL11.0030-G1T4k.one//VHn-G1T4h.one.H435R | 33 | nM | 8745532 | 114% |
| | 100 | nM | 16405096 | 98% |
| | 300 | nM | 10632773 | 86% |
| hCXCL11.0031-G1T4k.one//VHn-G1T4h.one.H435R | 33 | nM | 12690483 | 165% |
| | 100 | nM | 15076138 | 90% |
| | 300 | nM | 6388637 | 51% |
| hCXCL11.0032-G1T4k.one//VHn-G1T4h.one.H435R | 33 | nM | 2501343 | 33% |
| | 100 | nM | 11321789 | 68% |
| | 300 | nM | 13074388 | 105% |
| hCXCL11.0033-G1T4k.one//VHn-G1T4h.one.H435R | 33 | nM | 15304363 | 199% |
| | 100 | nM | 15347940 | 92% |
| | 300 | nM | 3463231 | 28% |
| hCXCL11.0034-G1T4k.one//VHn-G1T4h.one.H435R | 33 | nM | 1485580 | 19% |
| | 100 | nM | 7452490 | 45% |
| | 300 | nM | 9628060 | 77% |
| hCXCL11.0035-G1T4k.one//VHn-G1T4h.one.H435R | 33 | nM | 12340990 | 161% |
| | 100 | nM | 15832089 | 95% |
| | 300 | nM | 5229763 | 42% |
| hCXCL11.0036-G1T4k.one//VHn-G1T4h.one.H435R | 33 | nM | 10717177 | 140% |
| | 100 | nM | 17569742 | 105% |
| | 300 | nM | 6589358 | 53% |
| hCXCL11.0037-G1T4k.one//VHn-G1T4h.one.H435R | 33 | nM | 8513395 | 111% |
| | 100 | nM | 18473079 | 111% |
| | 300 | nM | 9070191 | 73% |
| hCXCL11.0038-G1T4k.one//VHn-G1T4h.one.H435R | 33 | nM | 4627890 | 60% |
| | 100 | nM | 13910020 | 83% |
| | 300 | nM | 12340351 | 99% |
| hCXCL11.0039-G1T4k.one//VHn-G1T4h.one.H435R | 33 | nM | 7856054 | 102% |
| | 100 | nM | 14332028 | 86% |
| | 300 | nM | 9320232 | 75% |
| hCXCL11.0040-G1T4k.one//VHn-G1T4h.one.H435R | 33 | nM | 6255008 | 81% |
| | 100 | nM | 13071383 | 78% |
| | 300 | nM | 5049092 | 41% |
| hCXCL11.0041-G1T4k.one//VHn-G1T4h.one.H435R | 33 | nM | 1921985 | 25% |
| | 100 | nM | 9373409 | 56% |
| | 300 | nM | 5161844 | 42% |
| hCXCL11.0042-G1T4k.one//VHn-G1T4h.one.H435R | 33 | nM | 4702516 | 61% |
| | 100 | nM | 11224123 | 67% |
| | 300 | nM | 7225056 | 58% |
| hCXCL11.0044-G1T4k.one//VHn-G1T4h.one.H435R | 33 | nM | 5237460 | 68% |
| | 100 | nM | 12713748 | 76% |
| | 300 | nM | 8901072 | 72% |
| hCXCL11.0045-G1T4k.one//VHn-G1T4h.one.H435R | 33 | nM | 4668626 | 61% |
| | 100 | nM | 12595408 | 75% |
| | 300 | nM | 8297150 | 67% |
| hCXCL11.0046-G1T4k.one//VHn-G1T4h.one.H435R | 33 | nM | 760233 | 10% |
| | 100 | nM | 6035270 | 36% |
| | 300 | nM | 8054240 | 65% |
| hCXCL11.0047-G1T4k.one//VHn-G1T4h.one.H435R | 33 | nM | 390951 | 5% |
| | 100 | nM | 3715751 | 22% |
| | 300 | nM | 6480925 | 52% |
| hCXCL11.0048-G1T4k.one//VHn-G1T4h.one.H435R | 33 | nM | 3972971 | 52% |
| | 100 | nM | 12539456 | 75% |
| | 300 | nM | 8954633 | 72% |
| hCXCL11.0049-G1T4k.one//VHn-G1T4h.one.H435R | 33 | nM | 5103080 | 66% |
| | 100 | nM | 15010561 | 90% |
| | 300 | nM | 9323596 | 75% |
| hCXCL11.0050-G1T4k.one//VHn-G1T4h.one.H435R | 33 | nM | 2316728 | 30% |
| | 100 | nM | 8492561 | 51% |
| | 300 | nM | 7921116 | 64% |
| hCXCL11.0051-G1T4k.one//VHn-G1T4h.one.H435R | 33 | nM | 6461187 | 84% |
| | 100 | nM | 14895200 | 89% |
| | 300 | nM | 10154195 | 82% |
| hCXCL11.0052-G1T4k.one//VHn-G1T4h.one.H435R | 33 | nM | 5884538 | 77% |
| | 100 | nM | 15088702 | 90% |
| | 300 | nM | 8326367 | 67% |
| hCXCL11.0053-G1T4k.one//VHn-G1T4h.one.H435R | 33 | nM | 595479 | 8% |
| | 100 | nM | 4263268 | 26% |
| | 300 | nM | 7431787 | 60% |
| hCXCL11.0054-G1T4k.one//VHn-G1T4h.one.H435R | 33 | nM | 3242951 | 42% |
| | 100 | nM | 10496671 | 63% |
| | 300 | nM | 6335806 | 51% |
| hCXCL11.0055-G1T4k.one//VHn-G1T4h.one.H435R | 33 | nM | 2977345 | 39% |
| | 100 | nM | 9651636 | 58% |
| | 300 | nM | 5268238 | 42% |
| hCXCL11.0056-G1T4k.one//VHn-G1T4h.one.H435R | 33 | nM | 643342 | 8% |
| | 100 | nM | 5573043 | 33% |
| | 300 | nM | 7330097 | 59% |
| hCXCL11.0057-G1T4k.one//VHn-G1T4h.one.H435R | 33 | nM | 500643 | 7% |
| | 100 | nM | 6205724 | 37% |
| | 300 | nM | 10672011 | 86% |
| hCXCL11.0058-G1T4k.one//VHn-G1T4h.one.H435R | 33 | nM | 1124561 | 15% |
| | 100 | nM | 7072332 | 42% |
| | 300 | nM | 10398939 | 84% |
| cell only | 0 | nM | 52468 | — |

Activity retention rate*: percentage to the signal value of hCXCL10R75A-G1T4k.one//VHn-G1T4h.one.H435R at the corresponding concentration

### Example 8. Evaluation of the Cell Migration-Inducing Activity of hITIP Variant Fc Fusions

It was evaluated whether the hITIP variant Fc fusions prepared in Example 5 induce migration of cells expressing CXCR3 (cell migration-inducing activity). Cell migration-inducing activity was evaluated using Ba/F3 transfectant cells expressing human CXCR3 (hCXCR3) (hereinafter referred to as BaF3/hCXCR3) and HTS Transwell™-96 Permeable Supports with 5.0 μm Pore Polycarbonate Membrane (Cat. 3387, Corning).

The following hITIP variant Fc fusions prepared in Example 5 were used as analytes:
hITIP-G1T4k.one//VHn-G1T4h.one.H435R,
hITIP0003-G1T4k.one//VHn-G1T4h.one.H435R,
hITIP0004-G1T4k.one//VHn-G1T4h.one.H435R,
hITIP0005-G1T4k.one//VHn-G1T4h.one.H435R,
hITIP0006-G1T4k.one//VHn-G1T4h.one.H435R,
hITIP0007-G1T4k.one//VHn-G1T4h.one.H435R,
hITIP0008-G1T4k.one//VHn-G1T4h.one.H435R,
hITIP0009-G1T4k.one//VHn-G1T4h.one.H435R,
hITIP0010-G1T4k.one//VHn-G1T4h.one.H435R,
hITIP0011-G1T4k.one//VHn-G1T4h.one.H435R,
hITIP0012-G1T4k.one//VHn-G1T4h.one.H435R,
hITIP0013-G1T4k.one//VHn-G1T4h.one.H435R,
hITIP0014-G1T4k.one//VHn-G1T4h.one.H435R,
hITIP0015-G1T4k.one//VHn-G1T4h.one.H435R,
hITIP0016-G1T4k.one//VHn-G1T4h.one.H435R,
hITIP0017-G1T4k.one//VHn-G1T4h.one.H435R,
hITIP0018-G1T4k.one//VHn-G1T4h.one.H435R,
hITIP0019-G1T4k.one//VHn-G1T4h.one.H435R,
hITIP0020-G1T4k.one//VHn-G1T4h.one.H435R,
hITIP0021-G1T4k.one//VHn-G1T4h.one.H435R,
hITIP0022-G1T4k.one//VHn-G1T4h.one.H435R,
hITIP0023-G1T4k.one//VHn-G1T4h.one.H435R,
hITIP0024-G1T4k.one//VHn-G1T4h.one.H435R,
hITIP0025-G1T4k.one//VHn-G1T4h.one.H435R,
hITIP0026-G1T4k.one//VHn-G1T4h.one.H435R,
hITIP0027-G1T4k.one//VHn-G1T4h.one.H435R,
hITIP0028-G1T4k.one//VHn-G1T4h.one.H435R,
hITIP0029-G1T4k.one//VHn-G1T4h.one.H435R,
hITIP0030-G1T4k.one//VHn-G1T4h.one.H435R,
hITIP0031-G1T4k.one//VHn-G1T4h.one.H435R,
hITIP0033-G1T4k.one//VHn-G1T4h.one.H435R,
hITIP0034-G1T4k.one//VHn-G1T4h.one.H435R,
hITIP0035-G1T4k.one//VHn-G1T4h.one.H435R,
hITIP0036-G1T4k.one//VHn-G1T4h.one.H435R,
hITIP0037-G1T4k.one//VHn-G1T4h.one.H435R,
hITIP0038-G1T4k.one//VHn-G1T4h.one.H435R,
hITIP0039-G1T4k.one//VHn-G1T4h.one.H435R,
hITIP0040-G1T4k.one//VHn-G1T4h.one.H435R,
hITIP0041-G1T4k.one//VHn-G1T4h.one.H435R,
hITIP0042-G1T4k.one//VHn-G1T4h.one.H435R,
hITIP0044-G1T4k.one//VHn-G1T4h.one.H435R,
hITIP0045-G1T4k.one//VHn-G1T4h.one.H435R,
hITIP0046-G1T4k.one//VHn-G1T4h.one.H435R,
hITIP0047-G1T4k.one//VHn-G1T4h.one.H435R,
hITIP0048-G1T4k.one//VHn-G1T4h.one.H435R,
hITIP0049-G1T4k.one//VHn-G1T4h.one.H435R,
hITIP0050-G1T4k.one//VHn-G1T4h.one.H435R,
hITIP0051-G1T4k.one//VHn-G1T4h.one.H435R,
hITIP0052-G1T4k.one//VHn-G1T4h.one.H435R,
hITIP0053-G1T4k.one//VHn-G1T4h.one.H435R,
hITIP0054-G1T4k.one//VHn-G1T4h.one.H435R,
hITIP0055-G1T4k.one//VHn-G1T4h.one.H435R,
hITIP0056-G1T4k.one//VHn-G1T4h.one.H435R,
hITIP0057-G1T4k.one//VHn-G1T4h.one.H435R, and
hITIP0058-G1T4k.one//VHn-G1T4h.one.H435R.

After adjusting the final concentration in the solution of each analyte to be analyzed to be 33 nM, 100 nM, and 300 nM, 235 μL of each solution was transferred to the lower chamber. Then, BaF3/hCXCR3 cells were seeded into the upper chamber at 75 L/well so as to be 2.0×10⁵ cells/well, and the reaction was carried out for 18 hours under the conditions of 5% carbon dioxide and 37° C. After reacting for 18 hours, 100 μL of the solution in the lower chamber was transferred to OptiPlate-96 (Cat. 6005299, PerkinElmer), and 100 μL of CellTiter-Glo™ Luminescent Cell Viability Assay solution (Cat. G7571, Promega) was added. After reacting at room temperature for 10 minutes, the luminescence value was measured with a 2104 EnVision™ multi-label reader (PerkinElmer) to evaluate the level of migration of cells into the lower chamber.

The luminescence intensity reflects the amount of cells that migrated into the lower chamber. The cell migration-inducing activities of hITIP-G1T4k.one//VHn-G1T4 h.one.H435R and of the other hITIP variant Fc fusions were compared.

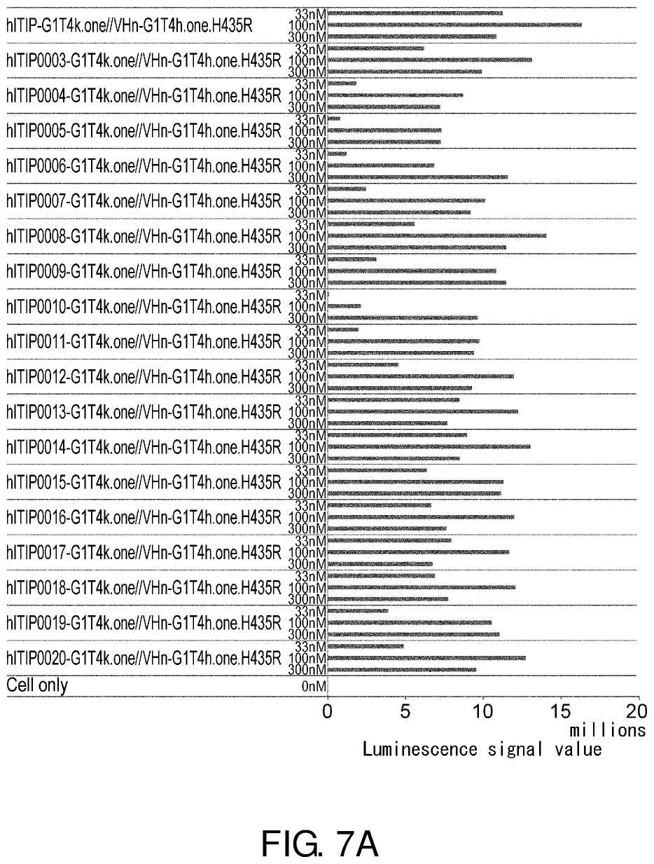

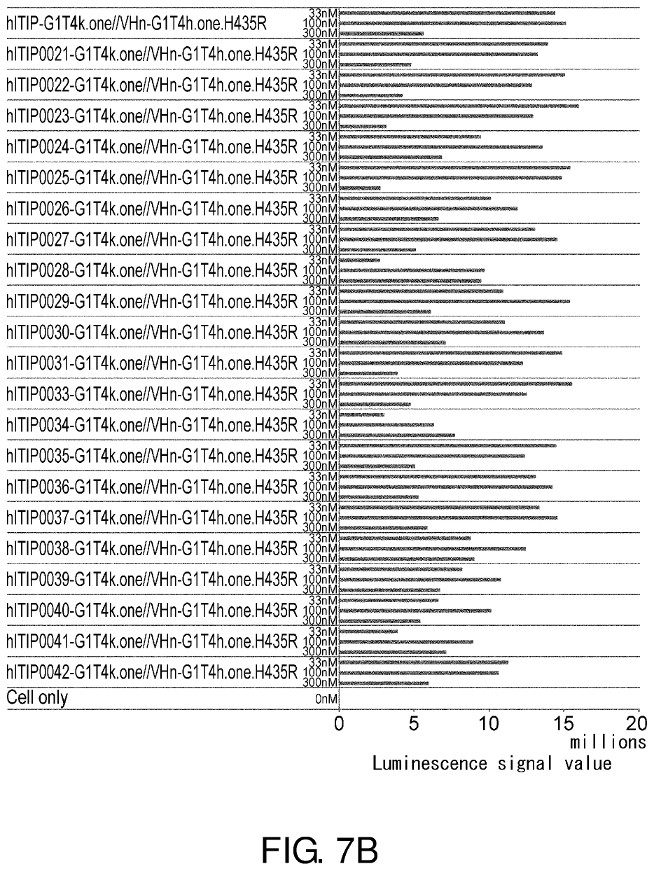

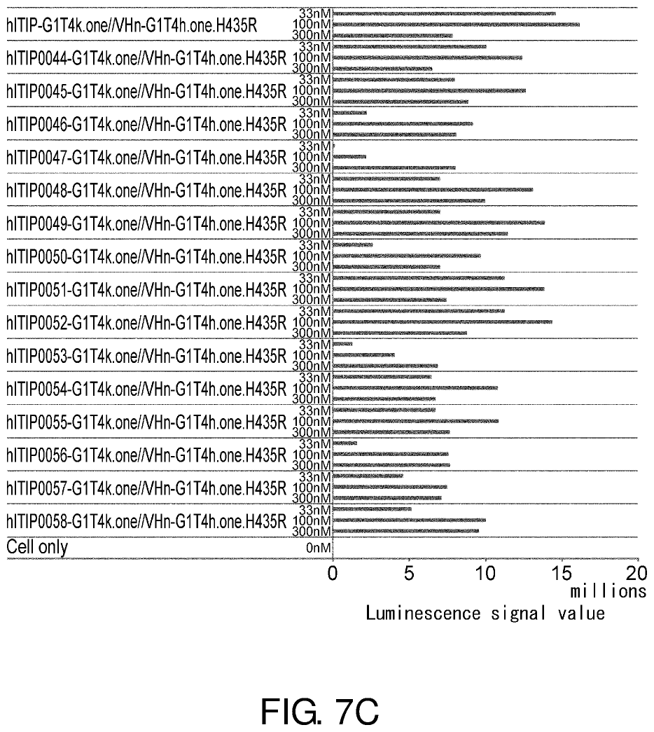

Comparison of the cell migration-inducing activities of hITIP-G1T4k.one//VHn-G1T4h.one.H435R and of the other hITIP variant Fc fusion was performed after 18 hours of reaction, and the results are shown in FIG. 7 (FIG. 7a, FIG. 7b, and FIG. 7c), and Table 10.
hITIP0003-G1T4k.one//VHn-G1T4h.one.H435R,
hITIP0008-G1T4k.one//VHn-G1T4h.one.H435R,
hITIP0009-G1T4k.one//VHn-G1T4h.one.H435R,
hITIP0012-G1T4k.one//VHn-G1T4h.one.H435R,
hITIP0013-G1T4k.one//VHn-G1T4h.one.H435R,
hITIP0014-G1T4k.one//VHn-G1T4h.one.H435R,
hITIP0015-G1T4k.one//VHn-G1T4h.one.H435R,
hITIP0016-G1T4k.one//VHn-G1T4h.one.H435R,
hITIP0017-G1T4k.one//VHn-G1T4h.one.H435R,
hITIP0018-G1T4k.one//VHn-G1T4h.one.H435R,
hITIP0019-G1T4k.one//VHn-G1T4h.one.H435R,
hITIP0020-G1T4k.one//VHn-G1T4h.one.H435R,
hITIP0021-G1T4k.one//VHn-G1T4h.one.H435R,
hITIP0022-G1T4k.one//VHn-G1T4h.one.H435R,
hITIP0023-G1T4k.one//VHn-G1T4h.one.H435R,
hITIP0024-G1T4k.one//VHn-G1T4h.one.H435R,
hITIP0025-G1T4k.one//VHn-G1T4h.one.H435R,
hITIP0026-G1T4k.one//VHn-G1T4h.one.H435R,
hITIP0027-G1T4k.one//VHn-G1T4h.one.H435R,
hITIP0029-G1T4k.one//VHn-G1T4h.one.H435R,
hITIP0030-G1T4k.one//VHn-G1T4h.one.H435R,
hITIP0031-G1T4k.one//VHn-G1T4h.one.H435R,
hITIP0033-G1T4k.one//VHn-G1T4h.one.H435R,
hITIP0035-G1T4k.one//VHn-G1T4h.one.H435R,
hITIP0036-G1T4k.one//VHn-G1T4h.one.H435R,
hITIP0037-G1T4k.one//VHn-G1T4h.one.H435R,
hITIP0038-G1T4k.one//VHn-G1T4h.one.H435R,
hITIP0039-G1T4k.one//VHn-G1T4h.one.H435R,
hITIP0040-G1T4k.one//VHn-G1T4h.one.H435R,
hITIP0041-G1T4k.one//VHn-G1T4h.one.H435R,
hITIP0042-G1T4k.one//VHn-G1T4h.one.H435R,
hITIP0044-G1T4k.one//VHn-G1T4h.one.H435R,
hITIP0045-G1T4k.one//VHn-G1T4h.one.H435R,
hITIP0048-G1T4k.one//VHn-G1T4h.one.H435R,
hITIP0049-G1T4k.one//VHn-G1T4h.one.H435R,
hITIP0051-G1T4k.one//VHn-G1T4h.one.H435R,
hITIP0052-G1T4k.one//VHn-G1T4h.one.H435R,
hITIP0054-G1T4k.one//VHn-G1T4h.one.H435R,
hITIP0055-G1T4k.one//VHn-G1T4h.one.H435R,
hITIP0057-G1T4k.one//VHn-G1T4h.one.H435R, and
hITIP0058-G1T4k.one//VHn-G1T4h.one.H435R
retained, at each of the concentration of 33 nM, 100 nM, 300 nM, 25% or more of cell migration-inducing activity of hITIP-G1T4k.one//VHn-G1T4h.one.H435R at the same concentration. From this, it was shown that the hITIP variants contained in these hITIP variant Fc fusions have sufficient activity.

TABLE 10

| | | | |
|---|---|---|---|
| | Analyte | Lumines- | Activity |
| | concen- | cence | retention |
| Analyte name | tration | value | rate* |

Sample set_1

| | | | | |
|---|---|---|---|---|
| hITIP-G1T4k.one//VHn-G1T4h.one.H435R | 33 | nM | 11255810 | |
| | 100 | nM | 16333190 | |
| | 300 | nM | 10853981 | |
| hITIP0003-G1T4k.one//VHn-G1T4h.one.H435R | 33 | nM | 6184897 | 55% |
| | 100 | nM | 13130438 | 80% |
| | 300 | nM | 9909618 | 91% |
| hITIP0004-G1T4k.one//VHn-G1T4h.one.H435R | 33 | nM | 1864821 | 17% |
| | 100 | nM | 8714209 | 53% |
| | 300 | nM | 7226926 | 67% |
| hITIP0005-G1T4k.one//VHn-G1T4h.one.H435R | 33 | nM | 802531 | 7% |
| | 100 | nM | 7331671 | 45% |
| | 300 | nM | 7271155 | 67% |
| hITIP0006-G1T4k.one//VHn-G1T4h.one.H435R | 33 | nM | 1230226 | 11% |
| | 100 | nM | 6851471 | 42% |
| | 300 | nM | 11591358 | 107% |
| hITIP0007-G1T4k.one//VHn-G1T4h.one.H435R | 33 | nM | 2479379 | 22% |
| | 100 | nM | 10108277 | 62% |
| | 300 | nM | 9182812 | 85% |
| hITIP0008-G1T4k.one//VHn-G1T4h.one.H435R | 33 | nM | 5585908 | 50% |
| | 100 | nM | 14059020 | 86% |
| | 300 | nM | 11481733 | 106% |
| hITIP0009-G1T4k.one//VHn-G1T4h.one.H435R | 33 | nM | 3141421 | 28% |
| | 100 | nM | 10831129 | 66% |
| | 300 | nM | 11479957 | 106% |
| hITIP0010-G1T4k.one//VHn-G1T4h.one.H435R | 33 | nM | 104208 | 1% |
| | 100 | nM | 2139235 | 13% |
| | 300 | nM | 9641767 | 89% |
| hITIP0011-G1T4k.one//VHn-G1T4h.one.H435R | 33 | nM | 2012679 | 18% |
| | 100 | nM | 9741847 | 60% |
| | 300 | nM | 9395780 | 87% |
| hITIP0012-G1T4k.one//VHn-G1T4h.one.H435R | 33 | nM | 4553615 | 40% |
| | 100 | nM | 11974754 | 73% |
| | 300 | nM | 9283034 | 86% |
| hITIP0013-G1T4k.one//VHn-G1T4h.one.H435R | 33 | nM | 8489369 | 75% |
| | 100 | nM | 12237110 | 75% |
| | 300 | nM | 7711718 | 71% |
| hITIP0014-G1T4k.one//VHn-G1T4h.one.H435R | 33 | nM | 8972343 | 80% |
| | 100 | nM | 13028436 | 80% |
| | 300 | nM | 8481716 | 78% |
| hITIP0015-G1T4k.one//VHn-G1T4h.one.H435R | 33 | nM | 6378932 | 57% |
| | 100 | nM | 11292522 | 69% |
| | 300 | nM | 11163432 | 103% |
| hITIP0016-G1T4k.one//VHn-G1T4h.one.H435R | 33 | nM | 6656446 | 59% |
| | 100 | nM | 11995226 | 73% |
| | 300 | nM | 7646801 | 70% |
| hITIP0017-G1T4k.one//VHn-G1T4h.one.H435R | 33 | nM | 7957520 | 71% |
| | 100 | nM | 11671324 | 71% |
| | 300 | nM | 6762275 | 62% |
| hITIP0018-G1T4k.one//VHn-G1T4h.one.H435R | 33 | nM | 6897647 | 61% |
| | 100 | nM | 12083915 | 74% |
| | 300 | nM | 7743185 | 71% |
| hIlTIP0019-G1T4k.one//VHn-G1T4h.one.H435R | 33 | nM | 3893156 | 35% |
| | 100 | nM | 10537283 | 65% |
| | 300 | nM | 11047471 | 102% |
| hITIP0020-G1T4k.one//VHn-G1T4h.one.H435R | 33 | nM | 4898429 | 44% |
| | 100 | nM | 12726742 | 78% |
| | 300 | nM | 9561501 | 88% |

Sample set_2

| | | | | |
|---|---|---|---|---|
| hITIP-G1T4k.one//VHn-G1T4h.one.H435R | 33 | nM | 14416819 | |
| | 100 | nM | 15131844 | |
| | 300 | nM | 5642551 | |
| hITIP0021-G1T4k.one//VHn-G1T4h.one.H435R | 33 | nM | 13956263 | 97% |
| | 100 | nM | 13254701 | 88% |
| | 300 | nM | 4839338 | 86% |
| hITIP0022-G1T4k.one//VHn-G1T4h.one.H435R | 33 | nM | 15061363 | 104% |
| | 100 | nM | 12879459 | 85% |
| | 300 | nM | 4246847 | 75% |
| hITIP0023-G1T4k.one//VHn-G1T4h.one.H435R | 33 | nM | 15979606 | 111% |
| | 100 | nM | 12935580 | 85% |
| | 300 | nM | 3156988 | 56% |
| hITIP0024-G1T4k.one//VHn-G1T4h.one.H435R | 33 | nM | 9470253 | 66% |
| | 100 | nM | 13585196 | 90% |
| | 300 | nM | 6872305 | 122% |
| hITIP0025-G1T4k.one//VHn-G1T4h.one.H435R | 33 | nM | 15422760 | 107% |
| | 100 | nM | 14866297 | 98% |
| | 300 | nM | 2767805 | 49% |
| hITIP0026-G1T4k.one//VHn-G1T4h.one.H435R | 33 | nM | 10128027 | 70% |
| | 100 | nM | 11888103 | 79% |
| | 300 | nM | 6631111 | 118% |
| hITIP0027-G1T4k.one//VHn-G1T4h.one.H435R | 33 | nM | 13086406 | 91% |
| | 100 | nM | 14555849 | 96% |
| | 300 | nM | 5126848 | 91% |
| hITIP0028-G1T4k.one//VHn-G1T4h.one.H435R | 33 | nM | 2744330 | 19% |
| | 100 | nM | 9691581 | 64% |
| | 300 | nM | 9498848 | 168% |
| hITIP0029-G1T4k.one//VHn-G1T4h.one.H435R | 33 | nM | 10941109 | 76% |
| | 100 | nM | 15408714 | 102% |
| | 300 | nM | 6128140 | 109% |
| hITIP0030-G1T4k.one//VHn-G1T4h.one.H435R | 33 | nM | 11055133 | 77% |
| | 100 | nM | 13666700 | 90% |
| | 300 | nM | 7108276 | 126% |
| hITIP0031-G1T4k.one//VHn-G1T4h.one.H435R | 33 | nM | 14905473 | 103% |
| | 100 | nM | 12274657 | 81% |
| | 300 | nM | 3921392 | 69% |
| hITIP0033-G1T4k.one//VHn-G1T4h.one.H435R | 33 | nM | 15539594 | 108% |
| | 100 | nM | 12523151 | 83% |
| | 300 | nM | 4770826 | 85% |
| hITIP0034-G1T4k.one//VHn-G1T4h.one.H435R | 33 | nM | 3014026 | 21% |
| | 100 | nM | 6337835 | 42% |
| | 300 | nM | 7730336 | 137% |
| hITIP0035-G1T4k.one//VHn-G1T4h.one.H435R | 33 | nM | 14479491 | 100% |
| | 100 | nM | 12391946 | 82% |
| | 300 | nM | 5087719 | 90% |
| hITIP0036-G1T4k.one//VHn-G1T4h.one.H435R | 33 | nM | 13125449 | 91% |
| | 100 | nM | 14230048 | 94% |
| | 300 | nM | 5303773 | 94% |
| hITIP0037-G1T4k.one//VHn-G1T4h.one.H435R | 33 | nM | 13362538 | 93% |
| | 100 | nM | 14557898 | 96% |
| | 300 | nM | 5896904 | 105% |
| hITIP0038-G1T4k.one//VHn-G1T4h.one.H435R | 33 | nM | 8778686 | 61% |
| | 100 | nM | 12447834 | 82% |
| | 300 | nM | 9021217 | 160% |
| hITIP0039-G1T4k.one//VHn-G1T4h.one.H435R | 33 | nM | 8218579 | 57% |
| | 100 | nM | 10785484 | 71% |
| | 300 | nM | 6753122 | 120% |
| hITIP0040-G1T4k.one//VHn-G1T4h.one.H435R | 33 | nM | 6641961 | 46% |
| | 100 | nM | 10152246 | 67% |
| | 300 | nM | 5422998 | 96% |
| hITIP0041-G1T4k.one//VHn-G1T4h.one.H435R | 33 | nM | 3911869 | 27% |
| | 100 | nM | 8945206 | 59% |
| | 300 | nM | 7154135 | 127% |
| hITIP0042-G1T4k.one//VHn-G1T4h.one.H435R | 33 | nM | 11287798 | 78% |
| | 100 | nM | 10640127 | 70% |
| | 300 | nM | 5991955 | 106% |

Sample set_3

| | | | | |
|---|---|---|---|---|
| hITIP-G1T4k.one//VHn-G1T4h.one.H435R | 33 | nM | 14582717 | |
| | 100 | nM | 16151041 | |
| | 300 | nM | 7844794 | |
| hITIP0044-G1T4k.one//VHn-G1T4h.one.H435R | 33 | nM | 10045938 | 69% |
| | 100 | nM | 12391082 | 77% |
| | 300 | nM | 6527919 | 83% |
| hITIP0045-G1T4k.one//VHn-G1T4h.one.H435R | 33 | nM | 7976121 | 55% |
| | 100 | nM | 12629509 | 78% |
| | 300 | nM | 8864666 | 113% |
| hITIP0046-G1T4k.one//VHn-G1T4h.one.H435R | 33 | nM | 2230683 | 15% |
| | 100 | nM | 9165961 | 57% |
| | 300 | nM | 8091881 | 103% |
| hITIP0047-G1T4k.one//VHn-G1T4h.one.H435R | 33 | nM | 158870 | 1% |
| | 100 | nM | 2192199 | 14% |
| | 300 | nM | 8023795 | 102% |
| hITIP0048-G1T4k.one//VHn-G1T4h.one.H435R | 33 | nM | 7012064 | 48% |
| | 100 | nM | 13095826 | 81% |
| | 300 | nM | 9972870 | 127% |
| hITIP0049-G1T4k.one//VHn-G1T4h.one.H435R | 33 | nM | 7019689 | 48% |
| | 100 | nM | 13895669 | 86% |
| | 300 | nM | 11446853 | 146% |
| hITIP0050-G1T4k.one//VHn-G1T4h.one.H435R | 33 | nM | 2605315 | 18% |
| | 100 | nM | 9676705 | 60% |
| | 300 | nM | 7018480 | 89% |
| hITIP0051-G1T4k.one//VHn-G1T4h.one.H435R | 33 | nM | 11235454 | 77% |
| | 100 | nM | 13839262 | 86% |
| | 300 | nM | 7446291 | 95% |
| hITIP0052-G1T4k.one//VHn-G1T4h.one.H435R | 33 | nM | 11235767 | 77% |
| | 100 | nM | 14357609 | 89% |
| | 300 | nM | 8783137 | 112% |
| hITIP0053-G1T4k.one//VHn-G1T4h.one.H435R | 33 | nM | 1289632 | 9% |
| | 100 | nM | 4047257 | 25% |
| | 300 | nM | 6884729 | 88% |
| hITIP0054-G1T4k.one//VHn-G1T4h.one.H435R | 33 | nM | 6461440 | 44% |
| | 100 | nM | 10787916 | 67% |
| | 300 | nM | 6726740 | 86% |
| hITIP0055-G1T4k.one//VHn-G1T4h.one.H435R | 33 | nM | 6716736 | 46% |
| | 100 | nM | 10851741 | 67% |
| | 300 | nM | 7652397 | 98% |
| hITIP0056-G1T4k.one//VHn-G1T4h.one.H435R | 33 | nM | 1586824 | 11% |
| | 100 | nM | 7566720 | 47% |
| | 300 | nM | 7679419 | 98% |
| hITIP0057-G1T4k.one//VHn-G1T4h.one.H435R | 33 | nM | 4605304 | 32% |
| | 100 | nM | 7485005 | 46% |
| | 300 | nM | 7123475 | 91% |
| hITIP0058-G1T4k.one//VHn-G1T4h.one.H435R | 33 | nM | 5163373 | 35% |
| | 100 | nM | 10015503 | 62% |
| | 300 | nM | 9561334 | 122% |
| cell only | 0 | nM | 31290 | — |

Activity retention rate*: percentage to the signal value of hITIP-G1T4k.one//VHn-G1T4h.one.H435R at the corresponding concentration

### Example 9. Evaluation of DPPIV Cleavage Resistance of hCXCL11 Variant Fc Fusions and hITIP Variant Fc Fusions

It was examined whether the hCXCL11 variant Fc fusions and the hITIP variant Fc fusions that induced cell migration in Examples 7 and 8 are cleaved by dipeptidyl peptidase IV (DPPIV). Human DPPIV (hDPPIV) (Biolegend, 764102) derived from human was used as DPPIV. Cleavage of hCXCL11 variant Fc fusions and hITIP variant Fc fusions by DPPIV was evaluated by LC/MS analysis after N-type deglycosylation and reduction treatment. After reacting hCXCL11 variant Fc fusions and hITIP variant Fc fusions at a final concentration of 0.2 mg/ml (3.3 μM) and hDPPIV at a final concentration of 200 nM in PBS for 1 hour at 37° C., LC/MS analysis was performed by a method known to those skilled in the art, and the cleavage of the hCXCL11 variant Fc fusions and hITIP variant Fc fusions was evaluated.

For hCXCL11-G1T4k.one//VHn-G1T4h.one.H435R and hITIP-G1T4k.one//VHn-G1T4h.one.H435R, DPPIV treatment reduced each of the masses of the hCXCL11 variant-containing peptide chain and the hITIP variant-containing peptide chain. The masses corresponded to the theoretical masses of the sequences of each of the hCXCL11 variant-containing peptide chain and hITIP variant-containing peptide chain lacking the two amino acid residues from the N-terminal. This suggested that the two amino acid residues from the N-terminal of hCXCL11 and hITIP contained in hCXCL11-G1T4k.one//VHn-G1T4h.one.H435R and hITIP-G1T4k.one//VHn-G1T4h.one.H435R were cleaved by hDPPIV.

On the other hand,
hCXCL11.0021-G1T4k.one//VHn-G1T4h.one.H435R
hCXCL11.0022-G1T4k.one//VHn-G1T4h.one.H435R,
hCXCL11.0023-G1T4k.one//VHn-G1T4h.one.H435R,
hCXCL11.0025-G1T4k.one//VHn-G1T4h.one.H435R,
hCXCL11.0030-G1T4k.one//VHn-G1T4h.one.H435R,
hCXCL11.0031-G1T4k.one//VHn-G1T4h.one.H435R,
hCXCL11.0033-G1T4k.one//VHn-G1T4h.one.H435R,
hCXCL11.0035-G1T4k.one//VHn-G1T4h.one.H435R,
hCXCL11.0036-G1T4k.one//VHn-G1T4h.one.H435R,
hCXCL11.0037-G1T4k.one//VHn-G1T4h.one.H435R,
hCXCL11.0039-G1T4k.one//VHn-G1T4h.one.H435R,
hITIP0021-G1T4k.one//VHn-G1T4h.one.H435R,
hITIP0022-G1T4k.one//VHn-G1T4h.one.H435R,
hITIP0023-G1T4k.one//VHn-G1T4h.one.H435R,
hITIP0025-G1T4k.one//VHn-G1T4h.one.H435R,
hITIP0030-G1T4k.one//VHn-G1T4h.one.H435R,
hITIP0031-G1T4k.one//VHn-G1T4h.one.H435R,
hITIP0033-G1T4k.one//VHn-G1T4h.one.H435R,
hITIP0035-G1T4k.one//VHn-G1T4h.one.H435R,
hITIP0036-G1T4k.one//VHn-G1T4h.one.H435R,
hITIP0037-G1T4k.one//VHn-G1T4h.one.H435R, and
hITIP0039-G1T4k.one//VHn-G1T4h.one.H435R
   showed no mass change suggestive of peptide bond cleavage by hDPPIV treatment. From this result, it was suggested that the hCXCL11 variants and hITIP contained in these hCXCL11 variant Fc fusions and hITIP variant Fc fusions show hDPPIV cleavage resistance.

Since the two amino acid residues from the N-terminal of hCXCL11 and hITIP are cleaved by hDPPIV, it was suggested that the amino acid modifications in and around the DPPIV recognition/cleavage site contained in the above hCXCL11 variants and hITIP variants that did not show a mass change due to hDPPIV treatment are useful modifications to provide DPPIV resistance to hCXCL11 and hITIP.

Although the foregoing invention has been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

### INDUSTRIAL APPLICABILITY

The present disclosure is the first disclosure of CXCR3 ligands having resistance to DPPIV and also having the activity to cause migration of cells expressing CXCR3. Such CXCR3 ligands are useful for disease treatment/prevention by causing migration of cells expressing CXCR3.

1. A method of producing a CXCR3 ligand having resistance to DPPIV, wherein the method comprises modifying a parent CXCR3 ligand in which the 2^{nd} amino acid from the N-terminus is P or A, wherein the method comprises at least one of the following:
(1) substituting the 2^{nd} amino acid from the N-terminus of the parent CXCR3 ligand from P or A to F, G, I, K, L, M, T, V, W, or Y;
(2) further adding A, F, G, H, I, K, L, M, N, P, Q, R, S, T, V, W, or Y to the N-terminus of the parent CXCR3 ligand;
(3) inserting A, F, G, H, I, K, L, M, P, Q, R, S, T, V, W, or Y between the 1^{st} and 2^{nd} amino acids at the N-terminus of the parent CXCR3 ligand; and
(4) deleting the V at the N-terminus of the parent CXCR3 ligand.

2. The method according to claim 1, wherein the parent CXCR3 ligand further has a C-X-C motif.

3. The method according to claim 2, wherein the C-X-C motif is C-T-C (Cys-Thr-Cys), C-L-C (Cys-Leu-Cys), or C-S-C (Cys-Ser-Cys).

4. The method according to claim 1, wherein the parent CXCR3 ligand is either a naturally-occurring human CXCL10 or a variant thereof, a naturally-occurring human CXCL11 or a variant thereof, or a naturally-occurring human CXCL9 or a variant thereof.

5. The method according to claim 1, wherein the 1^{st} amino acid at the N-terminus of the parent CXCR3 ligand is V.

6. The method according to claim 1, wherein the N-terminal sequence of the parent CXCR3 ligand is VPLSRTVR (SEQ ID NO: 86) or VALSRTVR (SEQ ID NO: 87).

7. The method according to claim 6, wherein the parent C-X-C motif is located at the C-terminus of the N-terminal sequence “V-P-L-S-R-T-V-R (SEQ ID NO: 86)” or “V-A-L-S-R-T-V-R (SEQ ID NO: 87)”.

8. A method of producing a CXCR3 ligand having resistance to DPPIV, wherein the method comprises modifying a parent CXCR3 ligand in which the 2^{nd} amino acid from the N-terminus is P, wherein the method comprises at least one of the following:
(1) further adding A, D, E, F, G, H, I, K, L, M, N, P, Q, S, T, V, W, or Y to the N-terminus of the parent CXCR3 ligand;
(2) substituting the 2^{nd} amino acid from the N-terminus of the parent CXCR3 ligand from P to A, D, E, G, H, I, M, N, Q, R, S, T, V, W, or Y; and
(3) inserting A, D, E, F, G, H, L, M, N, P, Q, S, T, W, or Y between the 1^{st} and 2^{nd} amino acids at the N-terminus of the parent CXCR3 ligand.

9. The method according to claim 8, wherein the parent CXCR3 ligand further has a C-X-C motif.

10. The method according to claim 9, wherein the C-X-C motif is C-T-C (Cys-Thr-Cys), C-L-C (Cys-Leu-Cys), or C-S-C (Cys-Ser-Cys).

11. The method according to claim 8, wherein the parent CXCR3 ligand is either a naturally-occurring human CXCL10 or a variant thereof, a naturally-occurring human CXCL11 or a variant thereof, or a naturally-occurring human CXCL9 or a variant thereof.

12. The method according to claim 8, wherein the 1^{st} amino acid at the N-terminus of the parent CXCR3 ligand is F.

13. The method according to claim 8, wherein the N-terminal sequence of the parent CXCR3 ligand is FPMFKRGR (SEQ ID NO: 91).

14. The method according to claim 13, wherein the parent C-X-C motif is located at the C-terminus of the N-terminal sequence “F-P-M-F-K-R-G-R (SEQ ID NO: 91)”.


SEQUENCE LISTING (388 sequences)

SEQ ID NO: 1 - protein, 77 aa, synthetic construct
  REGION 1..77: note = an artificially synthesized sequence
VPLSRTVRCTCISISNQPVNPRSLEKLEIIPASQFCPRVEIIATMKKKGEKRCLNPESKAIKNLLKAVSKERSKASP

SEQ ID NO: 2 - protein, 77 aa, synthetic construct
  REGION 1..77: note = an artificially synthesized sequence
VALSRTVRCTCISISNQPVNPRSLEKLEIIPASQFCPRVEIIATMKKKGEKRCLNPESKAIKNLLKAVSKERSKASP

SEQ ID NO: 3 - protein, 77 aa, synthetic construct
  REGION 1..77: note = an artificially synthesized sequence
VDLSRTVRCTCISISNQPVNPRSLEKLEIIPASQFCPRVEIIATMKKKGEKRCLNPESKAIKNLLKAVSKERSKASP

SEQ ID NO: 4 - protein, 77 aa, synthetic construct
  REGION 1..77: note = an artificially synthesized sequence
VELSRTVRCTCISISNQPVNPRSLEKLEIIPASQFCPRVEIIATMKKKGEKRCLNPESKAIKNLLKAVSKERSKASP

SEQ ID NO: 5 - protein, 77 aa, synthetic construct
  REGION 1..77: note = an artificially synthesized sequence
VFLSRTVRCTCISISNQPVNPRSLEKLEIIPASQFCPRVEIIATMKKKGEKRCLNPESKAIKNLLKAVSKERSKASP

SEQ ID NO: 6 - protein, 77 aa, synthetic construct
  REGION 1..77: note = an artificially synthesized sequence
VGLSRTVRCTCISISNQPVNPRSLEKLEIIPASQFCPRVEIIATMKKKGEKRCLNPESKAIKNLLKAVSKERSKASP

SEQ ID NO: 7 - protein, 77 aa, synthetic construct
  REGION 1..77: note = an artificially synthesized sequence
VHLSRTVRCTCISISNQPVNPRSLEKLEIIPASQFCPRVEIIATMKKKGEKRCLNPESKAIKNLLKAVSKERSKASP

SEQ ID NO: 8 - protein, 77 aa, synthetic construct
  REGION 1..77: note = an artificially synthesized sequence
VILSRTVRCTCISISNQPVNPRSLEKLEIIPASQFCPRVEIIATMKKKGEKRCLNPESKAIKNLLKAVSKERSKASP

SEQ ID NO: 9 - protein, 77 aa, synthetic construct
  REGION 1..77: note = an artificially synthesized sequence
VKLSRTVRCTCISISNQPVNPRSLEKLEIIPASQFCPRVEIIATMKKKGEKRCLNPESKAIKNLLKAVSKERSKASP

SEQ ID NO: 10 - protein, 77 aa, synthetic construct
  REGION 1..77: note = an artificially synthesized sequence
VLLSRTVRCTCISISNQPVNPRSLEKLEIIPASQFCPRVEIIATMKKKGEKRCLNPESKAIKNLLKAVSKERSKASP

SEQ ID NO: 11 - protein, 77 aa, synthetic construct
  REGION 1..77: note = an artificially synthesized sequence
VMLSRTVRCTCISISNQPVNPRSLEKLEIIPASQFCPRVEIIATMKKKGEKRCLNPESKAIKNLLKAVSKERSKASP

SEQ ID NO: 12 - protein, 77 aa, synthetic construct
  REGION 1..77: note = an artificially synthesized sequence
VNLSRTVRCTCISISNQPVNPRSLEKLEIIPASQFCPRVEIIATMKKKGEKRCLNPESKAIKNLLKAVSKERSKASP

SEQ ID NO: 13 - protein, 77 aa, synthetic construct
  REGION 1..77: note = an artificially synthesized sequence
VQLSRTVRCTCISISNQPVNPRSLEKLEIIPASQFCPRVEIIATMKKKGEKRCLNPESKAIKNLLKAVSKERSKASP

SEQ ID NO: 14 - protein, 77 aa, synthetic construct
  REGION 1..77: note = an artificially synthesized sequence
VRLSRTVRCTCISISNQPVNPRSLEKLEIIPASQFCPRVEIIATMKKKGEKRCLNPESKAIKNLLKAVSKERSKASP

SEQ ID NO: 15 - protein, 77 aa, synthetic construct
  REGION 1..77: note = an artificially synthesized sequence
VSLSRTVRCTCISISNQPVNPRSLEKLEIIPASQFCPRVEIIATMKKKGEKRCLNPESKAIKNLLKAVSKERSKASP

SEQ ID NO: 16 - protein, 77 aa, synthetic construct
  REGION 1..77: note = an artificially synthesized sequence
VTLSRTVRCTCISISNQPVNPRSLEKLEIIPASQFCPRVEIIATMKKKGEKRCLNPESKAIKNLLKAVSKERSKASP

SEQ ID NO: 17 - protein, 77 aa, synthetic construct
  REGION 1..77: note = an artificially synthesized sequence
VVLSRTVRCTCISISNQPVNPRSLEKLEIIPASQFCPRVEIIATMKKKGEKRCLNPESKAIKNLLKAVSKERSKASP

SEQ ID NO: 18 - protein, 77 aa, synthetic construct
  REGION 1..77: note = an artificially synthesized sequence
VWLSRTVRCTCISISNQPVNPRSLEKLEIIPASQFCPRVEIIATMKKKGEKRCLNPESKAIKNLLKAVSKERSKASP

SEQ ID NO: 19 - protein, 77 aa, synthetic construct
  REGION 1..77: note = an artificially synthesized sequence
VYLSRTVRCTCISISNQPVNPRSLEKLEIIPASQFCPRVEIIATMKKKGEKRCLNPESKAIKNLLKAVSKERSKASP

SEQ ID NO: 20 - protein, 78 aa, synthetic construct
  REGION 1..78: note = an artificially synthesized sequence
AVPLSRTVRCTCISISNQPVNPRSLEKLEIIPASQFCPRVEIIATMKKKGEKRCLNPESKAIKNLLKAVSKERSKASP

SEQ ID NO: 21 - protein, 78 aa, synthetic construct
  REGION 1..78: note = an artificially synthesized sequence
DVPLSRTVRCTCISISNQPVNPRSLEKLEIIPASQFCPRVEIIATMKKKGEKRCLNPESKAIKNLLKAVSKERSKASP

SEQ ID NO: 22 - protein, 78 aa, synthetic construct
  REGION 1..78: note = an artificially synthesized sequence
EVPLSRTVRCTCISISNQPVNPRSLEKLEIIPASQFCPRVEIIATMKKKGEKRCLNPESKAIKNLLKAVSKERSKASP

SEQ ID NO: 23 - protein, 78 aa, synthetic construct
  REGION 1..78: note = an artificially synthesized sequence
FVPLSRTVRCTCISISNQPVNPRSLEKLEIIPASQFCPRVEIIATMKKKGEKRCLNPESKAIKNLLKAVSKERSKASP

SEQ ID NO: 24 - protein, 78 aa, synthetic construct
  REGION 1..78: note = an artificially synthesized sequence
GVPLSRTVRCTCISISNQPVNPRSLEKLEIIPASQFCPRVEIIATMKKKGEKRCLNPESKAIKNLLKAVSKERSKASP

SEQ ID NO: 25 - protein, 78 aa, synthetic construct
  REGION 1..78: note = an artificially synthesized sequence
HVPLSRTVRCTCISISNQPVNPRSLEKLEIIPASQFCPRVEIIATMKKKGEKRCLNPESKAIKNLLKAVSKERSKASP

SEQ ID NO: 26 - protein, 78 aa, synthetic construct
  REGION 1..78: note = an artificially synthesized sequence
IVPLSRTVRCTCISISNQPVNPRSLEKLEIIPASQFCPRVEIIATMKKKGEKRCLNPESKAIKNLLKAVSKERSKASP

SEQ ID NO: 27 - protein, 78 aa, synthetic construct
  REGION 1..78: note = an artificially synthesized sequence
KVPLSRTVRCTCISISNQPVNPRSLEKLEIIPASQFCPRVEIIATMKKKGEKRCLNPESKAIKNLLKAVSKERSKASP

SEQ ID NO: 28 - protein, 78 aa, synthetic construct
  REGION 1..78: note = an artificially synthesized sequence
LVPLSRTVRCTCISISNQPVNPRSLEKLEIIPASQFCPRVEIIATMKKKGEKRCLNPESKAIKNLLKAVSKERSKASP

SEQ ID NO: 29 - protein, 78 aa, synthetic construct
  REGION 1..78: note = an artificially synthesized sequence
MVPLSRTVRCTCISISNQPVNPRSLEKLEIIPASQFCPRVEIIATMKKKGEKRCLNPESKAIKNLLKAVSKERSKASP

SEQ ID NO: 30 - protein, 78 aa, synthetic construct
  REGION 1..78: note = an artificially synthesized sequence
NVPLSRTVRCTCISISNQPVNPRSLEKLEIIPASQFCPRVEIIATMKKKGEKRCLNPESKAIKNLLKAVSKERSKASP

SEQ ID NO: 31 - protein, 78 aa, synthetic construct
  REGION 1..78: note = an artificially synthesized sequence
PVPLSRTVRCTCISISNQPVNPRSLEKLEIIPASQFCPRVEIIATMKKKGEKRCLNPESKAIKNLLKAVSKERSKASP

SEQ ID NO: 32 - protein, 78 aa, synthetic construct
  REGION 1..78: note = an artificially synthesized sequence
QVPLSRTVRCTCISISNQPVNPRSLEKLEIIPASQFCPRVEIIATMKKKGEKRCLNPESKAIKNLLKAVSKERSKASP

SEQ ID NO: 33 - protein, 78 aa, synthetic construct
  REGION 1..78: note = an artificially synthesized sequence
RVPLSRTVRCTCISISNQPVNPRSLEKLEIIPASQFCPRVEIIATMKKKGEKRCLNPESKAIKNLLKAVSKERSKASP

SEQ ID NO: 34 - protein, 78 aa, synthetic construct
  REGION 1..78: note = an artificially synthesized sequence
SVPLSRTVRCTCISISNQPVNPRSLEKLEIIPASQFCPRVEIIATMKKKGEKRCLNPESKAIKNLLKAVSKERSKASP

SEQ ID NO: 35 - protein, 78 aa, synthetic construct
  REGION 1..78: note = an artificially synthesized sequence
TVPLSRTVRCTCISISNQPVNPRSLEKLEIIPASQFCPRVEIIATMKKKGEKRCLNPESKAIKNLLKAVSKERSKASP

SEQ ID NO: 36 - protein, 78 aa, synthetic construct
  REGION 1..78: note = an artificially synthesized sequence
VVPLSRTVRCTCISISNQPVNPRSLEKLEIIPASQFCPRVEIIATMKKKGEKRCLNPESKAIKNLLKAVSKERSKASP

SEQ ID NO: 37 - protein, 78 aa, synthetic construct
  REGION 1..78: note = an artificially synthesized sequence
WVPLSRTVRCTCISISNQPVNPRSLEKLEIIPASQFCPRVEIIATMKKKGEKRCLNPESKAIKNLLKAVSKERSKASP

SEQ ID NO: 38 - protein, 78 aa, synthetic construct
  REGION 1..78: note = an artificially synthesized sequence
YVPLSRTVRCTCISISNQPVNPRSLEKLEIIPASQFCPRVEIIATMKKKGEKRCLNPESKAIKNLLKAVSKERSKASP

SEQ ID NO: 39 - protein, 78 aa, synthetic construct
  REGION 1..78: note = an artificially synthesized sequence
VAPLSRTVRCTCISISNQPVNPRSLEKLEIIPASQFCPRVEIIATMKKKGEKRCLNPESKAIKNLLKAVSKERSKASP

SEQ ID NO: 40 - protein, 78 aa, synthetic construct
  REGION 1..78: note = an artificially synthesized sequence
VDPLSRTVRCTCISISNQPVNPRSLEKLEIIPASQFCPRVEIIATMKKKGEKRCLNPESKAIKNLLKAVSKERSKASP

SEQ ID NO: 41 - protein, 78 aa, synthetic construct
  REGION 1..78: note = an artificially synthesized sequence
VEPLSRTVRCTCISISNQPVNPRSLEKLEIIPASQFCPRVEIIATMKKKGEKRCLNPESKAIKNLLKAVSKERSKASP

SEQ ID NO: 42 - protein, 78 aa, synthetic construct
  REGION 1..78: note = an artificially synthesized sequence
VFPLSRTVRCTCISISNQPVNPRSLEKLEIIPASQFCPRVEIIATMKKKGEKRCLNPESKAIKNLLKAVSKERSKASP

SEQ ID NO: 43 - protein, 78 aa, synthetic construct
  REGION 1..78: note = an artificially synthesized sequence
VGPLSRTVRCTCISISNQPVNPRSLEKLEIIPASQFCPRVEIIATMKKKGEKRCLNPESKAIKNLLKAVSKERSKASP

SEQ ID NO: 44 - protein, 78 aa, synthetic construct
  REGION 1..78: note = an artificially synthesized sequence
VHPLSRTVRCTCISISNQPVNPRSLEKLEIIPASQFCPRVEIIATMKKKGEKRCLNPESKAIKNLLKAVSKERSKASP

SEQ ID NO: 45 - protein, 78 aa, synthetic construct
  REGION 1..78: note = an artificially synthesized sequence
VIPLSRTVRCTCISISNQPVNPRSLEKLEIIPASQFCPRVEIIATMKKKGEKRCLNPESKAIKNLLKAVSKERSKASP

SEQ ID NO: 46 - protein, 78 aa, synthetic construct
  REGION 1..78: note = an artificially synthesized sequence
VKPLSRTVRCTCISISNQPVNPRSLEKLEIIPASQFCPRVEIIATMKKKGEKRCLNPESKAIKNLLKAVSKERSKASP

SEQ ID NO: 47 - protein, 78 aa, synthetic construct
  REGION 1..78: note = an artificially synthesized sequence
VLPLSRTVRCTCISISNQPVNPRSLEKLEIIPASQFCPRVEIIATMKKKGEKRCLNPESKAIKNLLKAVSKERSKASP

SEQ ID NO: 48 - protein, 78 aa, synthetic construct
  REGION 1..78: note = an artificially synthesized sequence
VMPLSRTVRCTCISISNQPVNPRSLEKLEIIPASQFCPRVEIIATMKKKGEKRCLNPESKAIKNLLKAVSKERSKASP

SEQ ID NO: 49 - protein, 78 aa, synthetic construct
  REGION 1..78: note = an artificially synthesized sequence
VNPLSRTVRCTCISISNQPVNPRSLEKLEIIPASQFCPRVEIIATMKKKGEKRCLNPESKAIKNLLKAVSKERSKASP

SEQ ID NO: 50 - protein, 78 aa, synthetic construct
  REGION 1..78: note = an artificially synthesized sequence
VPPLSRTVRCTCISISNQPVNPRSLEKLEIIPASQFCPRVEIIATMKKKGEKRCLNPESKAIKNLLKAVSKERSKASP

SEQ ID NO: 51 - protein, 78 aa, synthetic construct
  REGION 1..78: note = an artificially synthesized sequence
VQPLSRTVRCTCISISNQPVNPRSLEKLEIIPASQFCPRVEIIATMKKKGEKRCLNPESKAIKNLLKAVSKERSKASP

SEQ ID NO: 52 - protein, 78 aa, synthetic construct
  REGION 1..78: note = an artificially synthesized sequence
VRPLSRTVRCTCISISNQPVNPRSLEKLEIIPASQFCPRVEIIATMKKKGEKRCLNPESKAIKNLLKAVSKERSKASP

SEQ ID NO: 53 - protein, 78 aa, synthetic construct
  REGION 1..78: note = an artificially synthesized sequence
VSPLSRTVRCTCISISNQPVNPRSLEKLEIIPASQFCPRVEIIATMKKKGEKRCLNPESKAIKNLLKAVSKERSKASP

SEQ ID NO: 54 - protein, 78 aa, synthetic construct
  REGION 1..78: note = an artificially synthesized sequence
VTPLSRTVRCTCISISNQPVNPRSLEKLEIIPASQFCPRVEIIATMKKKGEKRCLNPESKAIKNLLKAVSKERSKASP

SEQ ID NO: 55 - protein, 78 aa, synthetic construct; identical to SEQ ID NO: 36
  REGION 1..78: note = an artificially synthesized sequence

SEQ ID NO: 56 - protein, 78 aa, synthetic construct
  REGION 1..78: note = an artificially synthesized sequence
VWPLSRTVRCTCISISNQPVNPRSLEKLEIIPASQFCPRVEIIATMKKKGEKRCLNPESKAIKNLLKAVSKERSKASP

SEQ ID NO: 57 - protein, 78 aa, synthetic construct
  REGION 1..78: note = an artificially synthesized sequence
VYPLSRTVRCTCISISNQPVNPRSLEKLEIIPASQFCPRVEIIATMKKKGEKRCLNPESKAIKNLLKAVSKERSKASP

SEQ ID NO: 58 - protein, 230 aa, synthetic construct
  REGION 1..230: note = an artificially synthesized sequence
EPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 59 - protein, 230 aa, synthetic construct
  REGION 1..230: note = an artificially synthesized sequence
EPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRCEMTKNQVSLSCAVKGFYPSDIAVEWESNGQPENNYKTTPPVLDSDGSFFLVSKLTVDKSRWQEGNVFSCSVMHEALHNRYTQKSLSLSP

SEQ ID NO: 60 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 61 - protein, 73 aa, Homo sapiens
FPMFKRGRCLCIGPGVKAVKVADIEKASIMYPSNNCDKIEVIITLKENKGQRCLNPKSKQARLIIKKVERKNF

SEQ ID NO: 62 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 63 - protein, 77 aa, synthetic construct
  REGION 1..77: note = an artificially synthesized sequence
FPMFKRGRCLCIGPGVKAVKVADIEKLEIIPASQFCPRVEIIATMKKKGEKRCLNPESKAIKNLLKAVSKERSKASP

SEQ ID NO: 64 - protein, 4 aa, synthetic construct
  REGION 1..4: note = an artificially synthesized sequence
GGGS

SEQ ID NO: 65 - protein, 4 aa, synthetic construct
  REGION 1..4: note = an artificially synthesized sequence
GGSG

SEQ ID NO: 66 - protein, 4 aa, synthetic construct
  REGION 1..4: note = an artificially synthesized sequence
GSGG

SEQ ID NO: 67 - protein, 4 aa, synthetic construct
  REGION 1..4: note = an artificially synthesized sequence
SGGG

SEQ ID NO: 68 - protein, 4 aa, synthetic construct
  REGION 1..4: note = an artificially synthesized sequence
GSSG

SEQ ID NO: 69 - protein, 5 aa, synthetic construct
  REGION 1..5: note = an artificially synthesized sequence
GGGGS

SEQ ID NO: 70 - protein, 5 aa, synthetic construct
  REGION 1..5: note = an artificially synthesized sequence
GGGSG

SEQ ID NO: 71 - protein, 5 aa, synthetic construct
  REGION 1..5: note = an artificially synthesized sequence
GGSGG

SEQ ID NO: 72 - protein, 5 aa, synthetic construct
  REGION 1..5: note = an artificially synthesized sequence
GSGGG

SEQ ID NO: 73 - protein, 5 aa, synthetic construct
  REGION 1..5: note = an artificially synthesized sequence
GSGGS

SEQ ID NO: 74 - protein, 5 aa, synthetic construct
  REGION 1..5: note = an artificially synthesized sequence
SGGGG

SEQ ID NO: 75 - protein, 5 aa, synthetic construct
  REGION 1..5: note = an artificially synthesized sequence
GSSGG

SEQ ID NO: 76 - protein, 5 aa, synthetic construct
  REGION 1..5: note = an artificially synthesized sequence
GSGSG

SEQ ID NO: 77 - protein, 5 aa, synthetic construct
  REGION 1..5: note = an artificially synthesized sequence
SGGSG

SEQ ID NO: 78 - protein, 5 aa, synthetic construct
  REGION 1..5: note = an artificially synthesized sequence
GSSSG

SEQ ID NO: 79 - protein, 6 aa, synthetic construct
  REGION 1..6: note = an artificially synthesized sequence
GGGGGS

SEQ ID NO: 80 - protein, 6 aa, synthetic construct
  REGION 1..6: note = an artificially synthesized sequence
SGGGGG

SEQ ID NO: 81 - protein, 7 aa, synthetic construct
  REGION 1..7: note = an artificially synthesized sequence
GGGGGGS

SEQ ID NO: 82 - protein, 7 aa, synthetic construct
  REGION 1..7: note = an artificially synthesized sequence
SGGGGGG

SEQ ID NO: 83 - protein, 21 aa, synthetic construct
  REGION 1..21: note = an artificially synthesized sequence
MNQTAILICCLIFLTLSGIQG

SEQ ID NO: 84 - protein, 22 aa, synthetic construct
  REGION 1..22: note = an artificially synthesized sequence
MKKSGVLFLLGIILLVLIGVQG

SEQ ID NO: 85 - protein, 21 aa, synthetic construct
  REGION 1..21: note = an artificially synthesized sequence
MSVKGMAIALAVILCATVVQG

SEQ ID NO: 86 - protein, 8 aa, synthetic construct
  REGION 1..8: note = an artificially synthesized sequence
VPLSRTVR

SEQ ID NO: 87 - protein, 8 aa, synthetic construct
  REGION 1..8: note = an artificially synthesized sequence
VALSRTVR

SEQ ID NO: 88 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 89 - protein, 53 aa, synthetic construct
  REGION 1..53: note = an artificially synthesized sequence
MVLEVSDHQVLNDAEVAALLENFSSSYDYGENESDSCCTSPPCPQDFSLNFDR

SEQ ID NO: 90 - protein, 21 aa, synthetic construct
  REGION 1..21: note = an artificially synthesized sequence
NFSSSYDYGENESDSCCTSPP

SEQ ID NO: 91 - protein, 8 aa, synthetic construct
  REGION 1..8: note = an artificially synthesized sequence
FPMFKRGR

SEQ ID NO: 92 - protein, 73 aa, synthetic construct
  REGION 1..73: note = an artificially synthesized sequence
FAMFKRGRCLCIGPGVKAVKVADIEKASIMYPSNNCDKIEVIITLKENKGQRCLNPKSKQARLIIKKVERKNF

SEQ ID NO: 93 - protein, 73 aa, synthetic construct
  REGION 1..73: note = an artificially synthesized sequence
FDMFKRGRCLCIGPGVKAVKVADIEKASIMYPSNNCDKIEVIITLKENKGQRCLNPKSKQARLIIKKVERKNF

SEQ ID NO: 94 - protein, 73 aa, synthetic construct
  REGION 1..73: note = an artificially synthesized sequence
FEMFKRGRCLCIGPGVKAVKVADIEKASIMYPSNNCDKIEVIITLKENKGQRCLNPKSKQARLIIKKVERKNF

SEQ ID NO: 95 - protein, 73 aa, synthetic construct
  REGION 1..73: note = an artificially synthesized sequence
FFMFKRGRCLCIGPGVKAVKVADIEKASIMYPSNNCDKIEVIITLKENKGQRCLNPKSKQARLIIKKVERKNF

SEQ ID NO: 96 - protein, 73 aa, synthetic construct
  REGION 1..73: note = an artificially synthesized sequence
FGMFKRGRCLCIGPGVKAVKVADIEKASIMYPSNNCDKIEVIITLKENKGQRCLNPKSKQARLIIKKVERKNF

SEQ ID NO: 97 - protein, 73 aa, synthetic construct
  REGION 1..73: note = an artificially synthesized sequence
FHMFKRGRCLCIGPGVKAVKVADIEKASIMYPSNNCDKIEVIITLKENKGQRCLNPKSKQARLIIKKVERKNF

SEQ ID NO: 98 - protein, 73 aa, synthetic construct
  REGION 1..73: note = an artificially synthesized sequence
FIMFKRGRCLCIGPGVKAVKVADIEKASIMYPSNNCDKIEVIITLKENKGQRCLNPKSKQARLIIKKVERKNF

SEQ ID NO: 99 - protein, 73 aa, synthetic construct
  REGION 1..73: note = an artificially synthesized sequence
FKMFKRGRCLCIGPGVKAVKVADIEKASIMYPSNNCDKIEVIITLKENKGQRCLNPKSKQARLIIKKVERKNF

SEQ ID NO: 100 - protein, 73 aa, synthetic construct
  REGION 1..73: note = an artificially synthesized sequence
FLMFKRGRCLCIGPGVKAVKVADIEKASIMYPSNNCDKIEVIITLKENKGQRCLNPKSKQARLIIKKVERKNF

SEQ ID NO: 101 - protein, 73 aa, synthetic construct
  REGION 1..73: note = an artificially synthesized sequence
FMMFKRGRCLCIGPGVKAVKVADIEKASIMYPSNNCDKIEVIITLKENKGQRCLNPKSKQARLIIKKVERKNF

SEQ ID NO: 102 - protein, 73 aa, synthetic construct
  REGION 1..73: note = an artificially synthesized sequence
FNMFKRGRCLCIGPGVKAVKVADIEKASIMYPSNNCDKIEVIITLKENKGQRCLNPKSKQARLIIKKVERKNF

SEQ ID NO: 103 - protein, 73 aa, synthetic construct
  REGION 1..73: note = an artificially synthesized sequence
FQMFKRGRCLCIGPGVKAVKVADIEKASIMYPSNNCDKIEVIITLKENKGQRCLNPKSKQARLIIKKVERKNF

SEQ ID NO: 104 - protein, 73 aa, synthetic construct
  REGION 1..73: note = an artificially synthesized sequence
FRMFKRGRCLCIGPGVKAVKVADIEKASIMYPSNNCDKIEVIITLKENKGQRCLNPKSKQARLIIKKVERKNF

SEQ ID NO: 105 - protein, 73 aa, synthetic construct
  REGION 1..73: note = an artificially synthesized sequence
FSMFKRGRCLCIGPGVKAVKVADIEKASIMYPSNNCDKIEVIITLKENKGQRCLNPKSKQARLIIKKVERKNF

SEQ ID NO: 106 - protein, 73 aa, synthetic construct
  REGION 1..73: note = an artificially synthesized sequence
FTMFKRGRCLCIGPGVKAVKVADIEKASIMYPSNNCDKIEVIITLKENKGQRCLNPKSKQARLIIKKVERKNF

SEQ ID NO: 107 - protein, 73 aa, synthetic construct
  REGION 1..73: note = an artificially synthesized sequence
FVMFKRGRCLCIGPGVKAVKVADIEKASIMYPSNNCDKIEVIITLKENKGQRCLNPKSKQARLIIKKVERKNF

SEQ ID NO: 108 - protein, 73 aa, synthetic construct
  REGION 1..73: note = an artificially synthesized sequence
FWMFKRGRCLCIGPGVKAVKVADIEKASIMYPSNNCDKIEVIITLKENKGQRCLNPKSKQARLIIKKVERKNF

SEQ ID NO: 109 - protein, 73 aa, synthetic construct
  REGION 1..73: note = an artificially synthesized sequence
FYMFKRGRCLCIGPGVKAVKVADIEKASIMYPSNNCDKIEVIITLKENKGQRCLNPKSKQARLIIKKVERKNF

SEQ ID NO: 110 - protein, 74 aa, synthetic construct
  REGION 1..74: note = an artificially synthesized sequence
AFPMFKRGRCLCIGPGVKAVKVADIEKASIMYPSNNCDKIEVIITLKENKGQRCLNPKSKQARLIIKKVERKNF

SEQ ID NO: 111 - protein, 74 aa, synthetic construct
  REGION 1..74: note = an artificially synthesized sequence
DFPMFKRGRCLCIGPGVKAVKVADIEKASIMYPSNNCDKIEVIITLKENKGQRCLNPKSKQARLIIKKVERKNF

SEQ ID NO: 112 - protein, 74 aa, synthetic construct
  REGION 1..74: note = an artificially synthesized sequence
EFPMFKRGRCLCIGPGVKAVKVADIEKASIMYPSNNCDKIEVIITLKENKGQRCLNPKSKQARLIIKKVERKNF

SEQ ID NO: 113 - protein, 74 aa, synthetic construct
  REGION 1..74: note = an artificially synthesized sequence
FFPMFKRGRCLCIGPGVKAVKVADIEKASIMYPSNNCDKIEVIITLKENKGQRCLNPKSKQARLIIKKVERKNF

SEQ ID NO: 114 - protein, 74 aa, synthetic construct
  REGION 1..74: note = an artificially synthesized sequence
GFPMFKRGRCLCIGPGVKAVKVADIEKASIMYPSNNCDKIEVIITLKENKGQRCLNPKSKQARLIIKKVERKNF

SEQ ID NO: 115 - protein, 74 aa, synthetic construct
  REGION 1..74: note = an artificially synthesized sequence
HFPMFKRGRCLCIGPGVKAVKVADIEKASIMYPSNNCDKIEVIITLKENKGQRCLNPKSKQARLIIKKVERKNF

SEQ ID NO: 116 - protein, 74 aa, synthetic construct
  REGION 1..74: note = an artificially synthesized sequence
IFPMFKRGRCLCIGPGVKAVKVADIEKASIMYPSNNCDKIEVIITLKENKGQRCLNPKSKQARLIIKKVERKNF

SEQ ID NO: 117 - protein, 74 aa, synthetic construct
  REGION 1..74: note = an artificially synthesized sequence
KFPMFKRGRCLCIGPGVKAVKVADIEKASIMYPSNNCDKIEVIITLKENKGQRCLNPKSKQARLIIKKVERKNF

SEQ ID NO: 118 - protein, 74 aa, synthetic construct
  REGION 1..74: note = an artificially synthesized sequence
LFPMFKRGRCLCIGPGVKAVKVADIEKASIMYPSNNCDKIEVIITLKENKGQRCLNPKSKQARLIIKKVERKNF

SEQ ID NO: 119 - protein, 74 aa, synthetic construct
  REGION 1..74: note = an artificially synthesized sequence
MFPMFKRGRCLCIGPGVKAVKVADIEKASIMYPSNNCDKIEVIITLKENKGQRCLNPKSKQARLIIKKVERKNF

SEQ ID NO: 120 - protein, 74 aa, synthetic construct
  REGION 1..74: note = an artificially synthesized sequence
NFPMFKRGRCLCIGPGVKAVKVADIEKASIMYPSNNCDKIEVIITLKENKGQRCLNPKSKQARLIIKKVERKNF

SEQ ID NO: 121 - protein, 74 aa, synthetic construct
  REGION 1..74: note = an artificially synthesized sequence
PFPMFKRGRCLCIGPGVKAVKVADIEKASIMYPSNNCDKIEVIITLKENKGQRCLNPKSKQARLIIKKVERKNF

SEQ ID NO: 122 - protein, 74 aa, synthetic construct
  REGION 1..74: note = an artificially synthesized sequence
QFPMFKRGRCLCIGPGVKAVKVADIEKASIMYPSNNCDKIEVIITLKENKGQRCLNPKSKQARLIIKKVERKNF

SEQ ID NO: 123 - protein, 74 aa, synthetic construct
  REGION 1..74: note = an artificially synthesized sequence
RFPMFKRGRCLCIGPGVKAVKVADIEKASIMYPSNNCDKIEVIITLKENKGQRCLNPKSKQARLIIKKVERKNF

SEQ ID NO: 124 - protein, 74 aa, synthetic construct
  REGION 1..74: note = an artificially synthesized sequence
SFPMFKRGRCLCIGPGVKAVKVADIEKASIMYPSNNCDKIEVIITLKENKGQRCLNPKSKQARLIIKKVERKNF

SEQ ID NO: 125 - protein, 74 aa, synthetic construct
  REGION 1..74: note = an artificially synthesized sequence
TFPMFKRGRCLCIGPGVKAVKVADIEKASIMYPSNNCDKIEVIITLKENKGQRCLNPKSKQARLIIKKVERKNF

SEQ ID NO: 126 - protein, 74 aa, synthetic construct
  REGION 1..74: note = an artificially synthesized sequence
VFPMFKRGRCLCIGPGVKAVKVADIEKASIMYPSNNCDKIEVIITLKENKGQRCLNPKSKQARLIIKKVERKNF

SEQ ID NO: 127 - protein, 74 aa, synthetic construct
  REGION 1..74: note = an artificially synthesized sequence
WFPMFKRGRCLCIGPGVKAVKVADIEKASIMYPSNNCDKIEVIITLKENKGQRCLNPKSKQARLIIKKVERKNF

SEQ ID NO: 128 - protein, 74 aa, synthetic construct
  REGION 1..74: note = an artificially synthesized sequence
YFPMFKRGRCLCIGPGVKAVKVADIEKASIMYPSNNCDKIEVIITLKENKGQRCLNPKSKQARLIIKKVERKNF

SEQ ID NO: 129 - protein, 74 aa, synthetic construct
  REGION 1..74: note = an artificially synthesized sequence
FAPMFKRGRCLCIGPGVKAVKVADIEKASIMYPSNNCDKIEVIITLKENKGQRCLNPKSKQARLIIKKVERKNF

SEQ ID NO: 130 - protein, 74 aa, synthetic construct
  REGION 1..74: note = an artificially synthesized sequence
FDPMFKRGRCLCIGPGVKAVKVADIEKASIMYPSNNCDKIEVIITLKENKGQRCLNPKSKQARLIIKKVERKNF

SEQ ID NO: 131 - protein, 74 aa, synthetic construct
  REGION 1..74: note = an artificially synthesized sequence
FEPMFKRGRCLCIGPGVKAVKVADIEKASIMYPSNNCDKIEVIITLKENKGQRCLNPKSKQARLIIKKVERKNF

SEQ ID NO: 132 - protein, 74 aa, synthetic construct; identical to SEQ ID NO: 113
  REGION 1..74: note = an artificially synthesized sequence

SEQ ID NO: 133 - protein, 74 aa, synthetic construct
  REGION 1..74: note = an artificially synthesized sequence
FGPMFKRGRCLCIGPGVKAVKVADIEKASIMYPSNNCDKIEVIITLKENKGQRCLNPKSKQARLIIKKVERKNF

SEQ ID NO: 134 - protein, 74 aa, synthetic construct
  REGION 1..74: note = an artificially synthesized sequence
FHPMFKRGRCLCIGPGVKAVKVADIEKASIMYPSNNCDKIEVIITLKENKGQRCLNPKSKQARLIIKKVERKNF

SEQ ID NO: 135 - protein, 74 aa, synthetic construct
  REGION 1..74: note = an artificially synthesized sequence
FIPMFKRGRCLCIGPGVKAVKVADIEKASIMYPSNNCDKIEVIITLKENKGQRCLNPKSKQARLIIKKVERKNF

SEQ ID NO: 136 - protein, 74 aa, synthetic construct
  REGION 1..74: note = an artificially synthesized sequence
FKPMFKRGRCLCIGPGVKAVKVADIEKASIMYPSNNCDKIEVIITLKENKGQRCLNPKSKQARLIIKKVERKNF

SEQ ID NO: 137 - protein, 74 aa, synthetic construct
  REGION 1..74: note = an artificially synthesized sequence
FLPMFKRGRCLCIGPGVKAVKVADIEKASIMYPSNNCDKIEVIITLKENKGQRCLNPKSKQARLIIKKVERKNF

SEQ ID NO: 138 - protein, 74 aa, synthetic construct
  REGION 1..74: note = an artificially synthesized sequence
FMPMFKRGRCLCIGPGVKAVKVADIEKASIMYPSNNCDKIEVIITLKENKGQRCLNPKSKQARLIIKKVERKNF

SEQ ID NO: 139 - protein, 74 aa, synthetic construct
  REGION 1..74: note = an artificially synthesized sequence
FNPMFKRGRCLCIGPGVKAVKVADIEKASIMYPSNNCDKIEVIITLKENKGQRCLNPKSKQARLIIKKVERKNF

SEQ ID NO: 140 - protein, 74 aa, synthetic construct
  REGION 1..74: note = an artificially synthesized sequence
FPPMFKRGRCLCIGPGVKAVKVADIEKASIMYPSNNCDKIEVIITLKENKGQRCLNPKSKQARLIIKKVERKNF

SEQ ID NO: 141 - protein, 74 aa, synthetic construct
  REGION 1..74: note = an artificially synthesized sequence
FQPMFKRGRCLCIGPGVKAVKVADIEKASIMYPSNNCDKIEVIITLKENKGQRCLNPKSKQARLIIKKVERKNF

SEQ ID NO: 142 - protein, 74 aa, synthetic construct
  REGION 1..74: note = an artificially synthesized sequence
FRPMFKRGRCLCIGPGVKAVKVADIEKASIMYPSNNCDKIEVIITLKENKGQRCLNPKSKQARLIIKKVERKNF

SEQ ID NO: 143 - protein, 74 aa, synthetic construct
  REGION 1..74: note = an artificially synthesized sequence
FSPMFKRGRCLCIGPGVKAVKVADIEKASIMYPSNNCDKIEVIITLKENKGQRCLNPKSKQARLIIKKVERKNF

SEQ ID NO: 144 - protein, 74 aa, synthetic construct
  REGION 1..74: note = an artificially synthesized sequence
FTPMFKRGRCLCIGPGVKAVKVADIEKASIMYPSNNCDKIEVIITLKENKGQRCLNPKSKQARLIIKKVERKNF

SEQ ID NO: 145 - protein, 74 aa, synthetic construct
  REGION 1..74: note = an artificially synthesized sequence
FVPMFKRGRCLCIGPGVKAVKVADIEKASIMYPSNNCDKIEVIITLKENKGQRCLNPKSKQARLIIKKVERKNF

SEQ ID NO: 146 - protein, 74 aa, synthetic construct
  REGION 1..74: note = an artificially synthesized sequence
FWPMFKRGRCLCIGPGVKAVKVADIEKASIMYPSNNCDKIEVIITLKENKGQRCLNPKSKQARLIIKKVERKNF

SEQ ID NO: 147 - protein, 74 aa, synthetic construct
  REGION 1..74: note = an artificially synthesized sequence
FYPMFKRGRCLCIGPGVKAVKVADIEKASIMYPSNNCDKIEVIITLKENKGQRCLNPKSKQARLIIKKVERKNF

SEQ ID NO: 148 - protein, 19 aa, synthetic construct
  REGION 1..19: note = an artificially synthesized sequence
MGWSCIILFLVATATGVHS

SEQ ID NO: 149 - protein, 77 aa, synthetic construct
  REGION 1..77: note = an artificially synthesized sequence
FAMFKRGRCLCIGPGVKAVKVADIEKLEIIPASQFCPRVEIIATMKKKGEKRCLNPESKAIKNLLKAVSKERSKASP

SEQ ID NO: 150 - protein, 77 aa, synthetic construct
  REGION 1..77: note = an artificially synthesized sequence
FDMFKRGRCLCIGPGVKAVKVADIEKLEIIPASQFCPRVEIIATMKKKGEKRCLNPESKAIKNLLKAVSKERSKASP

SEQ ID NO: 151 - protein, 77 aa, synthetic construct
  REGION 1..77: note = an artificially synthesized sequence
FEMFKRGRCLCIGPGVKAVKVADIEKLEIIPASQFCPRVEIIATMKKKGEKRCLNPESKAIKNLLKAVSKERSKASP

SEQ ID NO: 152 - protein, 77 aa, synthetic construct
  REGION 1..77: note = an artificially synthesized sequence
FFMFKRGRCLCIGPGVKAVKVADIEKLEIIPASQFCPRVEIIATMKKKGEKRCLNPESKAIKNLLKAVSKERSKASP

SEQ ID NO: 153 - protein, 77 aa, synthetic construct
  REGION 1..77: note = an artificially synthesized sequence
FGMFKRGRCLCIGPGVKAVKVADIEKLEIIPASQFCPRVEIIATMKKKGEKRCLNPESKAIKNLLKAVSKERSKASP

SEQ ID NO: 154 - protein, 77 aa, synthetic construct
  REGION 1..77: note = an artificially synthesized sequence
FHMFKRGRCLCIGPGVKAVKVADIEKLEIIPASQFCPRVEIIATMKKKGEKRCLNPESKAIKNLLKAVSKERSKASP

SEQ ID NO: 155 - protein, 77 aa, synthetic construct
  REGION 1..77: note = an artificially synthesized sequence
FIMFKRGRCLCIGPGVKAVKVADIEKLEIIPASQFCPRVEIIATMKKKGEKRCLNPESKAIKNLLKAVSKERSKASP

SEQ ID NO: 156 - protein, 77 aa, synthetic construct
  REGION 1..77: note = an artificially synthesized sequence
FKMFKRGRCLCIGPGVKAVKVADIEKLEIIPASQFCPRVEIIATMKKKGEKRCLNPESKAIKNLLKAVSKERSKASP

SEQ ID NO: 157 - protein, 77 aa, synthetic construct
  REGION 1..77: note = an artificially synthesized sequence
FLMFKRGRCLCIGPGVKAVKVADIEKLEIIPASQFCPRVEIIATMKKKGEKRCLNPESKAIKNLLKAVSKERSKASP

SEQ ID NO: 158 - protein, 77 aa, synthetic construct
  REGION 1..77: note = an artificially synthesized sequence
FMMFKRGRCLCIGPGVKAVKVADIEKLEIIPASQFCPRVEIIATMKKKGEKRCLNPESKAIKNLLKAVSKERSKASP

SEQ ID NO: 159 - protein, 77 aa, synthetic construct
  REGION 1..77: note = an artificially synthesized sequence
FNMFKRGRCLCIGPGVKAVKVADIEKLEIIPASQFCPRVEIIATMKKKGEKRCLNPESKAIKNLLKAVSKERSKASP

SEQ ID NO: 160 - protein, 77 aa, synthetic construct
  REGION 1..77: note = an artificially synthesized sequence
FQMFKRGRCLCIGPGVKAVKVADIEKLEIIPASQFCPRVEIIATMKKKGEKRCLNPESKAIKNLLKAVSKERSKASP

SEQ ID NO: 161 - protein, 77 aa, synthetic construct
  REGION 1..77: note = an artificially synthesized sequence
FRMFKRGRCLCIGPGVKAVKVADIEKLEIIPASQFCPRVEIIATMKKKGEKRCLNPESKAIKNLLKAVSKERSKASP

SEQ ID NO: 162 - protein, 77 aa, synthetic construct
  REGION 1..77: note = an artificially synthesized sequence
FSMFKRGRCLCIGPGVKAVKVADIEKLEIIPASQFCPRVEIIATMKKKGEKRCLNPESKAIKNLLKAVSKERSKASP

SEQ ID NO: 163 - protein, 77 aa, synthetic construct
  REGION 1..77: note = an artificially synthesized sequence
FTMFKRGRCLCIGPGVKAVKVADIEKLEIIPASQFCPRVEIIATMKKKGEKRCLNPESKAIKNLLKAVSKERSKASP

SEQ ID NO: 164 - protein, 77 aa, synthetic construct
  REGION 1..77: note = an artificially synthesized sequence
FVMFKRGRCLCIGPGVKAVKVADIEKLEIIPASQFCPRVEIIATMKKKGEKRCLNPESKAIKNLLKAVSKERSKASP

SEQ ID NO: 165 - protein, 77 aa, synthetic construct
  REGION 1..77: note = an artificially synthesized sequence
FWMFKRGRCLCIGPGVKAVKVADIEKLEIIPASQFCPRVEIIATMKKKGEKRCLNPESKAIKNLLKAVSKERSKASP

SEQ ID NO: 166 - protein, 77 aa, synthetic construct
  REGION 1..77: note = an artificially synthesized sequence
FYMFKRGRCLCIGPGVKAVKVADIEKLEIIPASQFCPRVEIIATMKKKGEKRCLNPESKAIKNLLKAVSKERSKASP

SEQ ID NO: 167 - protein, 78 aa, synthetic construct
  REGION 1..78: note = an artificially synthesized sequence
AFPMFKRGRCLCIGPGVKAVKVADIEKLEIIPASQFCPRVEIIATMKKKGEKRCLNPESKAIKNLLKAVSKERSKASP

SEQ ID NO: 168 - protein, 78 aa, synthetic construct
  REGION 1..78: note = an artificially synthesized sequence
DFPMFKRGRCLCIGPGVKAVKVADIEKLEIIPASQFCPRVEIIATMKKKGEKRCLNPESKAIKNLLKAVSKERSKASP

SEQ ID NO: 169 - protein, 78 aa, synthetic construct
  REGION 1..78: note = an artificially synthesized sequence
EFPMFKRGRCLCIGPGVKAVKVADIEKLEIIPASQFCPRVEIIATMKKKGEKRCLNPESKAIKNLLKAVSKERSKASP

SEQ ID NO: 170 - protein, 78 aa, synthetic construct
  REGION 1..78: note = an artificially synthesized sequence
FFPMFKRGRCLCIGPGVKAVKVADIEKLEIIPASQFCPRVEIIATMKKKGEKRCLNPESKAIKNLLKAVSKERSKASP

SEQ ID NO: 171 - protein, 78 aa, synthetic construct
  REGION 1..78: note = an artificially synthesized sequence
GFPMFKRGRCLCIGPGVKAVKVADIEKLEIIPASQFCPRVEIIATMKKKGEKRCLNPESKAIKNLLKAVSKERSKASP

SEQ ID NO: 172 - protein, 78 aa, synthetic construct
  REGION 1..78: note = an artificially synthesized sequence
HFPMFKRGRCLCIGPGVKAVKVADIEKLEIIPASQFCPRVEIIATMKKKGEKRCLNPESKAIKNLLKAVSKERSKASP

SEQ ID NO: 173 - protein, 78 aa, synthetic construct
  REGION 1..78: note = an artificially synthesized sequence
IFPMFKRGRCLCIGPGVKAVKVADIEKLEIIPASQFCPRVEIIATMKKKGEKRCLNPESKAIKNLLKAVSKERSKASP

SEQ ID NO: 174 - protein, 78 aa, synthetic construct
  REGION 1..78: note = an artificially synthesized sequence
KFPMFKRGRCLCIGPGVKAVKVADIEKLEIIPASQFCPRVEIIATMKKKGEKRCLNPESKAIKNLLKAVSKERSKASP

SEQ ID NO: 175 - protein, 78 aa, synthetic construct
  REGION 1..78: note = an artificially synthesized sequence
LFPMFKRGRCLCIGPGVKAVKVADIEKLEIIPASQFCPRVEIIATMKKKGEKRCLNPESKAIKNLLKAVSKERSKASP

SEQ ID NO: 176 - protein, 78 aa, synthetic construct
  REGION 1..78: note = an artificially synthesized sequence
MFPMFKRGRCLCIGPGVKAVKVADIEKLEIIPASQFCPRVEIIATMKKKGEKRCLNPESKAIKNLLKAVSKERSKASP

SEQ ID NO: 177 - protein, 78 aa, synthetic construct
  REGION 1..78: note = an artificially synthesized sequence
NFPMFKRGRCLCIGPGVKAVKVADIEKLEIIPASQFCPRVEIIATMKKKGEKRCLNPESKAIKNLLKAVSKERSKASP

SEQ ID NO: 178 - protein, 78 aa, synthetic construct
  REGION 1..78: note = an artificially synthesized sequence
PFPMFKRGRCLCIGPGVKAVKVADIEKLEIIPASQFCPRVEIIATMKKKGEKRCLNPESKAIKNLLKAVSKERSKASP

SEQ ID NO: 179 - protein, 78 aa, synthetic construct
  REGION 1..78: note = an artificially synthesized sequence
QFPMFKRGRCLCIGPGVKAVKVADIEKLEIIPASQFCPRVEIIATMKKKGEKRCLNPESKAIKNLLKAVSKERSKASP

SEQ ID NO: 180 - protein, 78 aa, synthetic construct
  REGION 1..78: note = an artificially synthesized sequence
RFPMFKRGRCLCIGPGVKAVKVADIEKLEIIPASQFCPRVEIIATMKKKGEKRCLNPESKAIKNLLKAVSKERSKASP

SEQ ID NO: 181 - protein, 78 aa, synthetic construct
  REGION 1..78: note = an artificially synthesized sequence
SFPMFKRGRCLCIGPGVKAVKVADIEKLEIIPASQFCPRVEIIATMKKKGEKRCLNPESKAIKNLLKAVSKERSKASP

SEQ ID NO: 182 - protein, 78 aa, synthetic construct
  REGION 1..78: note = an artificially synthesized sequence
TFPMFKRGRCLCIGPGVKAVKVADIEKLEIIPASQFCPRVEIIATMKKKGEKRCLNPESKAIKNLLKAVSKERSKASP

SEQ ID NO: 183 - protein, 78 aa, synthetic construct
  REGION 1..78: note = an artificially synthesized sequence
VFPMFKRGRCLCIGPGVKAVKVADIEKLEIIPASQFCPRVEIIATMKKKGEKRCLNPESKAIKNLLKAVSKERSKASP

SEQ ID NO: 184 - protein, 78 aa, synthetic construct
  REGION 1..78: note = an artificially synthesized sequence
WFPMFKRGRCLCIGPGVKAVKVADIEKLEIIPASQFCPRVEIIATMKKKGEKRCLNPESKAIKNLLKAVSKERSKASP

SEQ ID NO: 185 - protein, 78 aa, synthetic construct
  REGION 1..78: note = an artificially synthesized sequence
YFPMFKRGRCLCIGPGVKAVKVADIEKLEIIPASQFCPRVEIIATMKKKGEKRCLNPESKAIKNLLKAVSKERSKASP

SEQ ID NO: 186 - protein, 78 aa, synthetic construct
  REGION 1..78: note = an artificially synthesized sequence
FAPMFKRGRCLCIGPGVKAVKVADIEKLEIIPASQFCPRVEIIATMKKKGEKRCLNPESKAIKNLLKAVSKERSKASP

SEQ ID NO: 187 - protein, 78 aa, synthetic construct
  REGION 1..78: note = an artificially synthesized sequence
FDPMFKRGRCLCIGPGVKAVKVADIEKLEIIPASQFCPRVEIIATMKKKGEKRCLNPESKAIKNLLKAVSKERSKASP

SEQ ID NO: 188 - protein, 78 aa, synthetic construct
  REGION 1..78: note = an artificially synthesized sequence
FEPMFKRGRCLCIGPGVKAVKVADIEKLEIIPASQFCPRVEIIATMKKKGEKRCLNPESKAIKNLLKAVSKERSKASP

SEQ ID NO: 189 - protein, 78 aa, synthetic construct; identical to SEQ ID NO: 170
  REGION 1..78: note = an artificially synthesized sequence

SEQ ID NO: 190 - protein, 78 aa, synthetic construct
  REGION 1..78: note = an artificially synthesized sequence
FGPMFKRGRCLCIGPGVKAVKVADIEKLEIIPASQFCPRVEIIATMKKKGEKRCLNPESKAIKNLLKAVSKERSKASP

SEQ ID NO: 191 - protein, 78 aa, synthetic construct
  REGION 1..78: note = an artificially synthesized sequence
FHPMFKRGRCLCIGPGVKAVKVADIEKLEIIPASQFCPRVEIIATMKKKGEKRCLNPESKAIKNLLKAVSKERSKASP

SEQ ID NO: 192 - protein, 78 aa, synthetic construct
  REGION 1..78: note = an artificially synthesized sequence
FIPMFKRGRCLCIGPGVKAVKVADIEKLEIIPASQFCPRVEIIATMKKKGEKRCLNPESKAIKNLLKAVSKERSKASP

SEQ ID NO: 193 - protein, 78 aa, synthetic construct
  REGION 1..78: note = an artificially synthesized sequence
FKPMFKRGRCLCIGPGVKAVKVADIEKLEIIPASQFCPRVEIIATMKKKGEKRCLNPESKAIKNLLKAVSKERSKASP

SEQ ID NO: 194 - protein, 78 aa, synthetic construct
  REGION 1..78: note = an artificially synthesized sequence
FLPMFKRGRCLCIGPGVKAVKVADIEKLEIIPASQFCPRVEIIATMKKKGEKRCLNPESKAIKNLLKAVSKERSKASP

SEQ ID NO: 195 - protein, 78 aa, synthetic construct
  REGION 1..78: note = an artificially synthesized sequence
FMPMFKRGRCLCIGPGVKAVKVADIEKLEIIPASQFCPRVEIIATMKKKGEKRCLNPESKAIKNLLKAVSKERSKASP

SEQ ID NO: 196 - protein, 78 aa, synthetic construct
  REGION 1..78: note = an artificially synthesized sequence
FNPMFKRGRCLCIGPGVKAVKVADIEKLEIIPASQFCPRVEIIATMKKKGEKRCLNPESKAIKNLLKAVSKERSKASP

SEQ ID NO: 197 - protein, 78 aa, synthetic construct
  REGION 1..78: note = an artificially synthesized sequence
FPPMFKRGRCLCIGPGVKAVKVADIEKLEIIPASQFCPRVEIIATMKKKGEKRCLNPESKAIKNLLKAVSKERSKASP

SEQ ID NO: 198 - protein, 78 aa, synthetic construct
  REGION 1..78: note = an artificially synthesized sequence
FQPMFKRGRCLCIGPGVKAVKVADIEKLEIIPASQFCPRVEIIATMKKKGEKRCLNPESKAIKNLLKAVSKERSKASP

SEQ ID NO: 199 - protein, 78 aa, synthetic construct
  REGION 1..78: note = an artificially synthesized sequence
FRPMFKRGRCLCIGPGVKAVKVADIEKLEIIPASQFCPRVEIIATMKKKGEKRCLNPESKAIKNLLKAVSKERSKASP

SEQ ID NO: 200 - protein, 78 aa, synthetic construct
  REGION 1..78: note = an artificially synthesized sequence
FSPMFKRGRCLCIGPGVKAVKVADIEKLEIIPASQFCPRVEIIATMKKKGEKRCLNPESKAIKNLLKAVSKERSKASP

SEQ ID NO: 201 - protein, 78 aa, synthetic construct
  REGION 1..78: note = an artificially synthesized sequence
FTPMFKRGRCLCIGPGVKAVKVADIEKLEIIPASQFCPRVEIIATMKKKGEKRCLNPESKAIKNLLKAVSKERSKASP

SEQ ID NO: 202 - protein, 78 aa, synthetic construct
  REGION 1..78: note = an artificially synthesized sequence
FVPMFKRGRCLCIGPGVKAVKVADIEKLEIIPASQFCPRVEIIATMKKKGEKRCLNPESKAIKNLLKAVSKERSKASP

SEQ ID NO: 203 - protein, 78 aa, synthetic construct
  REGION 1..78: note = an artificially synthesized sequence
FWPMFKRGRCLCIGPGVKAVKVADIEKLEIIPASQFCPRVEIIATMKKKGEKRCLNPESKAIKNLLKAVSKERSKASP

SEQ ID NO: 204 - protein, 78 aa, synthetic construct
  REGION 1..78: note = an artificially synthesized sequence
FYPMFKRGRCLCIGPGVKAVKVADIEKLEIIPASQFCPRVEIIATMKKKGEKRCLNPESKAIKNLLKAVSKERSKASP

SEQ ID NO: 205 - protein, 8 aa, synthetic construct
  REGION 1..8: note = an artificially synthesized sequence
  SITE 2: note = X is F, G, I, K, L, M, T, V, W, or Y
VXLSRTVR

SEQ ID NO: 206 - protein, 9 aa, synthetic construct
  REGION 1..9: note = an artificially synthesized sequence
  SITE 1: note = X is A, F, G, H, I, K, L, M, N, P, Q, R, S, T, V, W, or Y
XVPLSRTVR

SEQ ID NO: 207 - protein, 9 aa, synthetic construct
  REGION 1..9: note = an artificially synthesized sequence
  SITE 2: note = X is A, F, G, H, I, K, L, M, P, Q, R, S, T, V, W, or Y
VXPLSRTVR

SEQ ID NO: 208 - protein, 7 aa, synthetic construct
  REGION 1..7: note = an artificially synthesized sequence
PLSRTVR

SEQ ID NO: 209 - protein, 9 aa, synthetic construct
  REGION 1..9: note = an artificially synthesized sequence
  SITE 1: note = X is A, D, E, F, G, H, I, K, L, M, N, P, Q, S, T, V, W, or Y
XFPMFKRGR

SEQ ID NO: 210 - protein, 8 aa, synthetic construct
  REGION 1..8: note = an artificially synthesized sequence
  SITE 2: note = X is A, D, E, G, H, I, M, N, Q, R, S, T, V, W, or Y
FXMFKRGR

SEQ ID NO: 211 - protein, 9 aa, synthetic construct
  REGION 1..9: note = an artificially synthesized sequence
  SITE 2: note = X is A, D, E, F, G, H, L, M, N, P, Q, S, T, W, or Y
FXPMFKRGR

SEQ ID NO: 212 - protein, 8 aa, synthetic construct
  REGION 1..8: note = an artificially synthesized sequence
  SITE 2: note = X is F, G, I, L, M, T, V, W, or Y
VXLSRTVR

SEQ ID NO: 213 - protein, 9 aa, synthetic construct
  REGION 1..9: note = an artificially synthesized sequence
  SITE 1: note = X is A, G, I, L, N, Q, S, T, or W
XVPLSRTVR

SEQ ID NO: 214 - protein, 9 aa, synthetic construct
  REGION 1..9: note = an artificially synthesized sequence
  SITE 2: note = X is A, F, G, I, M, P, T, or V
VXPLSRTVR

SEQ ID NO: 215 - protein, 9 aa, synthetic construct
  REGION 1..9: note = an artificially synthesized sequence
  SITE 1: note = X is A, D, E, G, M, N, Q, S, T, V, or Y
XFPMFKRGR

SEQ ID NO: 216 - protein, 18 aa, synthetic construct
  REGION 1..18: note = an artificially synthesized sequence
FLTLSGIQGVPLSRTVRC

SEQ ID NO: 217 - protein, 18 aa, synthetic construct
  REGION 1..18: note = an artificially synthesized sequence
VATATGVHSFPMFKRGRC

SEQ ID NO: 218 - protein, 4 aa, synthetic construct
  REGION 1..4: note = an artificially synthesized sequence
VPLS

SEQ ID NO: 219 - protein, 4 aa, synthetic construct
  REGION 1..4: note = an artificially synthesized sequence
VALS

SEQ ID NO: 220 - protein, 4 aa, synthetic construct
  REGION 1..4: note = an artificially synthesized sequence
VDLS

SEQ ID NO: 221 - protein, 4 aa, synthetic construct
  REGION 1..4: note = an artificially synthesized sequence
VELS

SEQ ID NO: 222 - protein, 4 aa, synthetic construct
  REGION 1..4: note = an artificially synthesized sequence
VFLS

SEQ ID NO: 223 - protein, 4 aa, synthetic construct
  REGION 1..4: note = an artificially synthesized sequence
VGLS

SEQ ID NO: 224 - protein, 4 aa, synthetic construct
  REGION 1..4: note = an artificially synthesized sequence
VHLS

SEQ ID NO: 225 - protein, 4 aa, synthetic construct
  REGION 1..4: note = an artificially synthesized sequence
VILS

SEQ ID NO: 226 - protein, 4 aa, synthetic construct
  REGION 1..4: note = an artificially synthesized sequence
VKLS

SEQ ID NO: 227 - protein, 4 aa, synthetic construct
  REGION 1..4: note = an artificially synthesized sequence
VLLS

SEQ ID NO: 228 - protein, 4 aa, synthetic construct
  REGION 1..4: note = an artificially synthesized sequence
VMLS

SEQ ID NO: 229 - protein, 4 aa, synthetic construct
  REGION 1..4: note = an artificially synthesized sequence
VNLS

SEQ ID NO: 230 - protein, 4 aa, synthetic construct
  REGION 1..4: note = an artificially synthesized sequence
VQLS

SEQ ID NO: 231 - protein, 4 aa, synthetic construct
  REGION 1..4: note = an artificially synthesized sequence
VRLS

SEQ ID NO: 232 - protein, 4 aa, synthetic construct
  REGION 1..4: note = an artificially synthesized sequence
VSLS

SEQ ID NO: 233 - protein, 4 aa, synthetic construct
  REGION 1..4: note = an artificially synthesized sequence
VTLS

SEQ ID NO: 234 - protein, 4 aa, synthetic construct
  REGION 1..4: note = an artificially synthesized sequence
VVLS

SEQ ID NO: 235 - protein, 4 aa, synthetic construct
  REGION 1..4: note = an artificially synthesized sequence
VWLS

SEQ ID NO: 236 - protein, 4 aa, synthetic construct
  REGION 1..4: note = an artificially synthesized sequence
VYLS

SEQ ID NO: 237 - protein, 4 aa, synthetic construct
  REGION 1..4: note = an artificially synthesized sequence
AVPL

SEQ ID NO: 238 - protein, 4 aa, synthetic construct
  REGION 1..4: note = an artificially synthesized sequence
DVPL

SEQ ID NO: 239 - protein, 4 aa, synthetic construct
  REGION 1..4: note = an artificially synthesized sequence
EVPL

SEQ ID NO: 240 - protein, 4 aa, synthetic construct
  REGION 1..4: note = an artificially synthesized sequence
FVPL

SEQ ID NO: 241 - protein, 4 aa, synthetic construct
  REGION 1..4: note = an artificially synthesized sequence
GVPL

SEQ ID NO: 242 - protein, 4 aa, synthetic construct
  REGION 1..4: note = an artificially synthesized sequence
HVPL

SEQ ID NO: 243 - protein, 4 aa, synthetic construct
  REGION 1..4: note = an artificially synthesized sequence
IVPL

SEQ ID NO: 244 - protein, 4 aa, synthetic construct
  REGION 1..4: note = an artificially synthesized sequence
KVPL

SEQ ID NO: 245 - protein, 4 aa, synthetic construct
  REGION 1..4: note = an artificially synthesized sequence
LVPL

SEQ ID NO: 246 - protein, 4 aa, synthetic construct
  REGION 1..4: note = an artificially synthesized sequence
MVPL

SEQ ID NO: 247 - protein, 4 aa, synthetic construct
  REGION 1..4: note = an artificially synthesized sequence
NVPL

SEQ ID NO: 248 - protein, 4 aa, synthetic construct
  REGION 1..4: note = an artificially synthesized sequence
PVPL

SEQ ID NO: 249 - protein, 4 aa, synthetic construct
  REGION 1..4: note = an artificially synthesized sequence
QVPL

SEQ ID NO: 250 - protein, 4 aa, synthetic construct
  REGION 1..4: note = an artificially synthesized sequence
RVPL

SEQ ID NO: 251 - protein, 4 aa, synthetic construct
  REGION 1..4: note = an artificially synthesized sequence
SVPL

SEQ ID NO: 252 - protein, 4 aa, synthetic construct
  REGION 1..4: note = an artificially synthesized sequence
TVPL

SEQ ID NO: 253 - protein, 4 aa, synthetic construct
  REGION 1..4: note = an artificially synthesized sequence
VVPL

SEQ ID NO: 254 - protein, 4 aa, synthetic construct
  REGION 1..4: note = an artificially synthesized sequence
WVPL

SEQ ID NO: 255 - protein, 4 aa, synthetic construct
  REGION 1..4: note = an artificially synthesized sequence
YVPL

SEQ ID NO: 256 - protein, 4 aa, synthetic construct
  REGION 1..4: note = an artificially synthesized sequence
VAPL

SEQ ID NO: 257 - protein, 4 aa, synthetic construct
  REGION 1..4: note = an artificially synthesized sequence
VDPL

SEQ ID NO: 258 - protein, 4 aa, synthetic construct
  REGION 1..4: note = an artificially synthesized sequence
VEPL

SEQ ID NO: 259 - protein, 4 aa, synthetic construct
  REGION 1..4: note = an artificially synthesized sequence
VFPL

SEQ ID NO: 260 - protein, 4 aa, synthetic construct
  REGION 1..4: note = an artificially synthesized sequence
VGPL

SEQ ID NO: 261 - protein, 4 aa, synthetic construct
  REGION 1..4: note = an artificially synthesized sequence
VHPL

SEQ ID NO: 262 - protein, 4 aa, synthetic construct
  REGION 1..4: note = an artificially synthesized sequence
VIPL

SEQ ID NO: 263 - protein, 4 aa, synthetic construct
  REGION 1..4: note = an artificially synthesized sequence
VKPL

SEQ ID NO: 264 - protein, 4 aa, synthetic construct
  REGION 1..4: note = an artificially synthesized sequence
VLPL

SEQ ID NO: 265 - protein, 4 aa, synthetic construct
  REGION 1..4: note = an artificially synthesized sequence
VMPL

SEQ ID NO: 266 - protein, 4 aa, synthetic construct
  REGION 1..4: note = an artificially synthesized sequence
VNPL

SEQ ID NO: 267 - protein, 4 aa, synthetic construct
  REGION 1..4: note = an artificially synthesized sequence
VPPL

SEQ ID NO: 268 - protein, 4 aa, synthetic construct
  REGION 1..4: note = an artificially synthesized sequence
VQPL

SEQ ID NO: 269 - protein, 4 aa, synthetic construct
  REGION 1..4: note = an artificially synthesized sequence
VRPL

SEQ ID NO: 270 - protein, 4 aa, synthetic construct
  REGION 1..4: note = an artificially synthesized sequence
VSPL

SEQ ID NO: 271 - protein, 4 aa, synthetic construct
  REGION 1..4: note = an artificially synthesized sequence
VTPL

SEQ ID NO: 272 - protein, 4 aa, synthetic construct
  REGION 1..4: note = an artificially synthesized sequence
VVPL

SEQ ID NO: 273 - protein, 4 aa, synthetic construct
  REGION 1..4: note = an artificially synthesized sequence
VWPL

SEQ ID NO: 274 - protein, 4 aa, synthetic construct
  REGION 1..4: note = an artificially synthesized sequence
VYPL

SEQ ID NO: 275 - protein, 4 aa, synthetic construct
  REGION 1..4: note = an artificially synthesized sequence
FPMF

SEQ ID NO: 276 - protein, 4 aa, synthetic construct
  REGION 1..4: note = an artificially synthesized sequence
FAMF

SEQ ID NO: 277 - protein, 4 aa, synthetic construct
  REGION 1..4: note = an artificially synthesized sequence
FDMF

SEQ ID NO: 278 - protein, 4 aa, synthetic construct
  REGION 1..4: note = an artificially synthesized sequence
FEMF

SEQ ID NO: 279 - protein, 4 aa, synthetic construct
  REGION 1..4: note = an artificially synthesized sequence
FFMF

SEQ ID NO: 280 - protein, 4 aa, synthetic construct
  REGION 1..4: note = an artificially synthesized sequence
FGMF

SEQ ID NO: 281 - protein, 4 aa, synthetic construct
  REGION 1..4: note = an artificially synthesized sequence
FHMF

SEQ ID NO: 282 - protein, 4 aa, synthetic construct
  REGION 1..4: note = an artificially synthesized sequence
FIMF

SEQ ID NO: 283 - protein, 4 aa, synthetic construct
  REGION 1..4: note = an artificially synthesized sequence
FKMF

SEQ ID NO: 284 - protein, 4 aa, synthetic construct
  REGION 1..4: note = an artificially synthesized sequence
FLMF

SEQ ID NO: 285 - protein, 4 aa, synthetic construct
  REGION 1..4: note = an artificially synthesized sequence
FMMF

SEQ ID NO: 286 - protein, 4 aa, synthetic construct
  REGION 1..4: note = an artificially synthesized sequence
FNMF

SEQ ID NO: 287 - protein, 4 aa, synthetic construct
  REGION 1..4: note = an artificially synthesized sequence
FQMF

SEQ ID NO: 288 - protein, 4 aa, synthetic construct
  REGION 1..4: note = an artificially synthesized sequence
FRMF

SEQ ID NO: 289 - protein, 4 aa, synthetic construct
  REGION 1..4: note = an artificially synthesized sequence
FSMF

SEQ ID NO: 290 - protein, 4 aa, synthetic construct
  REGION 1..4: note = an artificially synthesized sequence
FTMF

SEQ ID NO: 291 - protein, 4 aa, synthetic construct
  REGION 1..4: note = an artificially synthesized sequence
FVMF

SEQ ID NO: 292 - protein, 4 aa, synthetic construct
  REGION 1..4: note = an artificially synthesized sequence
FWMF

SEQ ID NO: 293 - protein, 4 aa, synthetic construct
  REGION 1..4: note = an artificially synthesized sequence
FYMF

SEQ ID NO: 294 - protein, 4 aa, synthetic construct
  REGION 1..4: note = an artificially synthesized sequence
AFPM

SEQ ID NO: 295 - protein, 4 aa, synthetic construct
  REGION 1..4: note = an artificially synthesized sequence
DFPM

SEQ ID NO: 296 - protein, 4 aa, synthetic construct
  REGION 1..4: note = an artificially synthesized sequence
EFPM

SEQ ID NO: 297 - protein, 4 aa, synthetic construct
  REGION 1..4: note = an artificially synthesized sequence
FFPM

SEQ ID NO: 298 - protein, 4 aa, synthetic construct
  REGION 1..4: note = an artificially synthesized sequence
GFPM

SEQ ID NO: 299 - protein, 4 aa, synthetic construct
  REGION 1..4: note = an artificially synthesized sequence
HFPM

SEQ ID NO: 300 - protein, 4 aa, synthetic construct
  REGION 1..4: note = an artificially synthesized sequence
IFPM

SEQ ID NO: 301 - protein, 4 aa, synthetic construct
  REGION 1..4: note = an artificially synthesized sequence
KFPM

SEQ ID NO: 302 - protein, 4 aa, synthetic construct
  REGION 1..4: note = an artificially synthesized sequence
LFPM

SEQ ID NO: 303 - protein, 4 aa, synthetic construct
  REGION 1..4: note = an artificially synthesized sequence
MFPM

SEQ ID NO: 304 - protein, 4 aa, synthetic construct
  REGION 1..4: note = an artificially synthesized sequence
NFPM

SEQ ID NO: 305 - protein, 4 aa, synthetic construct
  REGION 1..4: note = an artificially synthesized sequence
PFPM

SEQ ID NO: 306 - protein, 4 aa, synthetic construct
  REGION 1..4: note = an artificially synthesized sequence
QFPM

SEQ ID NO: 307 - protein, 4 aa, synthetic construct
  REGION 1..4: note = an artificially synthesized sequence
RFPM

SEQ ID NO: 308 - protein, 4 aa, synthetic construct
  REGION 1..4: note = an artificially synthesized sequence
SFPM

SEQ ID NO: 309 - protein, 4 aa, synthetic construct
  REGION 1..4: note = an artificially synthesized sequence
TFPM

SEQ ID NO: 310 - protein, 4 aa, synthetic construct
  REGION 1..4: note = an artificially synthesized sequence
VFPM

SEQ ID NO: 311 - protein, 4 aa, synthetic construct
  REGION 1..4: note = an artificially synthesized sequence
WFPM

SEQ ID NO: 312 - protein, 4 aa, synthetic construct
  REGION 1..4: note = an artificially synthesized sequence
YFPM

SEQ ID NO: 313 - protein, 4 aa, synthetic construct
  REGION 1..4: note = an artificially synthesized sequence
FAPM

SEQ ID NO: 314 - protein, 4 aa, synthetic construct
  REGION 1..4: note = an artificially synthesized sequence
FDPM

SEQ ID NO: 315 - protein, 4 aa, synthetic construct
  REGION 1..4: note = an artificially synthesized sequence
FEPM

SEQ ID NO: 316 - protein, 4 aa, synthetic construct
  REGION 1..4: note = an artificially synthesized sequence
FFPM

SEQ ID NO: 317 - protein, 4 aa, synthetic construct
  REGION 1..4: note = an artificially synthesized sequence
FGPM

SEQ ID NO: 318 - protein, 4 aa, synthetic construct
  REGION 1..4: note = an artificially synthesized sequence
FHPM

SEQ ID NO: 319 - protein, 4 aa, synthetic construct
  REGION 1..4: note = an artificially synthesized sequence
FIPM

SEQ ID NO: 320 - protein, 4 aa, synthetic construct
  REGION 1..4: note = an artificially synthesized sequence
FKPM

SEQ ID NO: 321 - protein, 4 aa, synthetic construct
  REGION 1..4: note = an artificially synthesized sequence
FLPM

SEQ ID NO: 322 - protein, 4 aa, synthetic construct
  REGION 1..4: note = an artificially synthesized sequence
FMPM

SEQ ID NO: 323 - protein, 4 aa, synthetic construct
  REGION 1..4: note = an artificially synthesized sequence
FNPM

SEQ ID NO: 324 - protein, 4 aa, synthetic construct
  REGION 1..4: note = an artificially synthesized sequence
FPPM

SEQ ID NO: 325 - protein, 4 aa, synthetic construct
  REGION 1..4: note = an artificially synthesized sequence
FQPM

SEQ ID NO: 326 - protein, 4 aa, synthetic construct
  REGION 1..4: note = an artificially synthesized sequence
FRPM

SEQ ID NO: 327 - protein, 4 aa, synthetic construct
  REGION 1..4: note = an artificially synthesized sequence
FSPM

SEQ ID NO: 328 - protein, 4 aa, synthetic construct
  REGION 1..4: note = an artificially synthesized sequence
FTPM

SEQ ID NO: 329 - protein, 4 aa, synthetic construct
  REGION 1..4: note = an artificially synthesized sequence
FVPM

SEQ ID NO: 330 - protein, 4 aa, synthetic construct
  REGION 1..4: note = an artificially synthesized sequence
FWPM

SEQ ID NO: 331 - protein, 4 aa, synthetic construct
  REGION 1..4: note = an artificially synthesized sequence
FYPM

SEQ ID NO: 332 - protein, 4 aa, synthetic construct
  REGION 1..4: note = an artificially synthesized sequence
FPMF

SEQ ID NO: 333 - protein, 4 aa, synthetic construct
  REGION 1..4: note = an artificially synthesized sequence
FAMF

SEQ ID NO: 334 - protein, 4 aa, synthetic construct
  REGION 1..4: note = an artificially synthesized sequence
FDMF

SEQ ID NO: 335 - protein, 4 aa, synthetic construct
  REGION 1..4: note = an artificially synthesized sequence
FEMF

SEQ ID NO: 336 - protein, 4 aa, synthetic construct
  REGION 1..4: note = an artificially synthesized sequence
FFMF

SEQ ID NO: 337 - protein, 4 aa, synthetic construct
  REGION 1..4: note = an artificially synthesized sequence
FGMF

SEQ ID NO: 338 - protein, 4 aa, synthetic construct
  REGION 1..4: note = an artificially synthesized sequence
FHMF

SEQ ID NO: 339 - protein, 4 aa, synthetic construct
  REGION 1..4: note = an artificially synthesized sequence
FIMF

SEQ ID NO: 340 - protein, 4 aa, synthetic construct
  REGION 1..4: note = an artificially synthesized sequence
FKMF

SEQ ID NO: 341 - protein, 4 aa, synthetic construct
  REGION 1..4: note = an artificially synthesized sequence
FLMF

SEQ ID NO: 342 - protein, 4 aa, synthetic construct
  REGION 1..4: note = an artificially synthesized sequence
FMMF

SEQ ID NO: 343 - protein, 4 aa, synthetic construct
  REGION 1..4: note = an artificially synthesized sequence
FNMF

SEQ ID NO: 344 - protein, 4 aa, synthetic construct
  REGION 1..4: note = an artificially synthesized sequence
FQMF

SEQ ID NO: 345 - protein, 4 aa, synthetic construct
  REGION 1..4: note = an artificially synthesized sequence
FRMF

SEQ ID NO: 346 - protein, 4 aa, synthetic construct
  REGION 1..4: note = an artificially synthesized sequence
FSMF

SEQ ID NO: 347 - protein, 4 aa, synthetic construct
  REGION 1..4: note = an artificially synthesized sequence
FTMF

SEQ ID NO: 348 - protein, 4 aa, synthetic construct
  REGION 1..4: note = an artificially synthesized sequence
FVMF

SEQ ID NO: 349 - protein, 4 aa, synthetic construct
  REGION 1..4: note = an artificially synthesized sequence
FWMF

SEQ ID NO: 350 - protein, 4 aa, synthetic construct
  REGION 1..4: note = an artificially synthesized sequence
FYMF

SEQ ID NO: 351 - protein, 4 aa, synthetic construct
  REGION 1..4: note = an artificially synthesized sequence
AFPM

SEQ ID NO: 352 - protein, 4 aa, synthetic construct
  REGION 1..4: note = an artificially synthesized sequence
DFPM

SEQ ID NO: 353 - protein, 4 aa, synthetic construct
  REGION 1..4: note = an artificially synthesized sequence
EFPM

SEQ ID NO: 354 - protein, 4 aa, synthetic construct
  REGION 1..4: note = an artificially synthesized sequence
FFPM

SEQ ID NO: 355 - protein, 4 aa, synthetic construct
  REGION 1..4: note = an artificially synthesized sequence
GFPM

SEQ ID NO: 356 - protein, 4 aa, synthetic construct
  REGION 1..4: note = an artificially synthesized sequence
HFPM

SEQ ID NO: 357 - protein, 4 aa, synthetic construct
  REGION 1..4: note = an artificially synthesized sequence
IFPM

SEQ ID NO: 358 - protein, 4 aa, synthetic construct
  REGION 1..4: note = an artificially synthesized sequence
KFPM

SEQ ID NO: 359 - protein, 4 aa, synthetic construct
  REGION 1..4: note = an artificially synthesized sequence
LFPM

SEQ ID NO: 360 - protein, 4 aa, synthetic construct
  REGION 1..4: note = an artificially synthesized sequence
MFPM

SEQ ID NO: 361 - protein, 4 aa, synthetic construct
  REGION 1..4: note = an artificially synthesized sequence
NFPM

SEQ ID NO: 362 - protein, 4 aa, synthetic construct
  REGION 1..4: note = an artificially synthesized sequence
PFPM

SEQ ID NO: 363 - protein, 4 aa, synthetic construct
  REGION 1..4: note = an artificially synthesized sequence
QFPM

SEQ ID NO: 364 - protein, 4 aa, synthetic construct
  REGION 1..4: note = an artificially synthesized sequence
RFPM

SEQ ID NO: 365 - protein, 4 aa, synthetic construct
  REGION 1..4: note = an artificially synthesized sequence
SFPM

SEQ ID NO: 366 - protein, 4 aa, synthetic construct
  REGION 1..4: note = an artificially synthesized sequence
TFPM

SEQ ID NO: 367 - protein, 4 aa, synthetic construct
  REGION 1..4: note = an artificially synthesized sequence
VFPM

SEQ ID NO: 368 - protein, 4 aa, synthetic construct
  REGION 1..4: note = an artificially synthesized sequence
WFPM

SEQ ID NO: 369 - protein, 4 aa, synthetic construct
  REGION 1..4: note = an artificially synthesized sequence
YFPM

SEQ ID NO: 370 - protein, 4 aa, synthetic construct
  REGION 1..4: note = an artificially synthesized sequence
FAPM

SEQ ID NO: 371 - protein, 4 aa, synthetic construct
  REGION 1..4: note = an artificially synthesized sequence
FDPM

SEQ ID NO: 372 - protein, 4 aa, synthetic construct
  REGION 1..4: note = an artificially synthesized sequence
FEPM

SEQ ID NO: 373 - protein, 4 aa, synthetic construct
  REGION 1..4: note = an artificially synthesized sequence
FFPM

SEQ ID NO: 374 - protein, 4 aa, synthetic construct
  REGION 1..4: note = an artificially synthesized sequence
FGPM

SEQ ID NO: 375 - protein, 4 aa, synthetic construct
  REGION 1..4: note = an artificially synthesized sequence
FHPM

SEQ ID NO: 376 - protein, 4 aa, synthetic construct
  REGION 1..4: note = an artificially synthesized sequence
FIPM

SEQ ID NO: 377 - protein, 4 aa, synthetic construct
  REGION 1..4: note = an artificially synthesized sequence
FKPM

SEQ ID NO: 378 - protein, 4 aa, synthetic construct
  REGION 1..4: note = an artificially synthesized sequence
FLPM

SEQ ID NO: 379 - protein, 4 aa, synthetic construct
  REGION 1..4: note = an artificially synthesized sequence
FMPM

SEQ ID NO: 380 - protein, 4 aa, synthetic construct
  REGION 1..4: note = an artificially synthesized sequence
FNPM

SEQ ID NO: 381 - protein, 4 aa, synthetic construct
  REGION 1..4: note = an artificially synthesized sequence
FPPM

SEQ ID NO: 382 - protein, 4 aa, synthetic construct
  REGION 1..4: note = an artificially synthesized sequence
FQPM

SEQ ID NO: 383 - protein, 4 aa, synthetic construct
  REGION 1..4: note = an artificially synthesized sequence
FRPM

SEQ ID NO: 384 - protein, 4 aa, synthetic construct
  REGION 1..4: note = an artificially synthesized sequence
FSPM

SEQ ID NO: 385 - protein, 4 aa, synthetic construct
  REGION 1..4: note = an artificially synthesized sequence
FTPM

SEQ ID NO: 386 - protein, 4 aa, synthetic construct
  REGION 1..4: note = an artificially synthesized sequence
FVPM

SEQ ID NO: 387 - protein, 4 aa, synthetic construct
  REGION 1..4: note = an artificially synthesized sequence
FWPM

SEQ ID NO: 388 - protein, 4 aa, synthetic construct
  REGION 1..4: note = an artificially synthesized sequence
FYPM